(19) United States
(12) Patent Application Publication
(10) Pub. No.: US 2023/0357789 A1
(43) Pub. Date: Nov. 9, 2023

(54) METHODS AND COMPOSITIONS FOR IMPROVING RESISTANCE TO FUSARIUM HEAD BLIGHT

(71) Applicants: Pairwise Plants Services, Inc., Durham, NC (US); Monsanto Technology LLC, St. Louis, MO (US)

(72) Inventors: Marisa Miller, Durham, NC (US); John Pitkin, St. Louis, MO (US)

(21) Appl. No.: 18/296,392

(22) Filed: Apr. 6, 2023

Related U.S. Application Data
(60) Provisional application No. 63/328,313, filed on Apr. 7, 2022.

(51) Int. Cl. C12N 15/82

(52) U.S. Cl. CPC C12N 15/8282

(57) ABSTRACT

This invention relates to compositions and methods for modifying Ribosomal Protein L3 (RPL3) genes in plants, optionally to improve resistance to Fusarium head blight. The invention further relates to plants having improved resistance to Fusarium head blight produced using the methods and compositions of the invention.

### STATEMENT REGARDING ELECTRONIC FILING OF A SEQUENCE LISTING

A Sequence Listing in XML format, entitled 1499-35_ST26.xml, 278,127 bytes in size, generated on Mar. 24, 2023 and filed herewith, is hereby incorporated by reference into the specification for its disclosures.

### STATEMENT OF PRIORITY

This application claims the benefit, under 35 U.S.C. § 119 (e), of U.S. Provisional Application No. 63/328,313 filed on Apr. 7, 2022, the entire contents of which is incorporated by reference herein.

### FIELD OF THE INVENTION

This invention relates to compositions and methods for modifying Ribosomal Protein L3 (RPL3) genes in plants, optionally to improve resistance to Fusarium head blight. The invention further relates to plants having improved resistance to Fusarium head blight produced using the methods and compositions of the invention.

### BACKGROUND OF THE INVENTION

A combination of fungicides, cultural practices, and genetic resistance are used to control Fusarium head blight (FHB). Resistance to FHB is quantitative and the Fhb1 QTL is the most commonly used source of resistance. Fungicides are generally subject to the development of resistance by pathogens, and therefore, grow less effective or ineffective over time. Current resistance loci (such as the Fhb1 QTL) only provide partial, quantitative resistance. New sources of resistance are desired that optionally, could be stacked with existing loci to enhance resistance to FHB.

The present invention addresses the shortcomings in the art by providing new compositions and methods for improving FHB resistance in plants.

### SUMMARY OF THE INVENTION

One aspect of the invention provides a plant or part thereof comprising at least one mutation in an endogenous Ribosomal Protein L3 (RPL3) gene that encodes a RPL3 polypeptide, optionally wherein the at least one mutation results in no detectable RPL3 polypeptide or a RPL3 polypeptide having reduced binding to a trichothecene mycotoxin, optionally wherein the at least one mutation is a non-natural mutation.

A second aspect of the invention provides a plant cell, comprising an editing system the editing system comprising: (a) a CRISPR-Cas effector protein; and (b) a guide nucleic acid comprising a spacer sequence with complementarity to an endogenous target gene encoding a Ribosomal Protein L3 (RPL3) polypeptide.

A third aspect of the invention provides a plant cell comprising at least one mutation in one or more endogenous Ribosomal Protein+ L3 (RPL3) genes, wherein the at least one mutation is a substitution, insertion and/or a deletion that is introduced using an editing system that comprises a nucleic acid binding domain that binds to a target site in the one or more endogenous RPL3 genes, optionally wherein the at least one mutation is a non-natural mutation.

A fourth aspect of the invention provides a method of providing a plurality of plants (i) comprising a mutated RPL3 polypeptide having reduced binding to a trichothecene mycotoxin, (ii) having increased resistance to fusarium head blight (FHB) (e.g., increased resistance to the FHB causal agent, Fusarium graminearum), (iii) having a reduced Fusarium graminearum load, and/or (iv) having increased tolerance to deoxynivalenol (DON), the method comprising planting two or more plants of the invention in a growing area, thereby providing a plurality of plants (i) comprising a mutated RPL3 polypeptide having reduced binding to a trichothecene mycotoxin, (ii) having increased resistance to fusarium head blight (FHB) (e.g., increased resistance to the FHB causal agent, Fusarium graminearum), (iii) having a reduced Fusarium graminearum load, and/or (iv) having increased tolerance to deoxynivalenol (DON) as compared to a plurality of control plants devoid of the at least one mutation in the one or more endogenous RPL3 genes as compared to a plurality of control plants not comprising the at least one mutation.

A fifth aspect of the invention provides a method of producing/breeding a transgene-free edited plant, comprising: crossing the plant of the invention with a transgene free plant, thereby introducing the at least one mutation into the plant that is transgene-free; and selecting a progeny plant that comprises the at least one mutation and is transgene-free, thereby producing a transgene free edited plant, optionally wherein the at least one mutation is a non-natural mutation.

A sixth aspect provides a method of generating variation in a Ribosomal Protein L3 (RPL3) gene, comprising: introducing an editing system into a plant cell, wherein the editing system is targeted to a region of a RPL3 gene that encodes a RPL3 polypeptide, and contacting the region of the RPL3 gene with the editing system, thereby introducing a mutation into the RPL3 gene and generating variation in the RPL3 gene of the plant cell.

A seventh aspect of the invention provides a method for editing a specific site in the genome of a plant cell, the method comprising: cleaving, in a site-specific manner, a target site within an endogenous Ribosomal Protein L3 (RPL3) gene in the plant cell, the endogenous RPL3 gene: (a) comprising a nucleotide sequence having at least 80% sequence identity to any one of SEQ ID NO:72, 73, 75, 76, 78, 79, 80, 81, 83, 84, 86, or 87, (b) comprising a region having at least 80% sequence identity to any one of the nucleotide sequences of SEQ ID NOs:89-139, and/or (c) encoding an amino acid sequence having at least 80% sequence identity to SEQ ID NO:74, 77, 82, 85, or 88, thereby generating an edit in the endogenous RPL3 gene of the plant cell and producing a plant cell comprising the edit in the endogenous RPL3 gene.

An eighth aspect provides a method for making a plant, the method comprising: (a) contacting a population of plant cells comprising an endogenous Ribosomal Protein L3 (RPL3) gene with a nuclease linked to a nucleic acid binding domain (e.g., editing system) that binds to a sequence (i) having at least 80% sequence identity to any one of the nucleotide sequences of SEQ ID NO:72, 73, 75, 76, 78, 79, 80, 81, 83, 84, 86, or 87, (ii) comprising a region having at least 80% sequence identity to a nucleotide sequence of any one of SEQ ID NOs:89-139, and/or (iii) encoding an amino acid sequence having at least 80% sequence identity to SEQ ID NO:74, 77, 82, 85, or 88; (b) selecting a plant cell from the population of plant cells in which an endogenous RPL3 gene has been mutated, thereby producing a plant cell comprising a mutation in the endogenous RPL3 gene; and (c) growing the selected plant cell into a plant comprising the mutation in the endogenous gene encoding a RPL3 polypeptide, wherein the mutation reduces or eliminates the ability of the RPL3 polypeptide to bind a trichothecene mycotoxin, optionally deoxynivalenol (DON).

A ninth aspect provides a method for increasing resistance to fusarium head blight (FHB) (e.g., increased resistance to the FHB causal agent, Fusarium graminearum) and/or tolerance to a trichothecene mycotoxin, optionally deoxynivalenol (DON), in a plant, comprising: (a) contacting a plant cell comprising a endogenous Ribosomal Protein L3 (RPL3) gene with a nuclease targeted to the endogenous RPL3 gene, wherein the nuclease is linked to a nucleic acid binding domain (e.g., editing system) that binds to a target site in the endogenous RPL3 gene and the endogenous RPL3 gene: (i) comprises a nucleotide sequence having at least 80% sequence identity to any one of SEQ ID NO:72, 73, 75, 76, 78, 79, 80, 81, 83, 84, 86, or 87, (ii) comprises a region having at least 80% sequence identity to any one of the nucleotide sequences of SEQ ID NOs:89-139, and/or (iii) encodes an amino acid sequence having at least 80% sequence identity to SEQ ID NO:74, 77, 82, 85, or 88, thereby producing a plant cell comprising a mutation in the endogenous RPL3 gene; and (b) growing the plant cell into a plant comprising the mutation in the endogenous RPL3 gene, thereby increasing resistance to FHB and/or increasing tolerance to the trichothecene mycotoxin in the plant.

A tenth aspect provides a method of producing a plant or part thereof comprising at least one cell having a mutated Ribosomal Protein L3 (RPL3) gene, the method comprising contacting a target site in an endogenous RPL3 gene in the plant or plant part with a nuclease comprising a cleavage domain and a nucleic acid binding domain, wherein the nucleic acid binding domain binds to a target site in the endogenous RPL3 gene, wherein the endogenous RPL3 gene (a) comprises a sequence having at least 80% sequence identity to any one of the nucleotide sequences of SEQ ID NO:72, 73, 75, 76, 78, 79, 80, 81, 83, 84, 86, or 87; (b) comprises a region having at least 80% identity to any one of SEQ ID NOs:89-139; and/or (c) encodes an amino acid sequence having at least 80% sequence identity to SEQ ID NO:74, 77, 82, 85, or 88, thereby producing the plant or part thereof comprising at least one cell having a mutation in the endogenous RPL3 gene.

An eleventh aspect of the invention provides a method for producing a plant or part thereof comprising a mutation in an endogenous RPL3 gene that produces a mutated RPL3 polypeptide having reduced binding to a trichothecene mycotoxin, the method comprising contacting a target site in the endogenous RPL3 gene in the plant or plant part with a nuclease comprising a cleavage domain and a nucleic acid binding domain, wherein the nucleic acid binding domain of the nuclease binds to a target site in the endogenous RPL3 gene, wherein the endogenous RPL3 gene: (a) comprises a sequence having at least 80% sequence identity to any one of the nucleotide sequences of SEQ ID NO:72, 73, 75, 76, 78, 79, 80, 81, 83, 84, 86, or 87; (b) comprises a region having at least 80% identity to any one of SEQ ID NOs:89-139; and/or (c) encodes an amino acid sequence having at least 80% sequence identity to SEQ ID NO:74, 77, 82, 85, or 88, thereby producing a plant or part thereof having a mutation in the endogenous gene that produces a mutated RPL3 polypeptide having reduced binding to the trichothecene mycotoxin.

A twelfth aspect provides a guide nucleic acid that binds to a target site in an endogenous Ribosomal Protein L3 (RPL3) gene having a gene identification number (gene ID) of TraesCS4A02G208000 (SEQ ID NO:72), TraesCS4B02G111500 (SEQ ID NO:75), TraesCS4D02G109200 (SEQ ID NO:78), TraesCS5A02G112400 (SEQ ID NO:80), TraesCS5B02G118400 (SEQ ID NO:83), and/or TraesCS5D02G129200 (SEQ ID NO:86), optionally wherein the target site is in a region of the endogenous RPL3 gene having at least 80% sequence identity to any one of SEQ ID NOs:89-139.

In a thirteenth aspect, a system is provided that comprises a guide nucleic acid of the invention and a CRISPR-Cas effector protein that associates with the guide nucleic acid.

A fourteenth aspect provides a gene editing system comprising a CRISPR-Cas effector protein in association with a guide nucleic acid, wherein the guide nucleic acid comprises a spacer sequence that binds to an endogenous Ribosomal Protein L3 (RPL3) gene.

In a fifteenth aspect, a complex comprising a guide nucleic acid and a CRISPR-Cas effector protein comprising a cleavage domain is provided, wherein the guide nucleic acid binds to a target site in a RPL3 gene, wherein the endogenous RPL3 gene (a) comprises a sequence having at least 80% sequence identity to any one of the nucleotide sequences of SEQ ID NO:72, 73, 75, 76, 78, 79, 80, 81, 83, 84, 86, or 87; (b) comprises a region having at least 80% identity to any one of SEQ ID NOs:89-139; and/or (c) encodes an amino acid sequence having at least 80% sequence identity to SEQ ID NO:74, 77, 82, 85, or 88, and the cleavage domain cleaves a target strand in the RPL3 gene.

In a sixteenth aspect, an expression cassette is provided, the expression cassette comprising (a) a polynucleotide encoding CRISPR-Cas effector protein comprising a cleavage domain and (b) a guide nucleic acid that binds to a target site in a RPL3 gene, wherein the guide nucleic acid comprises a spacer sequence that is complementary to and binds to (i) a portion of a nucleic acid having at least 80% sequence identity to any one of the nucleotide sequences of SEQ ID NO:72, 73, 75, 76, 78, 79, 80, 81, 83, 84, 86, or 87; (ii) a portion of a nucleic acid having at least 80% sequence identity to any one of the nucleotide sequences of SEQ ID NOs:89-139; and/or (iii) a portion of a nucleic acid encoding an amino acid sequence having at least 80% sequence identity to SEQ ID NO:74, 77, 82, 85, or 88.

In a seventeenth aspect, a method of creating a mutation in an endogenous Ribosomal Protein L3 (RPL3) gene in a plant is provided, the method comprising (a) targeting a gene editing system to a portion of the one or more endogenous RPL3 genes that comprises a sequence having at least 90% sequence identity to any one of SEQ ID NOs:89-139; and (b) selecting a plant that comprises a modification located in a region of the one or more endogenous RPL3 genes having at least 90% sequence identity to any one of SEQ ID NOs:89-139.

In an eighteenth aspect, plants are provided that comprise in their genome one or more mutated Ribosomal Protein L3 (RPL3) genes produced by the methods of the invention.

A further aspect of the invention provides a plant or plant part thereof comprising at least one mutation in at least one endogenous Ribosomal Protein L3 (RPL3) gene having a gene identification number (gene ID) of TraesCS4A02G208000 (SEQ ID NO:72), TraesCS4B02G111500 (SEQ ID NO:75), TraesCS4D02G109200 (SEQ ID NO:78), TraesCS5A02G112400 (SEQ ID NO:80), TraesCS5B02G118400 (SEQ ID NO:83), and/or TraesCS5D02G129200 (SEQ ID NO:86), optionally wherein the at least one mutation is a non-natural mutation.

A nineteenth aspect of the invention provides a nucleic acid comprising a mutated Ribosomal Protein L3 (RPL3) gene, optionally wherein the mutated RPL3 gene comprises a mutation in Exon 3.

A twentieth aspect provides a mutated nucleic acid encoding a Ribosomal Protein L3 (RPL3) polypeptide, optionally the mutation is in Exon 3 of the RPL3 gene, wherein the mutation results in no detectable RPL3 polypeptide or a RPL3 polypeptide having reduced binding to a trichothecene mycotoxin.

In an additional aspect, a mutated RPL3 gene is provided, wherein the mutated endogenous RPL3 gene comprises a nucleic acid sequence having at least 90% sequence identity to any one of SEQ ID NOs:147, 149, 151, 153, or 155 and/or encodes a mutated RPL3 polypeptide having at least 90% sequence identity to any one of SEQ ID NOs:148, 150, 152, 154, or 156, optionally wherein the mutated endogenous RPL3 gene comprises a non-natural mutation.

Further provided are plants comprising in their genome one or more Ribosomal Protein L3 (RPL3) genes having a mutation produced by the methods of the invention, optionally wherein the plant (a) exhibits increased resistance to fusarium head blight (FHB) (e.g., increased resistance to the FHB causal agent, Fusarium graminearum), (b) exhibits a decreased amount of Fusarium graminearum, e.g., a decreased fusarium load; (c) exhibits increased tolerance to deoxynivalenol (DON); and/or (d) produces seed that accumulates less trichothecene mycotoxin (e.g., DON) upon infection with FHB (infection by the causal agent of FHB (Fusarium graminearum), optionally wherein the mutation may be a non-natural mutation.

Additionally provided are polypeptides, polynucleotides, nucleic acid constructs, expression cassettes and vectors for making a plant of this invention.

These and other aspects of the invention are set forth in more detail in the description of the invention below.

### BRIEF DESCRIPTION OF THE SEQUENCES

SEQ ID NOs:1-17 are exemplary Cas12a amino acid sequences useful with this invention.

SEQ ID NOs:18-20 are exemplary Cas12a nucleotide sequences useful with this invention.

SEQ ID NO:21-22 are exemplary regulatory sequences encoding a promoter and intron.

SEQ ID NOs:23-29 are exemplary cytosine deaminase sequences useful with this invention.

SEQ ID NOs:30-40 are exemplary adenine deaminase amino acid sequences useful with this invention.

SEQ ID NO:41 is an exemplary uracil-DNA glycosylase inhibitor (UGI) sequences useful with this invention.

SEQ ID NOs:42-44 provides an example of a protospacer adjacent motif position for a Type V CRISPR-Cas12a nuclease.

SEQ ID NOs:45-47 provide example peptide tags and affinity polypeptides useful with this invention.

SEQ ID NOs:48-58 provide example RNA recruiting motifs and corresponding affinity polypeptides useful with this invention.

SEQ ID NOs:59-60 are exemplary Cas9 polypeptide sequences useful with this invention.

SEQ ID NOs:61-71 are exemplary Cas9 polynucleotide sequences useful with this invention.

SEQ ID NO:72, 75, 78, 80, 83, or 86 are example RPL3 genomic sequences from wheat.

SEQ ID NO:73, 76, 79, 81, 84 or 87 are example RPL3 cDNA sequences from wheat.

SEQ ID NO:74, 77, 82, 85 or 88 are example RPL3 polypeptide sequences from wheat.

SEQ ID NOs:89-139 are example portions or regions of a RPL3 genomic and coding sequences.

SEQ ID NOs:140-146 are example spacer sequences for nucleic acid guides useful with this invention.

SEQ ID NOs:147, 149, 151, 153, and 155 are example mutated RPL3 genes.

SEQ ID NOs:148, 150, 152, 154, and 156 are example mutated RPL3 polypeptides encoded by SEQ ID NOs:147, 149, 151, 153, and 155, respectively.

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ± 10%, ± 5%, ± 1%, ± 0.5%, or even ± 0.1% of the specified value as well as the specified value. For example, “about X” where X is the measurable value, is meant to include X as well as variations of ± 10%, ± 5%, ± 1%, ± 0.5%, or even ± 0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

Recitation of ranges of values herein are merely intended to serve as a shorthand method of referring individually to each separate value falling within the range, unless otherwise indicated herein, and each separate value is incorporated into the specification as if it were individually recited herein. For example, if the range 10 to 15 is disclosed, then 11, 12, 13, and 14 are also disclosed.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “increased,” “enhance,” “enhanced,” “enhancing,” and “enhancement” (and grammatical variations thereof) describe an elevation of at least about 5%, 10%, 15%, 20%, 25%, 50%, 75%, 100%, 150%, 200%, 300%, 400%, 500% or more as compared to a control. For example, a plant comprising a mutation in a RPL3 gene as described herein can exhibit increased resistance to fusarium head blight (FHB) (e.g., increased resistance to the FHB causal agent, Fusarium graminearum, e.g., decreased symptoms of infection by the causal agent of FHB, Fusarium graminearum) as compared to a control plant devoid of the at least one mutation. A control plant is typically the same plant as the edited plant, but the control plant has not been similarly edited and therefore is devoid of the mutation. A control plant maybe an isogenic plant and/or a wild type plant. Thus, a control plant can be the same breeding line, variety, or cultivar as the subject plant into which a mutation as described herein is introgressed, but the control breeding line, variety, or cultivar is free of the mutation. In some embodiments, a comparison between a plant of the invention and a control plant is made under the same growth conditions, e.g., the same environmental conditions (soil, hydration, light, heat, nutrients, and the like).

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 35%, 50%, 75%, 80%, 85%, 90%, 95%, 96%, 97%, 98%, 99%, 99.5%, 99.6%, 99.7%, 99.8%, 99.9%, or 100% as compared to a control. In particular embodiments, the reduction can result in no or essentially no (i.e., an insignificant amount, e.g., less than about 10% or even 5%) detectable activity or amount. For example, a plant comprising a mutation in a RPL3 gene as described herein can exhibit reduced binding of a trichothecene mycotoxin to the mutated RPL3 polypeptide or exhibits a decreased amount of Fusarium graminearum, e.g., a decreased F. graminearum load as compared to a control plant devoid of the mutation.

As used herein, the terms “express,” “expresses,” “expressed” or “expression,” and the like, with respect to a nucleic acid molecule and/or a nucleotide sequence (e.g., RNA or DNA) indicates that the nucleic acid molecule and/or a nucleotide sequence is transcribed and, optionally, translated. Thus, a nucleic acid molecule and/or a nucleotide sequence may express a polypeptide of interest or, for example, a functional untranslated RNA.

A “heterologous” or a “recombinant” nucleotide sequence is a nucleotide sequence not naturally associated with a host cell into which it is introduced, including non- naturally occurring multiple copies of a naturally occurring nucleotide sequence. A “heterologous” nucleotide/polypeptide may originate from a foreign species, or, if from the same species, is substantially modified from its native form in composition and/or genomic locus by deliberate human intervention.

A “native” or “wild type” nucleic acid, nucleotide sequence, polypeptide or amino acid sequence refers to a naturally occurring or endogenous nucleic acid, nucleotide sequence, polypeptide or amino acid sequence. In some contexts, a “wild type” nucleic acid is a nucleic acid that is not edited as described herein and can differ from an “endogenous” gene that may be edited as described herein (e.g., a mutated endogenous gene). In some contexts, a “wild type” nucleic acid (e.g., unedited) may be heterologous to the organism in which the wild type nucleic acid is found (e.g., a transgenic organism). As an example, a “wild type endogenous Ribosomal Protein L3 (RPL3) gene” is a RPL3 gene that is naturally occurring in or endogenous to the reference organism, e.g., a plant, e.g., a wheat plant, a barley plant, oat plant, a spelt plant, and may be subject to modification as described herein, after which, such a modified endogenous gene is no longer wild type. In some embodiments, an endogenous RPL3 gene can be, but is not limited to, an endogenous RPL3A gene or an endogenous RPL3B gene, optionally wherein the endogenous RPL3A gene is a RPL3A-1 gene, a RPL3A-2 gene, and/or a RPL3A-3 gene, and/or the RPL3B gene is a RPL3B-1 gene, a RPL3B-2 gene, and/or a RPL3B-3 gene, optionally wherein the modification may be in one or more than one of a RPL3A gene, a RPL3A-1 gene, a RPL3A-2 gene, a RPL3A-3 gene, a RPL3B gene, a RPL3B-1 gene, a RPL3B-2 gene, and/or a RPL3B-3 gene.

As used herein, the term “heterozygous” refers to a genetic status wherein different alleles reside at corresponding loci on homologous chromosomes.

As used herein, the term “homozygous” refers to a genetic status wherein identical alleles reside at corresponding loci on homologous chromosomes.

As used herein, the term “allele” refers to one of two or more different nucleotides or nucleotide sequences that occur at a specific locus.

A “null allele” is a nonfunctional allele caused by a genetic mutation that results in a complete lack of production of the corresponding protein or produces a protein that is non-functional.

A “recessive mutation” is a mutation in a gene that produces a phenotype when homozygous but the phenotype is not observable when the locus is heterozygous.

A “dominant mutation” is a mutation in a gene that produces a mutant phenotype in the presence of a non-mutated copy of the gene. A dominant mutation may be a loss or a gain of function mutation, a hypomorphic mutation, a hypermorphic mutation or a weak loss of function or a weak gain of function.

A “dominant negative mutation” is a mutation that produces an altered gene product (e.g., having an aberrant function relative to wild type), which gene product adversely affects the function of the wild-type allele or gene product. For example, a “dominant negative mutation” may block a function of the wild type gene product. A dominant negative mutation may also be referred to as an “antimorphic mutation.”

A “semi-dominant mutation” refers to a mutation in which the penetrance of the phenotype in a heterozygous organism is less than that observed for a homozygous organism.

A “weak loss-of-function mutation” is a mutation that results in a gene product having partial function or reduced function (partially inactivated) as compared to the wildtype gene product.

A “hypomorphic mutation” is a mutation that results in a partial loss of gene function, which may occur through reduced expression (e.g., reduced protein and/or reduced RNA) or reduced functional performance (e.g., reduced activity), but not a complete loss of function/activity. A “hypomorphic” allele is a semi-functional allele caused by a genetic mutation that results in production of the corresponding protein that functions at anywhere between 1% and 99% of normal efficiency.

A “hypermorphic mutation” is a mutation that results in increased expression of the gene product and/or increased activity of the gene product.

As used herein, a “non-natural mutation” refers to a mutation that is generated though human intervention and differs from mutations found in the same gene that have occurred in nature (e.g., occurred naturally)).

A “locus” is a position on a chromosome where a gene or marker or allele is located. In some embodiments, a locus may encompass one or more nucleotides.

As used herein, the terms “desired allele,” “target allele” and/or “allele of interest” are used interchangeably to refer to an allele associated with a desired trait. In some embodiments, a desired allele may be associated with either an increase or a decrease (relative to a control) of or in a given trait, depending on the nature of the desired phenotype.

A marker is “associated with” a trait when said trait is linked to it and when the presence of the marker is an indicator of whether and/or to what extent the desired trait or trait form will occur in a plant/germplasm comprising the marker. Similarly, a marker is “associated with” an allele or chromosome interval when it is linked to it and when the presence of the marker is an indicator of whether the allele or chromosome interval is present in a plant/germplasm comprising the marker.

As used herein, the terms “backcross” and “backcrossing” refer to the process whereby a progeny plant is crossed back to one of its parents one or more times (e.g., 1, 2, 3, 4, 5, 6, 7, 8, etc.). In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. For example, see Ragot, M. et al. Marker-assisted Backcrossing: A Practical Example, in TECHNIQUES ET UTILISATIONS DES MARQUEURS MOLECULAIRES LES COLLOQUES, Vol. 72, pp. 45-56 (1995); and Openshaw et al., Marker-assisted Selection in Backcross Breeding, in PROCEEDINGS OF THE SYMPOSIUM “ANALYSIS OF MOLECULAR MARKER DATA,” pp. 41-43 (1994). The initial cross gives rise to the F1 generation. The term “BC1” refers to the second use of the recurrent parent, “BC2” refers to the third use of the recurrent parent, and so on.

As used herein, the terms “cross” or “crossed” refer to the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

As used herein, the terms “introgression,” “introgressing” and “introgressed” refer to both the natural and artificial transmission of a desired allele or combination of desired alleles of a genetic locus or genetic loci from one genetic background to another. For example, a desired allele at a specified locus can be transmitted to at least one (e.g., one or more) progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele may be a selected allele of a marker, a QTL, a transgene, or the like. Offspring comprising the desired allele can be backcrossed one or more times (e.g., 1, 2, 3, 4, or more times) to a line having a desired genetic background, selecting for the desired allele, with the result being that the desired allele becomes fixed in the desired genetic background. For example, a marker associated with increased yield under non-water stress conditions may be introgressed from a donor into a recurrent parent that does not comprise the marker and does not exhibit increased yield under non-water stress conditions. The resulting offspring could then be backcrossed one or more times and selected until the progeny possess the genetic marker(s) associated with increased yield under non-water stress conditions in the recurrent parent background.

A “genetic map” is a description of genetic linkage relationships among loci on one or more chromosomes within a given species, generally depicted in a diagrammatic or tabular form. For each genetic map, distances between loci are measured by the recombination frequencies between them. Recombination between loci can be detected using a variety of markers. A genetic map is a product of the mapping population, types of markers used, and the polymorphic potential of each marker between different populations. The order and genetic distances between loci can differ from one genetic map to another.

As used herein, the term “genotype” refers to the genetic constitution of an individual (or group of individuals) at one or more genetic loci, as contrasted with the observable and/or detectable and/or manifested trait (the phenotype). Genotype is defined by the allele(s) of one or more known loci that the individual has inherited from its parents. The term genotype can be used to refer to an individual’s genetic constitution at a single locus, at multiple loci, or more generally, the term genotype can be used to refer to an individual’s genetic make-up for all the genes in its genome. Genotypes can be indirectly characterized, e.g., using markers and/or directly characterized by nucleic acid sequencing.

As used herein, the term “germplasm” refers to genetic material of or from an individual (e.g., a plant), a group of individuals (e.g., a plant line, variety or family), or a clone derived from a line, variety, species, or culture. The germplasm can be part of an organism or cell or can be separate from the organism or cell. In general, germplasm provides genetic material with a specific genetic makeup that provides a foundation for some or all of the hereditary qualities of an organism or cell culture. As used herein, germplasm includes cells, seed or tissues from which new plants may be grown, as well as plant parts that can be cultured into a whole plant (e.g., leaves, stems, buds, roots, pollen, cells, etc.).

As used herein, the terms “cultivar” and “variety” refer to a group of similar plants that by structural or genetic features and/or performance can be distinguished from other varieties within the same species.

As used herein, the terms “exotic,” “exotic line” and “exotic germplasm” refer to any plant, line or germplasm that is not elite. In general, exotic plants/germplasms are not derived from any known elite plant or germplasm, but rather are selected to introduce one or more desired genetic elements into a breeding program (e.g., to introduce novel alleles into a breeding program).

As used herein, the term “hybrid” in the context of plant breeding refers to a plant that is the offspring of genetically dissimilar parents produced by crossing plants of different lines or breeds or species, including but not limited to the cross between two inbred lines.

As used herein, the term “inbred” refers to a substantially homozygous plant or variety. The term may refer to a plant or plant variety that is substantially homozygous throughout the entire genome or that is substantially homozygous with respect to a portion of the genome that is of particular interest.

A “haplotype” is the genotype of an individual at a plurality of genetic loci, i.e., a combination of alleles. Typically, the genetic loci that define a haplotype are physically and genetically linked, i.e., on the same chromosome segment. The term “haplotype” can refer to polymorphisms at a particular locus, such as a single marker locus, or polymorphisms at multiple loci along a chromosomal segment.

A plant in which at least one (e.g., one or more, e.g., 1, 2, 3, or 4, or more) endogenous RPL3 gene (e.g., an endogenous RPL3A gene, an endogenous RPL3A-1 gene, an endogenous RPL3A-2 gene, an endogenous RPL3A-3 gene, an endogenous RPL3B gene, an endogenous RPL3B-1 gene, an endogenous RPL3B-2 gene, an endogenous RPL3B-3) is modified as described herein (e.g., comprises a modification as described herein) may result in reduced binding of a trichothecene mycotoxin to the RPL3 polypeptide encoded by the at least one modified endogenous RPL3 gene, may result in increased resistance/tolerance to fusarium head blight (FHB) (e.g., increased resistance/tolerance to the FHB causal agent, Fusarium graminearum, e.g., decreased symptoms of FHB infection, e.g., decreased symptoms of infection by Fusarium graminearum), may result in increased tolerance to trichothecene mycotoxins (e.g., DON), may exhibit a decrease in the amount of Fusarium graminearum present in the plant (e.g., a decreased F. graminearum load), and/or may result in a plant that is capable of producing seed that has a reduced accumulation of trichothecene mycotoxin when infected with Fusarium graminearum as compared to a plant that does not comprise (is devoid of) the modification in the at least one endogenous RPL3 gene.

The trichothecene mycotoxin deoxynivalenol (DON) is believed to act as a virulence factor for F. graminearum by binding to the RPL3 polypeptide of the plant. Without wishing to be limited by any particular theory, by modifying the RPL3 gene such that DON has a reduced ability or no ability to bind the encoded RPL3 polypeptide may reduce the ability of the fungus to infect the plant or part thereof, resulting in a plant or part thereof having increased resistance/tolerance to infection by the fungus and/or a reduced fungal load. Thus, modifications in an endogenous RPL3 gene encoding an RPL3 polypeptide as described herein may regulate the disease response to Fusarium graminearum and/or may provide increased tolerance to mycotoxins produced by F. graminearum, and/or may result in the plant or part thereof comprising a reduced F. graminearum load.

As used herein, an “increased resistance to “FHB” or refers to an increase in resistance to infection by fusarium of about 20% to about 100% (e.g., about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% or more), or any range or value therein, in a plant or part thereof of the invention as compared to a control plant (e.g., a plant not comprising the mutation in an endogenous RPL3 gene as described herein). An increase in resistance may also be referred to as an increase in tolerance to FHB. Such an increase in resistance may be observed as a decrease in plant disease symptoms, optionally without a concomitant change in fungal growth or development in the plant or part thereof. In some embodiments, increased resistance may be measured by a decrease in the symptoms of infection (e.g., a decrease of about 20% to about 100%; e.g., about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%), or any range or value therein, caused by F. graminearum such as a decrease in the disease symptoms of bleaching of infected spikelets in the seed head of the plants. Resistance or tolerance may be measured after infection of spikelets with F. graminearum, or may be measured by exposing the seed head of the plant to a mycotoxin, optionally DON. Thus, an assay for disease may look at spikelets. Spikelets infected with F. graminearum demonstrate premature bleaching and yellowing. Disease resistance may be measured as a decrease in bleaching as compared to a control plant when ranked on a scale from 1 to 9. Disease is typically recorded on a scale and resistance is measured as a relative score that is less than the control. See, e.g., Hales et al. Type II fusarium head blight susceptibility conferred by a region on wheat chromosome 4D J. Exp. Bot. 71 (16):4703-4714 (2020); Hales et al. also disclose that disease severity can be measured based on the area that shows disease symptoms, for example, measuring the number of spikelets that show disease symptoms. In some embodiments, “type II” resistance is measured, and thus, when measuring the number of spikelets that are infected, a decrease by one infected spikelet for a test plant (e.g., an edited plant as described herein) as compared to a control plant indicates that the test plant is more resistant. See also, Wu et al. Linking Multi-Omics to Wheat Resistance Types to Fusarium Head Blight to Reveal the Underlying Mechanisms. Int J Mol Sci. 2022; 23(4):2280. Published 2022 Feb 18. doi:10.3390/ijms23042280.

As used herein, “reduced binding of a trichothecene mycotoxin to the mutated RPL3 polypeptide” refers to a reduction in binding of the trichothecene mycotoxin to the mutated RPL3 by about 15% to about 100% (e.g., about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% or more), or any range or value therein, as compared to a control plant. Reduced binding of the trichothecene mycotoxin to the mutated RPL3 is measured by standard techniques of affinity chromatography as further described in Hassan et al. (Toxins 8:261 (2016)).

As used herein, a “reduced F. graminearum load” or “reduced fungal load” means a reduced amount of F. graminearum present in the plant comprising a mutation (one or more) in at least one endogenous RPL3 gene as compared to a control plant (when exposed to the fungus, F. graminearum). A mutation in an endogenous RPL3 gene as described herein may result in a reduction in the F. graminearum load by about 40% to about 100% (e.g., about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%), or any range or value therein, as compared to a control plant. The F. graminearum load carried in a plant or the amount of F. graminearum in a plant may be measured by, for example, a quantitative PCR technique such as that described by Bakker and McCormick (Phytopathology 109:993-1002 (2019)).

As used herein, the phrase “seed that has a reduced accumulation of trichothecene mycotoxin when infected with Fusarium graminearum” refers to seed in which the level/amount of trichothecene mycotoxin is reduced as compared to seed produced by a plant that does not comprise/is devoid of a mutation (one or more) in at least one endogenous RPL3 gene as described herein. A reduced amount of trichothecene mycotoxin may be about zero to about 3 parts per billion. In some embodiments, the amount of trichothecene mycotoxin may be reduced by about 40% to about 100% (e.g., about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%), or any range or value therein, as compared to a plant that is devoid of the mutation (one or more) in at least one endogenous RPL3 gene.

As used herein, “increased tolerance” to mycotoxins produced by F. graminearum, e.g., increased tolerance to trichothecene mycotoxins (e.g., DON) means a decrease in sensitivity to mycotoxins, e.g., trichothecene mycotoxins (e.g., DON). In some embodiments, a plant or part thereof of the invention comprising one or more mutations in an endogenous RPL3 gene as described herein may exhibit an increased tolerance/decreased sensitivity to trichothecene mycotoxins (e.g., DON) of about 10% to about 100% (e.g., about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% or more), or any range or value therein, as compared to a plant that is devoid of the mutation (one or more) in at least one endogenous RPL3 gene. In some embodiments, tolerance to a trichothecene mycotoxin may be measured using spikelet bleaching and yellowing as described above.

As used herein a “control plant” means a plant that does not contain an edited RPL3 gene or gene as described herein that imparts an enhanced/improved trait (e.g., yield trait) or altered phenotype. A control plant is used to identify and select a plant edited as described herein and that has an enhanced trait or altered phenotype as compared to the control plant. A suitable control plant can be a plant of the parental line used to generate a plant comprising a mutated RPL3 gene(s), for example, a wild type plant devoid of an edit in an endogenous RPL3 gene as described herein. A suitable control plant can also be a plant that contains recombinant nucleic acids that impart other traits, for example, a transgenic plant having enhanced herbicide tolerance. A suitable control plant can in some cases be a progeny of a heterozygous or hemizygous transgenic plant line that is devoid of the mutated RPL3 gene as described herein, known as a negative segregant, or a negative isogenic line.

An enhanced trait (e.g., improved yield trait) may include, for example, decreased days from planting to maturity, increased stalk size, increased number of leaves, increased plant height growth rate in vegetative stage, increased ear size, increased ear dry weight per plant, increased number of kernels per ear, increased weight per kernel, increased number of kernels per plant, decreased ear void, extended grain fill period, reduced plant height, increased number of root branches, increased total root length, increased yield, increased nitrogen use efficiency, and/or increased water use efficiency as compared to a control plant. An altered phenotype may be, for example, plant height, biomass, canopy area, anthocyanin content, chlorophyll content, water applied, water content, and water use efficiency.

In some embodiments, a plant of this invention may comprise one or more improved yield traits including, but not limited to, In some embodiments, the one or more improved yield traits includes higher yield (bu/acre), increased biomass, increased plant height, increased stem diameter, increased leaf area, increased number of flowers, increased kernel row number, optionally wherein ear length is not substantially reduced, increased kernel number, increased kernel size, increased ear length, decreased tiller number, decreased tassel branch number, increased number of pods, including an increased number of pods per node and/or an increased number of pods per plant, increased number of seeds per pod, increased number of seeds, increased seed size, and/or increased seed weight (e.g., increase in 100-seed weight) as compared to a control plant devoid of the mutation in the RPL3 gene as described herein. In some embodiments, a plant of this invention may comprise one or more improved yield traits including, but not limited to, optionally an increase in yield (bu/acre), seed size (including kernel size), seed weight (including kernel weight), increased kernel row number (optionally wherein ear length is not substantially reduced), increased number of pods, increased number of seeds per pod and an increase in ear length as compared to a control plant or part thereof.

As used herein a “trait” is a physiological, morphological, biochemical, or physical characteristic of a plant or particular plant material or cell. In some instances, this characteristic is visible to the human eye and can be measured mechanically, such as seed or plant size, weight, shape, form, length, height, growth rate and development stage, or can be measured by biochemical techniques, such as detecting the protein, starch, certain metabolites, or oil content of seed or leaves, or by observation of a metabolic or physiological process, for example, by measuring tolerance to water deprivation or particular salt or sugar concentrations, or by the measurement of the expression level of a gene or genes, for example, by employing Northern analysis, RT-PCR, microarray gene expression assays, or reporter gene expression systems, or by agricultural observations such as hyperosmotic stress tolerance or yield. However, any technique can be used to measure the amount of, the comparative level of, or the difference in any selected chemical compound or macromolecule in the transgenic plants.

As used herein an “enhanced trait” means a characteristic of a plant resulting from mutations in a RPL3 gene(s) as described herein. Such traits include, but are not limited to, an enhanced agronomic trait characterized by enhanced plant morphology, physiology, growth and development, yield, nutritional enhancement, disease or pest resistance, or environmental or chemical tolerance. In some embodiments, an enhanced trait/altered phenotype may be, for example, decreased days from planting to maturity, increased stalk size, increased number of leaves, increased plant height growth rate in vegetative stage, increased ear size, increased ear dry weight per plant, increased number of kernels per ear, increased weight per kernel, increased number of kernels per plant, decreased ear void, extended grain fill period, reduced plant height, increased number of root branches, increased total root length, drought tolerance, increased water use efficiency, cold tolerance, increased nitrogen use efficiency, and/or increased yield. In some embodiments, a trait is increased yield under nonstress conditions or increased yield under environmental stress conditions. Stress conditions can include both biotic and abiotic stress, for example, drought, shade, fungal disease, viral disease, bacterial disease, insect infestation, nematode infestation, cold temperature exposure, heat exposure, osmotic stress, reduced nitrogen nutrient availability, reduced phosphorus nutrient availability and high plant density. “Yield” can be affected by many properties including without limitation, plant height, plant biomass, pod number, pod position on the plant, number of internodes, incidence of pod shatter, grain size, ear size, ear tip filling, kernel abortion, efficiency of nodulation and nitrogen fixation, efficiency of nutrient assimilation, resistance to biotic and abiotic stress, carbon assimilation, plant architecture, resistance to lodging, percent seed germination, seedling vigor, and juvenile traits. Yield can also be affected by efficiency of germination (including germination in stressed conditions), growth rate (including growth rate in stressed conditions), flowering time and duration, ear number, ear size, ear weight, seed number per ear or pod, seed size, composition of seed (starch, oil, protein) and characteristics of seed fill.

Also used herein, the term “trait modification” encompasses altering the naturally occurring trait by producing a detectable difference in a characteristic in a plant comprising a mutation in an endogenous RPL3 gene as described herein relative to a plant not comprising the mutation, such as a wild-type plant, or a negative segregant. In some cases, the trait modification can be evaluated quantitatively. For example, the trait modification can entail an increase or decrease in an observed trait characteristic or phenotype as compared to a control plant. It is known that there can be natural variations in a modified trait. Therefore, the trait modification observed can entail a change of the normal distribution and magnitude of the trait characteristics or phenotype in the plants as compared to a control plant.

The present disclosure relates to a plant with improved economically relevant characteristics, more specifically plants having increased resistance or tolerance to Fusarium head blight (FHB) (e.g., increased resistance/tolerance to the FHB causal agent, Fusarium graminearum e.g., decreased symptoms FHB infection, e.g., decreased symptoms of infection following infection by Fusarium graminearum), decreased F. graminearum load, decreased DON accumulation following infection by Fusarium graminearum, increased tolerance to a trichothecene mycotoxin, e.g., deoxynivalenol (DON) and/or plants having the ability to produce seed with a reduced accumulation of trichothecene mycotoxin when plant bearing the seeds is infected with Fusarium graminearum. In some embodiments, a plant of this invention may further comprise a RPL3 gene modified as described herein that produces a modified RPL3 polypeptide having reduced binding to trichothecene mycotoxins. In some embodiments, a plant of this invention comprises a RPL3 gene modified as described herein that produced no detectable RPL3 polypeptide or a truncated RPL3 polypeptide. In some embodiments, the plant may further exhibit improved yield characteristics and/or an improved plant architecture (which can contribute to improved yield traits). More specifically the present disclosure relates to a plant comprising a mutation(s) in a RPL3 gene(s) as described herein, wherein the plant has increased resistance or tolerance to Fusarium head blight (FHB) (e.g., decreased symptoms of FHB) and/or increased tolerance to a trichothecene mycotoxin, and optionally increased yield as compared to a control plant devoid of said mutation(s). In some embodiments, a plant of the present disclosure exhibits an improved trait that is related to yield, including but not limited to increased nitrogen use efficiency, increased nitrogen stress tolerance, increased water use efficiency and/or increased drought tolerance, as defined and discussed infra.

Yield can be defined as the measurable produce of economic value from a crop. Yield can be defined in the scope of quantity and/or quality. Yield can be directly dependent on several factors, for example, the number and size of organs (e.g., number of flowers), plant architecture (such as the number of branches, plant biomass, e.g., increased root biomass, steeper root angle and/or longer roots, and the like), flowering time and duration, grain fill period. Root architecture and development, photosynthetic efficiency, nutrient uptake, stress tolerance, early vigor, delayed senescence and functional stay green phenotypes may be factors in determining yield. Optimizing the above-mentioned factors can therefore contribute to increasing crop yield.

Reference herein to an increase/improvement in yield-related traits can also be taken to mean an increase in biomass (weight) of one or more parts of a plant, which can include above ground and/or below ground (harvestable) plant parts. In particular, such harvestable parts are seeds, and performance of the methods of the disclosure results in plants with increased yield and in particular increased seed yield relative to the seed yield of suitable control plants. The term “yield” of a plant can relate to vegetative biomass (root and/or shoot biomass), to reproductive organs, and/or to propagules (such as seeds) of that plant.

Increased yield of a plant of the present disclosure can be measured in a number of ways, including test weight, seed number per plant, seed weight, seed number per unit area (for example, seeds, or weight of seeds, per acre), bushels per acre, tons per acre, or kilo per hectare. Increased yield can result from improved utilization of key biochemical compounds, such as nitrogen, phosphorous and carbohydrate, or from improved responses to environmental stresses, such as cold, heat, drought, salt, shade, high plant density, and attack by pests or pathogens.

“Increased yield” can manifest as one or more of the following: (i) increased plant biomass (weight) of one or more parts of a plant, particularly aboveground (harvestable) parts, of a plant, increased root biomass (increased number of roots, increased root thickness, increased root length) or increased biomass of any other harvestable part; or (ii) increased early vigor, defined herein as an improved seedling aboveground area approximately three weeks post-germination.

“Early vigor” refers to active healthy plant growth especially during early stages of plant growth, and can result from increased plant fitness due to, for example, the plants being better adapted to their environment (for example, optimizing the use of energy resources, uptake of nutrients and partitioning carbon allocation between shoot and root). Early vigor, for example, can be a combination of the ability of seeds to germinate and emerge after planting and the ability of the young plants to grow and develop after emergence. Plants having early vigor also show increased seedling survival and better establishment of the crop, which often results in highly uniform fields with the majority of the plants reaching the various stages of development at substantially the same time, which often results in increased yield. Therefore, early vigor can be determined by measuring various factors, such as kernel weight, percentage germination, percentage emergence, seedling growth, seedling height, root length, root and shoot biomass, canopy size and color and others.

Further, increased yield can also manifest as increased total seed yield, which may result from one or more of an increase in seed biomass (seed weight) due to an increase in the seed weight on a per plant and/or on an individual seed basis an increased number of, for example, flowers/panicles per plant; an increased number of pods; an increased number of nodes; an increased number of flowers (“florets”) per panicle/plant; increased seed fill rate; an increased number of filled seeds; increased seed size (length, width, area, perimeter, and/or weight), which can also influence the composition of seeds; and/or increased seed volume, which can also influence the composition of seeds. In one embodiment, increased yield can be increased seed yield, for example, increased seed weight; increased number of filled seeds; and/or increased harvest index.

Increased yield can also result in modified architecture, or can occur because of modified plant architecture.

Increased yield can also manifest as increased harvest index, which is expressed as a ratio of the yield of harvestable parts, such as seeds, over the total biomass

The disclosure also extends to harvestable parts of a plant such as, but not limited to, seeds, leaves, fruits, flowers, bolls, pods, siliques, nuts, stems, rhizomes, tubers and bulbs. The disclosure furthermore relates to products derived from a harvestable part of such a plant, such as dry pellets, powders, oil, fat and fatty acids, starch or proteins.

The present disclosure provides a method for increasing “yield” of a plant or “broad acre yield” of a plant or plant part defined as the harvestable plant parts per unit area, for example seeds, or weight of seeds, per acre, pounds per acre, bushels per acre, tones per acre, tons per acre, kilo per hectare.

As used herein “nitrogen use efficiency” refers to the processes which lead to an increase in the plant’s yield, biomass, vigor, and growth rate per nitrogen unit applied. The processes can include the uptake, assimilation, accumulation, signaling, sensing, retranslocation (within the plant) and use of nitrogen by the plant.

As used herein “increased nitrogen use efficiency” refers to the ability of plants to grow, develop, or yield faster or better than normal when subjected to the same amount of available/applied nitrogen as under normal or standard conditions; ability of plants to grow, develop, or yield normally, or grow, develop, or yield faster or better when subjected to less than optimal amounts of available/applied nitrogen, or under nitrogen limiting conditions.

As used herein “nitrogen limiting conditions” refers to growth conditions or environments that provide less than optimal amounts of nitrogen needed for adequate or successful plant metabolism, growth, reproductive success and/or viability.

As used herein the “increased nitrogen stress tolerance” refers to the ability of plants to grow, develop, or yield normally, or grow, develop, or yield faster or better when subjected to less than optimal amounts of available/applied nitrogen, or under nitrogen limiting conditions.

Increased plant nitrogen use efficiency can be translated in the field into either harvesting similar quantities of yield, while supplying less nitrogen, or increased yield gained by supplying optimal/sufficient amounts of nitrogen. The increased nitrogen use efficiency can improve plant nitrogen stress tolerance and can also improve crop quality and biochemical constituents of the seed such as protein yield and oil yield. The terms “increased nitrogen use efficiency”, “enhanced nitrogen use efficiency”, and “nitrogen stress tolerance” are used inter-changeably in the present disclosure to refer to plants with improved productivity under nitrogen limiting conditions.

As used herein “water use efficiency” refers to the amount of carbon dioxide assimilated by leaves per unit of water vapor transpired. It constitutes one of the most important traits controlling plant productivity in dry environments. “Drought tolerance” refers to the degree to which a plant is adapted to arid or drought conditions. The physiological responses of plants to a deficit of water include leaf wilting, a reduction in leaf area, leaf abscission, and the stimulation of root growth by directing nutrients to the underground parts of the plants. Typically, plants are more susceptible to drought during flowering and seed development (the reproductive stages), as plant’s resources are deviated to support root growth. In addition, abscisic acid (ABA), a plant stress hormone, induces the closure of leaf stomata (microscopic pores involved in gas exchange), thereby reducing water loss through transpiration, and decreasing the rate of photosynthesis. These responses improve the water-use efficiency of the plant on the short term. The terms “increased water use efficiency”, “enhanced water use efficiency”, and “increased drought tolerance” are used inter-changeably in the present disclosure to refer to plants with improved productivity under water-limiting conditions.

As used herein “increased water use efficiency” refers to the ability of plants to grow, develop, or yield faster or better than normal when subjected to the same amount of available/applied water as under normal or standard conditions; ability of plants to grow, develop, or yield normally, or grow, develop, or yield faster or better when subjected to reduced amounts of available/applied water (water input) or under conditions of water stress or water deficit stress.

As used herein “increased drought tolerance” refers to the ability of plants to grow, develop, or yield normally, or grow, develop, or yield faster or better than normal when subjected to reduced amounts of available/applied water and/or under conditions of acute or chronic drought; ability of plants to grow, develop, or yield normally when subjected to reduced amounts of available/applied water (water input) or under conditions of water deficit stress or under conditions of acute or chronic drought.

As used herein, “drought stress” refers to a period of dryness (acute or chronic/prolonged) that results in water deficit and subjects plants to stress and/or damage to plant tissues and/or negatively affects grain/crop yield; a period of dryness (acute or chronic/prolonged) that results in water deficit and/or higher temperatures and subjects plants to stress and/or damage to plant tissues and/or negatively affects grain/crop yield.

As used herein, “water deficit” refers to the conditions or environments that provide less than optimal amounts of water needed for adequate/successful growth and development of plants.

As used herein, “water stress” refers to the conditions or environments that provide improper (either less/insufficient or more/excessive) amounts of water than that needed for adequate/successful growth and development of plants/crops thereby subjecting the plants to stress and/or damage to plant tissues and/or negatively affecting grain/crop yield.

As used herein “water deficit stress” refers to the conditions or environments that provide less/insufficient amounts of water than that needed for adequate/successful growth and development of plants/crops thereby subjecting the plants to stress and/or damage to plant tissues and/or negatively affecting grain yield.

As used herein, the terms “nucleic acid,” “nucleic acid molecule,” “nucleotide sequence” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment or portion, genomic DNA, synthetic (e.g., chemically synthesized) DNA, plasmid DNA, mRNA, and anti-sense RNA, any of which can be single stranded or double stranded. The terms “nucleotide sequence” “nucleic acid,” “nucleic acid molecule,” “nucleic acid construct,” “oligonucleotide” and “polynucleotide” are also used interchangeably herein to refer to a heteropolymer of nucleotides. Nucleic acid molecules and/or nucleotide sequences provided herein are presented herein in the 5′ to 3′ direction, from left to right and are represented using the standard code for representing the nucleotide characters as set forth in the U.S. sequence rules, 37 CFR §§1.821 - 1.825 and the World Intellectual Property Organization (WIPO) Standard ST.25. A “5′ region” as used herein can mean the region of a polynucleotide that is nearest the 5′ end of the polynucleotide. Thus, for example, an element in the 5′ region of a polynucleotide can be located anywhere from the first nucleotide located at the 5′ end of the polynucleotide to the nucleotide located halfway through the polynucleotide. A “3′ region” as used herein can mean the region of a polynucleotide that is nearest the 3′ end of the polynucleotide. Thus, for example, an element in the 3′ region of a polynucleotide can be located anywhere from the first nucleotide located at the 3′ end of the polynucleotide to the nucleotide located halfway through the polynucleotide.

As used herein with respect to nucleic acids, the term “fragment” or “portion” refers to a nucleic acid that is reduced in length relative (e.g., reduced by 1, 2, 3, 4, 5, 6, 7, 8, 9, 10, 11, 12, 13, 14, 15, 16, 17, 18, 19, 20, 20, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 400, 450, 500, 550, 600, 650, 700, 750, 800, 850, or 900 or more nucleotides or any range or value therein) to a reference nucleic acid and that comprises, consists essentially of and/or consists of a nucleotide sequence of contiguous nucleotides identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to a corresponding portion of the reference nucleic acid. Such a nucleic acid fragment may be, where appropriate, included in a larger polynucleotide of which it is a constituent. As an example, a repeat sequence of guide nucleic acid of this invention may comprise a “portion” of a wild type CRISPR-Cas repeat sequence (e.g., a wild type CRISPR-Cas repeat; e.g., a repeat from the CRISPR Cas system of, for example, a Cas9, Cas12a (Cpf1), Cas12b, Cas12c (C2c3), Cas12d (CasY), Cas12e (CasX), Cas12g, Cas12h, Cas12i, C2c4, C2c5, C2c8, C2c9, C2c10, Cas14a, Cas14b, and/or a Cas14c, and the like).

In some embodiments, a nucleic acid fragment may comprise, consist essentially of or consist of about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 320, 330, 340, 350, 360, 370, 380, 390, 395, 400, 410, 415, 420, 425, 430, 435, 440, 445, 450, 500, 550, 600, 650, 700, 750, 800, 850, 900, 950, 1000, 1100, 1150, 1200, 1250, 1300, 1350, 1400, 1450, 1500, 1550, 1600, 1650, 1700, 1750, 1800, 1900, 2000, 2100, 2200, 2300, 2400, or 2500 or more consecutive nucleotides, or any range or value therein, of a nucleic acid encoding a RPL3 polypeptide, optionally a fragment of a RPL3 gene may be about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 115, 120, 125, 130, 135, 140, 145, 150 consecutive nucleotides to about 155, 160, 165, 170, 175, 180, 185, 190, 195, 200, 205, 210, 215, 220, 225, 230, 240, 245, 250, 255, 260, 265, 270, 275, 280, 285, 290, 295, 300, 305, 310, 315, 320, 325, 330, 340, 345, 350, 355, 360, 365, 370, 375, 380, 385, 390, 395, or 400, or more consecutive nucleotides in length, or any range or value therein (e.g., a fragment or portion of any one of SEQ ID NO:72, 73, 75, 76, 78, 79, 80, 81, 83, 84, 86, or 87 (e.g., any one of SEQ ID NOs:89-139).

In some embodiments, a “sequence-specific nucleic acid binding domain” may bind to one or more fragments or portions of nucleotide sequences (e.g., DNA, RNA) encoding, for example, a Ribosomal Protein L3 (RPL3) polypeptide as described herein.

As used herein with respect to polypeptides, the term “fragment” or “portion” may refer to a polypeptide that is reduced in length relative to a reference polypeptide and that comprises, consists essentially of and/or consists of an amino acid sequence of contiguous amino acids identical or almost identical (e.g., 90%, 91%, 92%, 93%, 94%, 95%, 96%, 97%, 98%, 99% identical) to a corresponding portion of the reference polypeptide. Such a polypeptide fragment may be, where appropriate, included in a larger polypeptide of which it is a constituent. In some embodiments, a polypeptide fragment may comprise, consist essentially of, or consist of at least about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 260, 270, 280, or 290 or more consecutive amino acids of a reference polypeptide. In some embodiments, a polypeptide fragment may comprise, consist essentially of or consist of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 230, 240, or 250 or more consecutive amino acid residues, or any range or value therein, of a RPL3 polypeptide (e.g., a fragment or a portion of any one of SEQ ID NO:74, 77, 82, 85, or 88). In some embodiments, a fragment of a RPL3 polypeptide can be the N-terminus of the polypeptide or a portion thereof (see, e.g., a sequence of consecutive amino acids from about residue 10 to about residue 200 of any one of SEQ ID NO:74, 77, 82, 85, or 88). In some embodiments, a fragment of a RPL3 polypeptide may be the result of a mutation generated in at least one endogenous gene encoding a RPL3 polypeptide (e.g., a RPL31gene and/or a RPL3B gene) as described herein (e.g., a deletion, insertion and the like, in one or more of the endogenous RPL3 genes in a plant).

In some embodiments, a mutation generated in at least one endogenous gene encoding a RPL3 polypeptide when comprised in a plant can result in the plant having an RPL3 polypeptide with reduced binding to a trichothecene mycotoxin (e.g., DON) as compared to a plant not comprising said deletion. In some embodiments, the plant comprising a mutation in an endogenous RPL3 gene as described herein may exhibit increased tolerance to trichothecene mycotoxin, may exhibit increased resistance/tolerance to Fusarium head blight (FHB) (e.g., increased resistance/tolerance to the causal agent of FHB, Fusarium graminearum, e.g., decreased symptoms FHB infection, e.g., decreased symptoms of infection by Fusarium graminearum), and/or may exhibit/carry a decreased Fusarium graminearum load, e.g., a decreased level/amount of F. graminearum, as compared to a plant not comprising said mutation. A RPL3 gene may be edited in one or more than one location (and using one or more different editing tools), thereby providing a RPL3 gene comprising one or more than one mutation. In some embodiments, a RPL3 polypeptide mutated as described herein may comprise one or more than one edit that may result in a polypeptide having a deletion of one or more amino acid residues (e.g., a deletion of 1, 2, 3, 4, 5, 6, 7, 8, 9, 10, 20, 30, 40, 50, 60, 70, 80, 90, 100, 150, 200, 250, 300, 350, or 380 or more consecutive amino acid residues, and any range or value therein (e.g., a truncated polypeptide), optionally a deletion of about 10 to about 200 consecutive amino acid residues (e.g., about 10, 20, 30, 40, 50, 60, 70, 80, 90, 100, 110, 120, 130, 140, 150, 160, 170, 180, 190, or 200, and any range or value therein).

In some embodiments, a “portion” or “region” in reference to a nucleic acid means at least 2, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135,140, 145, 150, 155, 160, 165, 170, 175, 180, 185, 190, 195, 200, 210, 220, 230, 240, 250, 260, 270, 280, 285, 290, 300, 310, 320, 330, 340, 350, 360, 370, 380, 390, 395, 400, 405, 410, 415, 420, 425, 430, 435, 440, 445, 450, 500, 600, 700, 800, 900, 1000, 1100, 1200, 1300, 1400, 1500, 1600, 1700, 1800, 1900, 2000, 2100, 2200, 2300, 2400, or 2500 or more consecutive nucleotides from a gene (e.g., consecutive nucleotides from a RPL3 gene), optionally a “portion” or “region” of a RPL3 gene may be about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, or 150 consecutive nucleotides to about 155, 160, 165, 170, 175, 180, 185, 190, 195, 200, 205, 210, 215, 220, 225, 230, 240, 245, 250, 255, 260, 265, 270, 275, 280, 285, 290, 295, 300, 305, 310, 315, 320, 325, 330, 340, 345, 350, 355, 356, 357, 358, or 359 or more consecutive nucleotides in length, or any range or value therein (e.g., a portion or region of any one of SEQ ID NO:72, 73, 75, 76, 78, 79, 80, 81, 83, 84, 86, or 87 (e.g., any one of SEQ ID NOs:89-139).

In some embodiments, a “portion” or “region” of a RPL3 polypeptide sequence may be about 5 to about 225 or more consecutive amino acid residues in length (e.g., about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 1699, 200, 201, 202, 203, 204, 205, 206, 207, 208, 209, 210, 211, 212, 213, 214, 215, 216, 217, 218, 219,220, 221, 222, 223, 224, or 225, or more consecutive amino acid residues in length (e.g., a portion of any one of SEQ ID NO:74, 77, 82, 85, or 88, optionally the portion or region is from the N-terminal end of a RPL3 polypeptide, e.g., a sequence comprising consecutive amino acid residues from about residue 10 to about residue 225 of any one of SEQ ID NO:74, 77, 82, 85, or 88.

As used herein with respect to nucleic acids, the term “functional fragment” refers to nucleic acid that encodes a functional fragment of a polypeptide. A “functional fragment” with respect to a polypeptide is a fragment of a polypeptide that retains one or more of the activities of the native reference polypeptide.

The term “gene,” as used herein, refers to a nucleic acid molecule capable of being used to produce mRNA, antisense RNA, miRNA, anti-microRNA antisense oligodeoxyribonucleotide (AMO) and the like. Genes may or may not be capable of being used to produce a functional protein or gene product. Genes can include both coding and non-coding regions (e.g., introns, regulatory elements, promoters, enhancers, termination sequences and/or 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

The term “mutation” refers to point mutations (e.g., missense, or nonsense, or insertions or deletions of single base pairs that result in frame shifts), insertions, deletions, inversions and/or truncations. When the mutation is a substitution of a residue within an amino acid sequence with another residue, or a deletion or insertion of one or more residues within a sequence, the mutations are typically described by identifying the original residue followed by the position of the residue within the sequence and by the identity of the newly substituted residue. A truncation can include a truncation at the C-terminal end of a polypeptide or at the N-terminal end of a polypeptide. A truncation of a polypeptide can be the result of a deletion of the corresponding 5′ end or 3′ end of the gene encoding the polypeptide. A frameshift mutation can occur when deletions or insertions of one or more base pairs are introduced into a gene, optionally resulting in an out-of-frame mutation or an in-frame mutation. Frameshift mutations in a gene can result in the production of a polypeptide that is longer, shorter or the same length as the wild type polypeptide depending on when the first stop codon occurs following the mutated region of the gene. As an example, an out-of-frame mutation that produces a premature stop codon can produce a polypeptide that is shorter that the wild type polypeptide, or, in some embodiments, the polypeptide may be absent/undetectable. A DNA inversion is the result of a rotation of a genetic fragment within a region of a chromosome.

The terms “complementary” or “complementarity,” as used herein, refer to the natural binding of polynucleotides under permissive salt and temperature conditions by base-pairing. For example, the sequence “A-G-T” (5′ to 3′) binds to the complementary sequence “T-C-A” (3′ to 5′). Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

“Complement,” as used herein, can mean 100% complementarity with the comparator nucleotide sequence or it can mean less than 100% complementarity (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like, complementarity) to the comparator nucleotide sequence.

Different nucleic acids or proteins having homology are referred to herein as “homologues.” The term homologue includes homologous sequences from the same and from other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the compositions and methods of the invention further comprise homologues to the nucleotide sequences and polypeptide sequences of this invention. “Orthologous,” as used herein, refers to homologous nucleotide sequences and/ or amino acid sequences in different species that arose from a common ancestral gene during speciation. A homologue of a nucleotide sequence of this invention has a substantial sequence identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to said nucleotide sequence of the invention.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent sequence identity” can refer to the percentage of identical amino acids in an amino acid sequence as compared to a reference polypeptide.

As used herein, the phrase “substantially identical,” or “substantial identity” in the context of two nucleic acid molecules, nucleotide sequences, or polypeptide sequences, refers to two or more sequences or subsequences that have at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% nucleotide or amino acid residue identity, when compared and aligned for maximum correspondence, as measured using one of the following sequence comparison algorithms or by visual inspection. In some embodiments of the invention, the substantial identity exists over a region of consecutive nucleotides of a nucleotide sequence of the invention that is about 10 nucleotides to about 20 nucleotides, about 10 nucleotides to about 25 nucleotides, about 10 nucleotides to about 30 nucleotides, about 15 nucleotides to about 25 nucleotides, about 30 nucleotides to about 40 nucleotides, about 50 nucleotides to about 60 nucleotides, about 70 nucleotides to about 80 nucleotides, about 90 nucleotides to about 100 nucleotides, about 100 nucleotides to about 200 nucleotides, about 100 nucleotides to about 300 nucleotides, about 100 nucleotides to about 400 nucleotides, about 100 nucleotides to about 500 nucleotides, about 100 nucleotides to about 600 nucleotides, about 100 nucleotides to about 800 nucleotides, about 100 nucleotides to about 900 nucleotides, or more in length, or any range therein, up to the full length of the sequence. In some embodiments, nucleotide sequences can be substantially identical over at least about 20 nucleotides (e.g., about 20, 21, 22, 23, 24, 25, 26, 27, 28, 29, 30, 31, 32, 33, 34, 35, 36, 37, 38, 39, 40, 50, 60, 70, or 80 nucleotides or more).

In some embodiments of the invention, the substantial identity exists over a region of consecutive amino acid residues of a polypeptide of the invention that is about 3 amino acid residues to about 20 amino acid residues, about 5 amino acid residues to about 25 amino acid residues, about 7 amino acid residues to about 30 amino acid residues, about 10 amino acid residues to about 25 amino acid residues, about 15 amino acid residues to about 30 amino acid residues, about 20 amino acid residues to about 40 amino acid residues, about 25 amino acid residues to about 40 amino acid residues, about 25 amino acid residues to about 50 amino acid residues, about 30 amino acid residues to about 50 amino acid residues, about 40 amino acid residues to about 50 amino acid residues, about 40 amino acid residues to about 70 amino acid residues, about 50 amino acid residues to about 70 amino acid residues, about 60 amino acid residues to about 80 amino acid residues, about 70 amino acid residues to about 80 amino acid residues, about 90 amino acid residues to about 100 amino acid residues, or more amino acid residues in length, and any range therein, up to the full length of the sequence. In some embodiments, polypeptide sequences can be substantially identical to one another over at least about 8 consecutive amino acid residues (e.g., about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 130, 140, 150, 175, 200, 225, 250, 300, 350 or more amino acids in length or more consecutive amino acid residues). In some embodiments, two or more RPL3 polypeptides may be identical or substantially identical (e.g., at least 70% to 99.9% identical, e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or 99.9% identical or any range or value therein) over at least 8 consecutive amino acids to about 350 consecutive amino acids. In some embodiments, two or more RPL3 polypeptides may be identical or substantially identical over at least 8, 9, 10, 11, 12, 13, 14, or 15 consecutive amino acids to about 20, 21, 22, 23, 24, 25, 26, 27, 28, 29, 30, 31, 32, 33, 34, 35, 36, 37, 38, 39, or 40 consecutive amino acids).

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, CA). An “identity fraction” for aligned segments of a test sequence and a reference sequence is the number of identical components which are shared by the two aligned sequences divided by the total number of components in the reference sequence segment, e.g., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Two nucleotide sequences may also be considered substantially complementary when the two sequences hybridize to each other under stringent conditions. In some embodiments, two nucleotide sequences considered to be substantially complementary hybridize to each other under highly stringent conditions.

“Stringent hybridization conditions” and “stringent hybridization wash conditions” in the context of nucleic acid hybridization experiments such as Southern and Northern hybridizations are sequence dependent and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleotide sequences which have more than 100 complementary residues on a filter in a Southern or northern blot is 50% formamide with 1 mg of heparin at 42° C., with the hybridization being carried out overnight. An example of highly stringent wash conditions is 0.1 5 M NaCl at 72° C. for about 15 minutes. An example of stringent wash conditions is a 0.2x SSC wash at 65° C. for 15 minutes (see, Sambrook, infra, for a description of SSC buffer). Often, a high stringency wash is preceded by a low stringency wash to remove background probe signal. An example of a medium stringency wash for a duplex of, e.g., more than 100 nucleotides, is 1x SSC at 45° C. for 15 minutes. An example of a low stringency wash for a duplex of, e.g., more than 100 nucleotides, is 4-6x SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2x (or higher) than that observed for an unrelated probe in the particular hybridization assay indicates detection of a specific hybridization. Nucleotide sequences that do not hybridize to each other under stringent conditions are still substantially identical if the proteins that they encode are substantially identical. This can occur, for example, when a copy of a nucleotide sequence is created using the maximum codon degeneracy permitted by the genetic code.

A polynucleotide and/or recombinant nucleic acid construct of this invention (e.g., expression cassettes and/or vectors) may be codon optimized for expression. In some embodiments, the polynucleotides, nucleic acid constructs, expression cassettes, and/or vectors of the editing systems of the invention (e.g., comprising/encoding a sequence-specific nucleic acid binding domain (e.g., a sequence-specific nucleic acid binding domain (e.g., DNA binding domain) from a polynucleotide-guided endonuclease, a zinc finger nuclease, a transcription activator-like effector nuclease (TALEN), an Argonaute protein, and/or a CRISPR-Cas endonuclease (e.g., CRISPR-Cas effector protein) (e.g., a Type I CRISPR-Cas effector protein, a Type II CRISPR-Cas effector protein, a Type III CRISPR-Cas effector protein, a Type IV CRISPR-Cas effector protein, a Type V CRISPR-Cas effector protein or a Type VI CRISPR-Cas effector protein)), a nuclease (e.g., an endonuclease (e.g., Fok1), a polynucleotide-guided endonuclease, a CRISPR-Cas endonuclease (e.g., CRISPR-Cas effector protein), a zinc finger nuclease, and/or a transcription activator-like effector nuclease (TALEN)), deaminase proteins/domains (e.g., adenine deaminase, cytosine deaminase), a polynucleotide encoding a reverse transcriptase protein or domain, a polynucleotide encoding a 5′-3′ exonuclease polypeptide, and/or affinity polypeptides, peptide tags, etc.) may be codon optimized for expression in a plant. In some embodiments, the codon optimized nucleic acids, polynucleotides, expression cassettes, and/or vectors of the invention have about 70% to about 99.9% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%. 99.9% or 100%) identity or more to the reference nucleic acids, polynucleotides, expression cassettes, and/or vectors that have not been codon optimized.

In any of the embodiments described herein, a polynucleotide or nucleic acid construct of the invention may be operatively associated with a variety of promoters and/or other regulatory elements for expression in a plant and/or a cell of a plant. Thus, in some embodiments, a polynucleotide or nucleic acid construct of this invention may further comprise one or more promoters, introns, enhancers, and/or terminators operably linked to one or more nucleotide sequences. In some embodiments, a promoter may be operably associated with an intron (e.g., Ubi1 promoter and intron). In some embodiments, a promoter associated with an intron maybe referred to as a “promoter region” (e.g., Ubi1 promoter and intron).

By “operably linked” or “operably associated” as used herein in reference to polynucleotides, it is meant that the indicated elements are functionally related to each other and are also generally physically related. Thus, the term “operably linked” or “operably associated” as used herein, refers to nucleotide sequences on a single nucleic acid molecule that are functionally associated. Thus, a first nucleotide sequence that is operably linked to a second nucleotide sequence means a situation when the first nucleotide sequence is placed in a functional relationship with the second nucleotide sequence. For instance, a promoter is operably associated with a nucleotide sequence if the promoter effects the transcription or expression of said nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, nucleic acid sequences can be present between a promoter and the nucleotide sequence, and the promoter can still be considered “operably linked” to the nucleotide sequence.

As used herein, the term “linked,” in reference to polypeptides, refers to the attachment of one polypeptide to another. A polypeptide may be linked to another polypeptide (at the N-terminus or the C-terminus) directly (e.g., via a peptide bond) or through a linker.

The term “linker” is art-recognized and refers to a chemical group, or a molecule linking two molecules or moieties, e.g., two domains of a fusion protein, such as, for example, a nucleic acid binding polypeptide or domain and peptide tag and/or a reverse transcriptase and an affinity polypeptide that binds to the peptide tag; or a DNA endonuclease polypeptide or domain and peptide tag and/or a reverse transcriptase and an affinity polypeptide that binds to the peptide tag. A linker may be comprised of a single linking molecule or may comprise more than one linking molecule. In some embodiments, the linker can be an organic molecule, group, polymer, or chemical moiety such as a bivalent organic moiety. In some embodiments, the linker may be an amino acid or it may be a peptide. In some embodiments, the linker is a peptide.

In some embodiments, a peptide linker useful with this invention may be about 2 to about 100 or more amino acids in length, for example, about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length (e.g., about 2 to about 40, about 2 to about 50, about 2 to about 60, about 4 to about 40, about 4 to about 50, about 4 to about 60, about 5 to about 40, about 5 to about 50, about 5 to about 60, about 9 to about 40, about 9 to about 50, about 9 to about 60, about 10 to about 40, about 10 to about 50, about 10 to about 60, or about 2, 3, 4, 5, 6, 7, 8, 9, 10, 11, 12, 13, 14, 15, 16, 17, 18, 19, 20, 21, 22, 23, 24, 25 amino acids to about 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length (e.g., about 105, 110, 115, 120, 130, 140 150 or more amino acids in length). In some embodiments, a peptide linker may be a GS linker.

As used herein, the term “linked,” or “fused” in reference to polynucleotides, refers to the attachment of one polynucleotide to another. In some embodiments, two or more polynucleotide molecules may be linked by a linker that can be an organic molecule, group, polymer, or chemical moiety such as a bivalent organic moiety. A polynucleotide may be linked or fused to another polynucleotide (at the 5′ end or the 3′ end) via a covalent or non-covenant linkage or binding, including e.g., Watson-Crick base-pairing, or through one or more linking nucleotides. In some embodiments, a polynucleotide motif of a certain structure may be inserted within another polynucleotide sequence (e.g., extension of the hairpin structure in the guide RNA). In some embodiments, the linking nucleotides may be naturally occurring nucleotides. In some embodiments, the linking nucleotides may be non-naturally occurring nucleotides.

A “promoter” is a nucleotide sequence that controls or regulates the transcription of a nucleotide sequence (e.g., a coding sequence) that is operably associated with the promoter. The coding sequence controlled or regulated by a promoter may encode a polypeptide and/or a functional RNA. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. A promoter may comprise other elements that act as regulators of gene expression; e.g., a promoter region. These include a TATA box consensus sequence, and often a CAAT box consensus sequence (Breathnach and Chambon, (1981) Annu. Rev. Biochem. 50:349). In plants, the CAAT box may be substituted by the AGGA box (Messing et al., (1983) in Genetic Engineering of Plants, T. Kosuge, C. Meredith and A. Hollaender (eds.), Plenum Press, pp. 211-227).

Promoters useful with this invention can include, for example, constitutive, inducible, temporally regulated, developmentally regulated, chemically regulated, tissue-preferred and/or tissue-specific promoters for use in the preparation of recombinant nucleic acid molecules, e.g., “synthetic nucleic acid constructs” or “protein-RNA complex.” These various types of promoters are known in the art.

The choice of promoter may vary depending on the temporal and spatial requirements for expression, and also may vary based on the host cell to be transformed. Promoters for many different organisms are well known in the art. Based on the extensive knowledge present in the art, the appropriate promoter can be selected for the particular host organism of interest. Thus, for example, much is known about promoters upstream of highly constitutively expressed genes in model organisms and such knowledge can be readily accessed and implemented in other systems as appropriate.

In some embodiments, a promoter functional in a plant may be used with the constructs of this invention. Non-limiting examples of a promoter useful for driving expression in a plant include the promoter of the RubisCo small subunit gene 1 (PrbcS1), the promoter of the actin gene (Pactin), the promoter of the nitrate reductase gene (Pnr) and the promoter of duplicated carbonic anhydrase gene 1 (Pdca1) (See, Walker et al. Plant Cell Rep. 23:727-735 (2005); Li et al. Gene 403:132-142 (2007); Li et al. Mol Biol. Rep. 37:1143-1154 (2010)). PrbcS1 and Pactin are constitutive promoters and Pnr and Pdca1 are inducible promoters. Pnr is induced by nitrate and repressed by ammonium (Li et al. Gene 403:132-142 (2007)) and Pdca1 is induced by salt (Li et al. Mol Biol. Rep. 37:1143-1154 (2010)). In some embodiments, a promoter useful with this invention is RNA polymerase II (Pol II) promoter. In some embodiments, a U6 promoter or a 7SL promoter from Zea mays may be useful with constructs of this invention. In some embodiments, the U6c promoter and/or 7SL promoter from Zea mays may be useful for driving expression of a guide nucleic acid. In some embodiments, a U6c promoter, U6i promoter and/or 7SL promoter from Glycine max may be useful with constructs of this invention. In some embodiments, the U6c promoter, U6i promoter and/or 7SL promoter from Glycine max may be useful for driving expression of a guide nucleic acid.

Examples of constitutive promoters useful for plants include, but are not limited to, cestrum virus promoter (cmp) (U.S. Pat. No. 7,166,770), the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert et al. (1987) Proc. Natl. Acad. Sci USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter. The constitutive promoter derived from ubiquitin accumulates in many cell types. Ubiquitin promoters have been cloned from several plant species for use in transgenic plants, for example, sunflower (Binet et al., 1991. Plant Science 79: 87-94), maize (Christensen et al., 1989. Plant Molec. Biol. 12: 619-632), and arabidopsis (Norris et al. 1993. Plant Molec. Biol. 21:895-906). The maize ubiquitin promoter (UbiP) has been developed in transgenic monocot systems and its sequence and vectors constructed for monocot transformation are disclosed in the patent publication EP 0 342 926. The ubiquitin promoter is suitable for the expression of the nucleotide sequences of the invention in transgenic plants, especially monocotyledons. Further, the promoter expression cassettes described by McElroy et al. (Mol. Gen. Genet. 231: 150-160 (1991)) can be easily modified for the expression of the nucleotide sequences of the invention and are particularly suitable for use in monocotyledonous hosts.

In some embodiments, tissue specific/tissue preferred promoters can be used for expression of a heterologous polynucleotide in a plant cell. Tissue specific or preferred expression patterns include, but are not limited to, green tissue specific or preferred, root specific or preferred, stem specific or preferred, flower specific or preferred or pollen specific or preferred. Promoters suitable for expression in green tissue include many that regulate genes involved in photosynthesis and many of these have been cloned from both monocotyledons and dicotyledons. In one embodiment, a promoter useful with the invention is the maize PEPC promoter from the phosphoenol carboxylase gene (Hudspeth & Grula, Plant Molec. Biol. 12:579-589 (1989)). Non-limiting examples of tissue-specific promoters include those associated with genes encoding the seed storage proteins (such as β-conglycinin, cruciferin, napin and phaseolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. (1991) Seed Sci. Res. 1:209-219; as well as EP Patent No. 255378). Tissue-specific or tissue-preferential promoters useful for the expression of the nucleotide sequences of the invention in plants, particularly maize, include but are not limited to those that direct expression in root, pith, leaf or pollen. Such promoters are disclosed, for example, in WO93/07278, herein incorporated by reference in its entirety. Other non-limiting examples of tissue specific or tissue preferred promoters useful with the invention the cotton rubisco promoter disclosed in U.S. Pat. 6,040,504; the rice sucrose synthase promoter disclosed in U.S. Pat. 5,604,121; the root specific promoter described by de Framond (FEBS 290:103-106 (1991); EP 0 452 269 to Ciba- Geigy); the stem specific promoter described in U.S. Pat. 5,625,136 (to Ciba-Geigy) and which drives expression of the maize trpA gene; the cestrum yellow leaf curling virus promoter disclosed in WO 01/73087; and pollen specific or preferred promoters including, but not limited to, ProOsLPS10 and ProOsLPS11 from rice (Nguyen et al. Plant Biotechnol. Reports 9(5):297-306 (2015)), ZmSTK2_USP from maize (Wang et al. Genome 60(6):485-495 (2017)), LAT52 and LAT59 from tomato (Twell et al. Development 109(3):705-713 (1990)), Zm13 (U.S. Pat. No. 10,421,972), PLA₂-δ promoter from arabidopsis (U.S. Pat. No. 7,141,424), and/or the ZmC5 promoter from maize (International PCT Publication No. WO1999/042587.

Additional examples of plant tissue-specific/tissue preferred promoters include, but are not limited to, the root hair-specific cis-elements (RHEs) (Kim et al. The Plant Cell 18:2958-2970 (2006)), the root-specific promoters RCc3 (Jeong et al. Plant Physiol. 153:185-197 (2010)) and RB7 (U.S. Pat. No. 5459252), the lectin promoter (Lindstrom et al. (1990) Der. Genet. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), S-adenosyl-L-methionine synthetase (SAMS) (Vander Mijnsbrugge et al. (1996) Plant and Cell Physiology, 37(8):1108-1115), corn light harvesting complex promoter (Bansal et al. (1992) Proc. Natl. Acad. Sci. USA 89:3654-3658), corn heat shock protein promoter (O′Dell et al. (1985) EMBO J. 5:451-458; and Rochester et al. (1986) EMBO J. 5:451-458), pea small subunit RuBP carboxylase promoter (Cashmore, “Nuclear genes encoding the small subunit of ribulose-1,5-bisphosphate carboxylase” pp. 29-39 In: Genetic Engineering of Plants (Hollaender ed., Plenum Press 1983; and Poulsen et al. (1986) Mol. Gen. Genet. 205:193-200), Ti plasmid mannopine synthase promoter (Langridge et al. (1989) Proc. Natl. Acad. Sci. USA 86:3219-3223), Ti plasmid nopaline synthase promoter (Langridge et al. (1989), supra), petunia chalcone isomerase promoter (van Tunen et al. (1988) EMBO J. 7:1257-1263), bean glycine rich protein 1 promoter (Keller et al. (1989) Genes Dev. 3:1639-1646), truncated CaMV 35S promoter (O′Dell et al. (1985) Nature 313:810-812), potato patatin promoter (Wenzler et al. (1989) Plant Mol. Biol. 13:347-354), root cell promoter (Yamamoto et al. (1990) Nucleic Acids Res. 18:7449), maize zein promoter (Kriz et al. (1987) Mol. Gen. Genet. 207:90-98; Langridge et al. (1983) Cell 34:1015-1022; Reina et al. (1990) Nucleic Acids Res. 18:6425; Reina et al. (1990) Nucleic Acids Res. 18:7449; and Wandelt et al. (1989) Nucleic Acids Res. 17:2354), globulin-1 promoter (Belanger et al. (1991) Genetics 129:863-872), α-tubulin cab promoter (Sullivan et al. (1989) Mol. Gen. Genet. 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBO J. 10:2605-2612).

Useful for seed-specific expression is the pea vicilin promoter (Czako et al. (1992) Mol. Gen. Genet. 235:33-40; as well as the seed-specific promoters disclosed in U.S. Pat. No. 5,625,136. Useful promoters for expression in mature leaves are those that are switched at the onset of senescence, such as the SAG promoter from Arabidopsis (Gan et al. (1995) Science 270:1986-1988).

In addition, promoters functional in chloroplasts can be used. Non-limiting examples of such promoters include the bacteriophage T3 gene 9 5′ UTR and other promoters disclosed in U.S. Pat. No. 7,579,516. Other promoters useful with the invention include but are not limited to the S-E9 small subunit RuBP carboxylase promoter and the Kunitz trypsin inhibitor gene promoter (Kti3).

Additional regulatory elements useful with this invention include, but are not limited to, introns, enhancers, termination sequences and/or 5′ and 3′ untranslated regions.

An intron useful with this invention can be an intron identified in and isolated from a plant and then inserted into an expression cassette to be used in transformation of a plant. As would be understood by those of skill in the art, introns can comprise the sequences required for self-excision and are incorporated into nucleic acid constructs/expression cassettes in frame. An intron can be used either as a spacer to separate multiple protein-coding sequences in one nucleic acid construct, or an intron can be used inside one protein-coding sequence to, for example, stabilize the mRNA. If they are used within a protein-coding sequence, they are inserted “in-frame” with the excision sites included. Introns may also be associated with promoters to improve or modify expression. As an example, a promoter/intron combination useful with this invention includes but is not limited to that of the maize Ubi1 promoter and intron (see, e.g., SEQ ID NO:21 and SEQ ID NO:22).

Non-limiting examples of introns useful with the present invention include introns from the ADHI gene (e.g., Adh1-S introns 1, 2 and 6), the ubiquitin gene (Ubi1), the RuBisCO small subunit (rbcS) gene, the RuBisCO large subunit (rbcL) gene, the actin gene (e.g., actin-1 intron), the pyruvate dehydrogenase kinase gene (pdk), the nitrate reductase gene (nr), the duplicated carbonic anhydrase gene 1 (Tdca1), the psbA gene, the αtpA gene, or any combination thereof.

In some embodiments, a polynucleotide and/or a nucleic acid construct of the invention can be an “expression cassette” or can be comprised within an expression cassette. As used herein, “expression cassette” means a recombinant nucleic acid molecule comprising, for example, a one or more polynucleotides of the invention (e.g., a polynucleotide encoding a sequence-specific nucleic acid binding domain, a polynucleotide encoding a deaminase protein or domain, a polynucleotide encoding a reverse transcriptase protein or domain, a polynucleotide encoding a 5′-3′ exonuclease polypeptide or domain, a guide nucleic acid and/or reverse transcriptase (RT) template), wherein polynucleotide(s) is/are operably associated with one or more control sequences (e.g., a promoter, terminator and the like). Thus, in some embodiments, one or more expression cassettes may be provided, which are designed to express, for example, a nucleic acid construct of the invention (e.g., a polynucleotide encoding a sequence-specific nucleic acid binding domain, a polynucleotide encoding a nuclease polypeptide/domain, a polynucleotide encoding a deaminase protein/domain, a polynucleotide encoding a reverse transcriptase protein/domain, a polynucleotide encoding a 5′-3′ exonuclease polypeptide/domain, a polynucleotide encoding a peptide tag, and/or a polynucleotide encoding an affinity polypeptide, and the like, or comprising a guide nucleic acid, an extended guide nucleic acid, and/or RT template, and the like). When an expression cassette of the present invention comprises more than one polynucleotide, the polynucleotides may be operably linked to a single promoter that drives expression of all of the polynucleotides or the polynucleotides may be operably linked to one or more separate promoters (e.g., three polynucleotides may be driven by one, two or three promoters in any combination). When two or more separate promoters are used, the promoters may be the same promoter, or they may be different promoters. Thus, a polynucleotide encoding a sequence specific nucleic acid binding domain, a polynucleotide encoding a nuclease protein/domain, a polynucleotide encoding a CRISPR-Cas effector protein/domain, a polynucleotide encoding an deaminase protein/domain, a polynucleotide encoding a reverse transcriptase polypeptide/domain (e.g., RNA-dependent DNA polymerase), and/or a polynucleotide encoding a 5′-3′ exonuclease polypeptide/domain, a guide nucleic acid, an extended guide nucleic acid and/or RT template when comprised in a single expression cassette may each be operably linked to a single promoter, or separate promoters in any combination.

An expression cassette comprising a nucleic acid construct of the invention may be chimeric, meaning that at least one (e.g., one or more) of its components is heterologous with respect to at least one of its other components (e.g., a promoter from the host organism operably linked to a polynucleotide of interest to be expressed in the host organism, wherein the polynucleotide of interest is from a different organism than the host or is not normally found in association with that promoter). An expression cassette may also be one that is naturally occurring but has been obtained in a recombinant form useful for heterologous expression.

An expression cassette can optionally include a transcriptional and/or translational termination region (i.e., termination region) and/or an enhancer region that is functional in the selected host cell. A variety of transcriptional terminators and enhancers are known in the art and are available for use in expression cassettes. Transcriptional terminators are responsible for the termination of transcription and correct mRNA polyadenylation. A termination region and/or the enhancer region may be native to the transcriptional initiation region, may be native to, for example, a gene encoding a sequence-specific nucleic acid binding protein, a gene encoding a nuclease, a gene encoding a reverse transcriptase, a gene encoding a deaminase, and the like, or may be native to a host cell, or may be native to another source (e.g., foreign or heterologous to, for example, to a promoter, to a gene encoding a sequence-specific nucleic acid binding protein, a gene encoding a nuclease, a gene encoding a reverse transcriptase, a gene encoding a deaminase, and the like, or to the host cell, or any combination thereof).

An expression cassette of the invention also can include a polynucleotide encoding a selectable marker, which can be used to select a transformed host cell. As used herein, “selectable marker” means a polynucleotide sequence that when expressed imparts a distinct phenotype to the host cell expressing the marker and thus allows such transformed cells to be distinguished from those that do not have the marker. Such a polynucleotide sequence may encode either a selectable or screenable marker, depending on whether the marker confers a trait that can be selected for by chemical means, such as by using a selective agent (e.g., an antibiotic and the like), or on whether the marker is simply a trait that one can identify through observation or testing, such as by screening (e.g., fluorescence). Many examples of suitable selectable markers are known in the art and can be used in the expression cassettes described herein.

In addition to expression cassettes, the nucleic acid molecules/constructs and polynucleotide sequences described herein can be used in connection with vectors. The term “vector” refers to a composition for transferring, delivering or introducing a nucleic acid (or nucleic acids) into a cell. A vector comprises a nucleic acid construct (e.g., expression cassette(s)) comprising the nucleotide sequence(s) to be transferred, delivered or introduced. Vectors for use in transformation of host organisms are well known in the art. Non-limiting examples of general classes of vectors include viral vectors, plasmid vectors, phage vectors, phagemid vectors, cosmid vectors, fosmid vectors, bacteriophages, artificial chromosomes, minicircles, or Agrobacterium binary vectors in double or single stranded linear or circular form which may or may not be self-transmissible or mobilizable. In some embodiments, a viral vector can include, but is not limited, to a retroviral, lentiviral, adenoviral, adeno-associated, or herpes simplex viral vector. A vector as defined herein can transform a prokaryotic or eukaryotic host either by integration into the cellular genome or exist extrachromosomally (e.g., autonomous replicating plasmid with an origin of replication). Additionally, included are shuttle vectors by which is meant a DNA vehicle capable, naturally or by design, of replication in two different host organisms, which may be selected from actinomycetes and related species, bacteria and eukaryotic (e.g., higher plant, mammalian, yeast or fungal cells). In some embodiments, the nucleic acid in the vector is under the control of, and operably linked to, an appropriate promoter or other regulatory elements for transcription in a host cell. The vector may be a bi-functional expression vector which functions in multiple hosts. In the case of genomic DNA, this may contain its own promoter and/or other regulatory elements and in the case of cDNA this may be under the control of an appropriate promoter and/or other regulatory elements for expression in the host cell. Accordingly, a nucleic acid or polynucleotide of this invention and/or expression cassettes comprising the same may be comprised in vectors as described herein and as known in the art.

As used herein, “contact,” “contacting,” “contacted,” and grammatical variations thereof, refer to placing the components of a desired reaction together under conditions suitable for carrying out the desired reaction (e.g., transformation, transcriptional control, genome editing, nicking, and/or cleavage). As an example, a target nucleic acid may be contacted with a sequence-specific nucleic acid binding protein (e.g., polynucleotide-guided endonuclease, a CRISPR-Cas endonuclease (e.g., CRISPR-Cas effector protein), a zinc finger nuclease, a transcription activator-like effector nuclease (TALEN) and/or an Argonaute protein)) and a deaminase or a nucleic acid construct encoding the same, under conditions whereby the sequence-specific nucleic acid binding protein, the reverse transcriptase and/or the deaminase are expressed and the sequence-specific nucleic acid binding protein binds to the target nucleic acid, and the reverse transcriptase and/or deaminase may be fused to either the sequence-specific nucleic acid binding protein or recruited to the sequence-specific nucleic acid binding protein (via, for example, a peptide tag fused to the sequence-specific nucleic acid binding protein and an affinity tag fused to the reverse transcriptase and/or deaminase) and thus, the deaminase and/or reverse transcriptase is positioned in the vicinity of the target nucleic acid, thereby modifying the target nucleic acid. Other methods for recruiting reverse transcriptase and/or deaminase may be used that take advantage of other protein-protein interactions, and also RNA-protein interactions and chemical interactions may be used for protein-protein and protein-nucleic acid recruitment.

As used herein, “modifying” or “modification” in reference to a target nucleic acid includes editing (e.g., mutating), covalent modification, exchanging/substituting nucleic acids/nucleotide bases, deleting, cleaving, nicking, and/or altering transcriptional control of a target nucleic acid. In some embodiments, a modification may include one or more single base changes (SNPs) of any type.

“Introducing,” “introduce,” “introduced” (and grammatical variations thereof) in the context of a polynucleotide of interest means presenting a nucleotide sequence of interest (e.g., polynucleotide, RT template, a nucleic acid construct, and/or a guide nucleic acid) to a plant, plant part thereof, or cell thereof, in such a manner that the nucleotide sequence gains access to the interior of a cell.

The terms “transformation” or “transfection” may be used interchangeably and as used herein refer to the introduction of a heterologous nucleic acid into a cell. Transformation of a cell may be stable or transient. Thus, in some embodiments, a host cell or host organism (e.g., a plant) may be stably transformed with a polynucleotide/nucleic acid molecule of the invention. In some embodiments, a host cell or host organism may be transiently transformed with a polynucleotide/nucleic acid molecule of the invention.

“Transient transformation” in the context of a polynucleotide means that a polynucleotide is introduced into the cell and does not integrate into the genome of the cell.

By “stably introducing” or “stably introduced” in the context of a polynucleotide introduced into a cell is intended that the introduced polynucleotide is stably incorporated into the genome of the cell, and thus the cell is stably transformed with the polynucleotide.

“Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein includes the nuclear and the plastid genome, and therefore includes integration of the nucleic acid into, for example, the chloroplast or mitochondrial genome. Stable transformation as used herein can also refer to a transgene that is maintained extrachromasomally, for example, as a minichromosome or a plasmid.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant). Stable transformation of a cell can be detected by, for example, a Northern blot hybridization assay of RNA of the cell with nucleic acid sequences which specifically hybridize with a nucleotide sequence of a transgene introduced into a host organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods. Transformation can also be detected by direct sequencing and/or hybridization protocols well known in the art.

Accordingly, in some embodiments, nucleotide sequences, polynucleotides, nucleic acid constructs, and/or expression cassettes of the invention may be expressed transiently and/or they can be stably incorporated into the genome of the host organism. Thus, in some embodiments, a nucleic acid construct of the invention (e.g., one or more expression cassettes comprising polynucleotides for editing as described herein) may be transiently introduced into a cell with a guide nucleic acid and as such, no DNA is maintained in the cell.

A nucleic acid construct of the invention may be introduced into a plant cell by any method known to those of skill in the art. Non-limiting examples of transformation methods include transformation via bacterial-mediated nucleic acid delivery (e.g., via Agrobacteria), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. Procedures for transforming both eukaryotic and prokaryotic organisms are well known and routine in the art and are described throughout the literature (See, for example, Jiang et al. 2013. Nat. Biotechnol. 31:233-239; Ran et al. Nature Protocols 8:2281-2308 (2013)). General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

In some embodiments of the invention, transformation of a cell may comprise nuclear transformation. In other embodiments, transformation of a cell may comprise plastid transformation (e.g., chloroplast transformation). In still further embodiments, nucleic acids of the invention may be introduced into a cell via conventional breeding techniques. In some embodiments, one or more of the polynucleotides, expression cassettes and/or vectors may be introduced into a plant cell via Agrobacterium transformation.

A polynucleotide therefore can be introduced into a plant, plant part, plant cell in any number of ways that are well known in the art. The methods of the invention do not depend on a particular method for introducing one or more nucleotide sequences into a plant, only that they gain access to the interior the cell. Where more than polynucleotide is to be introduced, they can be assembled as part of a single nucleic acid construct, or as separate nucleic acid constructs, and can be located on the same or different nucleic acid constructs. Accordingly, the polynucleotide can be introduced into the cell of interest in a single transformation event, or in separate transformation events, or, alternatively, a polynucleotide can be incorporated into a plant as part of a breeding protocol.

The present invention is directed to modification of Ribosomal Protein L3 (RPL3) genes (e.g., RPL3A gene, a RPL3A-1 gene, a RPL3A-2 gene, a RPL3A-3 gene, a RPL3B gene, a RPL3B-1 gene, a RPL3B-2 gene, and/or a RPL3B-3 gene) in plants through editing technology to provide plants that exhibit reduced binding of a trichothecene mycotoxin to the RPL3 polypeptide encoded by the modified RPL3 gene, that have an increased tolerance to deoxynivalenol (DON), that have increased resistance to fusarium head blight (FHB) (e.g., increased resistance to the FHB causal agent, Fusarium graminearum, e.g., decreased symptoms FHB infection, e.g., decreased symptoms of infection by Fusarium graminearum)), and/or have a reduced Fusarium graminearum load. Specifically, RPL3 genes encode Ribosomal Protein L3 (RPL3) polypeptides, which interact with the Fusarium head blight (FHB) virulence factors, trichothecene mycotoxins. In particular, the trichothecene mycotoxin, DON, is believed to act as a virulence factor by inhibiting eukaryotic protein synthesis by binding to Ribosomal Protein L3 (RPL3). Thus, without wishing to be limited by any particular theory, the ability of the fungus to infect the plant or part thereof may be reduced by modifying the RPL3 gene such that DON has a reduced ability or no ability to bind the encoded RPL3 polypeptide, thereby resulting in a plant or part thereof having increased resistance/tolerance to infection by the fungus, decreased symptoms of FHB infection, a reduced fungal load, and/or a reduced accumulation of mycotoxins in infected plant parts, such as wherein the plant may produce seed that accumulates less trichothecene mycotoxin (e.g., DON) when infected with Fusarium graminearum. The modifications in an endogenous RPL3 gene encoding an RPL3 polypeptide described herein may regulate the disease response to Fusarium graminearum, may provide increased tolerance to mycotoxins produced by F. graminearum, may result in the plant or part thereof comprising a reduced F. graminearum load and/or may result in a plant or part thereof containing a reduced amount of mycotoxin (e.g., a seed having a reduced level of mycotoxin as compared to a seed from a plant not comprising a mutation in at least one endogenous RPL3 gene as described herein.

In wheat, RPL3 genes include RPL3A, RPL3A-1, RPL3A-2, RPL3A-3, RPL3B, RPL3B-1, RPL3B-2, and/or RPL3B-3, each of which, or any combination thereof, may be targeted in a plant. Thus, an editing strategy useful for this invention can include generating a mutation in one or more than one RPL3 gene (e.g., 1, 2, 3, 4, 5, and/or 6 RPL3 genes). As an example, one or more mutations may be generated in one or more of a RPL3A gene and/or one or more of a RPL3B gene of a plant. In some embodiments, one or more mutations may be generated in one or more (1, 2, or all 3) of a RPL3A-1 gene, a RPL3A-2 gene, and/or a RPL3A-3 gene, and/or one or more of a RPL3B-1 gene, RPL3B-2 gene, and/or RPL3B-3. In some embodiments, a mutation may be a non-natural mutation. Mutations that may be useful for producing plants exhibiting (1) reduced binding of a trichothecene mycotoxin to the RPL3 polypeptide encoded by a modified RPL3 gene, (2) increased tolerance to deoxynivalenol (DON), (3) increased resistance to fusarium head blight (FHB) (e.g., increased resistance to the FHB causal agent, Fusarium graminearum, e.g., decreased symptoms FHB infection, e.g., decreased symptoms of infection by Fusarium graminearum), and/or (4) reduced Fusarium graminearum load, include, for example, substitutions, deletions, and/or insertions. In some aspects, a mutation generated by the editing technology can be a point mutation. In some embodiments, a mutation in one or more than one RPL3 gene as described herein results in knockdown of expression of the one or more than one RPL3 gene, e.g., reduce production of the encoded polypeptide. In some embodiments, a mutation in one or more than one RPL3 gene as described herein results in a knockout of the one or more than one RPL3 gene, resulting in no detectable production of the encoded polypeptide. In some embodiments, a mutated RPL3 gene is provided that comprises a nucleic acid sequence having at least 90% sequence identity to any one of SEQ ID NOs:147, 149, 151, 153, or 155 and/or that encodes a mutated RPL3 polypeptide having at least 90% sequence identity to any one of SEQ ID NOs:148, 150, 152, 154, or 156.

In some embodiments, the invention provides a plant or part thereof comprising at least one mutation in an endogenous Ribosomal Protein L3 (RPL3) gene (e.g., in one or more than one RPL3 gene) that encodes a RPL3 polypeptide, optionally, wherein the at least one mutation of the endogenous RPL3 gene results in no detectable RPL3 polypeptide or a RPL3 polypeptide having reduced binding to a trichothecene mycotoxin. Without being limited by any particular theory, the RPL3 polypeptide is believed to be capable of regulating disease response to Fusarium graminearum and/or increased tolerance to deoxynivalenol (DON).

In some embodiments, an endogenous RPL3 gene is an endogenous RPL3A gene or an endogenous RPL3B gene, wherein the encoded RPL3 polypeptide is a RPL3A polypeptide, or a RPL3B polypeptide, respectively. In some embodiments, a mutation in in one or more than one RPL3 gene may be non-natural mutations. In some embodiments, a mutation in an RPL3 gene may be a null mutation. In some embodiments, a mutation may be a knock-out mutation or a knock-down mutation. As used herein, a knock-out mutation may result in little or no expression and/or an encoded RPL3 polypeptide having no (zero percent) activity. A knock-down mutation may result in the decreased expression of a modified RPL3 polypeptide, which RPL3 polypeptide when produced may have a reduced ability to bind a trichothecene mycotoxin (e.g., DON) by at least 5% (e.g., a reduction in binding of a mycotoxin by a mutated RPL3 polypeptide (produced by the mutated endogenous RPL3 gene) of about 5, 6, 7, 8, 9, 10, 11, 12, 13, 14, 15, 16, 17, 18, 19, 20, 21, 22, 23, 24, 25, 26, 27, 28, 29, 30, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, and any range or value therein).

In some embodiments, the plant or part thereof is a monocot. In some embodiments, the plant is a wheat plant, a barley plant, a spelt plant, or an oat plant. In some embodiments, the plant is a wheat plant, and a mutation in an endogenous RPL3 gene may be in the A genome, optionally in chromosome 4A or in chromosome 5A, in the B genome, in the D genome, or in any combination thereof, optionally in the A genome and the B genome, optionally wherein the endogenous RPL3 gene is a RPL3A gene and/or a RPL3B gene. In some embodiments the RPL3A gene is a RPL3A-1 gene, a RPL3A-2 gene, and/or a RPL3A-3 gene and/or the RPL3B gene is a RPL3B-1 gene, a RPL3B-2 gene, and/or a RPL3B-3 gene.

FHB resistance is a quantitative trait, and it has been classically proposed and well-accepted that wheat can present five different types of resistance to this disease: (I) resistance to the initial infection of the pathogen within spike tissues; (II) resistance to the subsequent spread of the disease throughout the spike; (III) resistance to the accumulation of mycotoxins; (IV) resistance to kernel infection and/or (V) resistance to yield performance (Venske et al. Front. Plant Sci. 13 Jun. 2019; doi.org/10.3389/fpls.2019.00727)). Thus, in addition to a mutation in an endogenous RPL3 gene as described herein, a plant or part thereof the invention may further comprise one or more additional Fusarium head blight resistance alleles. In some embodiments, the one or more additional Fusarium head blight resistance alleles may be one or more quantitative trait loci (QTL). Venske et al. (Front. Plant Sci. 13 Jun. 2019; doi.org/10.3389/fpls.2019.00727) reported that a total of 556 QTL are distributed on all sub-genomes and chromosomes of wheat. In some embodiments, a plant of this invention comprising a mutation in an RPL3 gene as described herein may further comprise one or more (e.g., 1, 2, 3, 4, 5, 6, 7, 8, 9, 10, 11, 12, 13, 14, 15, 16, 17, 18, 19, or 20 or more) QTLs associated with Fusarium head blight resistance (e.g., stacked), wherein the QTL can include, but is not limited to, Fhb1, Fhb2, Fhb3, Fhb4, Fhb5, Fhb6, and/or Fhb7.

In some embodiments, a plant cell is provided, the plant cell comprising an editing system, the editing system comprising: (a) a CRISPR-Cas effector protein; and (b) a guide nucleic acid (e.g., gRNA, gDNA, crRNA, crDNA, sgRNA, sgDNA) comprising a spacer sequence with complementarity to an endogenous target gene encoding a Ribosomal Protein L3 (RPL3) polypeptide. The editing system may be used to generate a mutation in the endogenous target gene encoding a RPL3 polypeptide. In some embodiments, the endogenous target gene is an endogenous Ribosomal Protein L3 (RPL3) gene (e.g., one or more than one endogenous RPL3 gene), optionally an endogenous RPL3A gene and/or an endogenous RPL3B gene, optionally an endogenous RPL3A-1 gene, RPL3A-2 gene, RPL3A-3 gene, RPL3B-1 gene, RPL3B-2 gene, and/or RPL3B-3 gene. In some embodiments, the mutation may be a non-natural mutation. In some embodiments, the endogenous target gene: (a) comprises a nucleotide sequence having at least 80% sequence identity to any one of SEQ ID NO:72, 73, 75, 76, 78, 79, 80, 81, 83, 84, 86, or 87, (b) comprises a region having at least 80% sequence identity to any one of SEQ ID NOs:89-139, (c) encodes a sequence having at least 80% sequence identity to any one of SEQ ID NO:74, 77, 82, 85, or 88. In some embodiments, a guide nucleic acid of an editing system may comprise the nucleotide sequence (a spacer sequence, e.g., one or more spacers, or reverse complements thereof) of any one of SEQ ID NO:140-146.

A mutation in a RPL3 gene of the plant, plant part thereof, or the plant cell useful for this invention may be any type of mutation, including a base substitution, a base deletion, and/or a base insertion. In some embodiments, a non-natural mutation may comprise a base substitution to an A, a T, a G, or a C. In some embodiments, a mutation may be a deletion of at least one base pair (e.g., 1 base pair to about 100 base pairs; e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 consecutive base pairs; e.g., 1 to about 50 consecutive base pairs, 1 to about 30 consecutive base pairs, 1 to about 15 consecutive base pairs) or an insertion of at least one base pair (e.g., 1 base pair to about 16 base pairs; e.g., 1, 2, 3, 4, 5, 6, 7, 8, 9, 10, 11, 12, 13, 14, 15 or 16 consecutive base pairs), optionally wherein the deletion or insertion is an out-of-frame deletion or an out-of-frame insertion. In some embodiments, a mutation in one or more than one RPL3 gene may be a non-natural mutation. In some embodiments, a mutated endogenous RPL3 gene comprises a nucleic acid sequence having at least 90% sequence identity to any one of SEQ ID NOs:147, 149, 151, 153, or 155 and/or encodes a mutated RPL3 polypeptide having at least 90% sequence identity to any one of SEQ ID NOs:148, 150, 152, 154, or 156.

A mutation in a RPL3 gene may be located in the 5′ region of the RPL3 gene (e.g., a RPL3A gene and/or a RPL3B gene), optionally in the 5′ region of the RPL3 gene that encodes the N-terminal region of the encoded RPL3 polypeptide (e.g., the 5′ coding regions (exons)). In some embodiments, the mutation may be an out-of-frame deletion or an out-of-frame insertion that may result in a truncated polypeptide, or little or no detectible polypeptide. In some embodiments, the out-of-frame deletion or out-of-frame insertion may be a null mutation. In some embodiments, a mutation located in the 5′ region of the RPL3 gene may be a deletion or an insertion that results in a premature stop codon (e.g., an out-of-frame base insertion or an out-of-frame base deletion) and a truncated RPL3 polypeptide or, optionally resulting in little or no detectable RPL3 polypeptide. In some embodiments, the mutation may result in a truncated RPL3 polypeptide, optionally a C-terminal truncation of the RPL3 polypeptide, optionally wherein the C-terminal truncation results in a deletion of about 50 amino acid residues to about 300 amino acid residues from the C-terminal portion of the polypeptide (e.g., about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 1699, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 255, 260, 270, 275, 280, 285, 290, 295, or 300 or more residues, or any range or value therein, from the C-terminus of the RPL3 polypeptide, e.g., SEQ ID NO:74, 77, 82, 85, or 88).

The types of editing tools that may be used to generate these and other mutations in RPL3 genes include any base editors or cutters, which are guided to a target site using spacers having at least 80% complementarity to a portion or a region of a RPL3 gene (e.g., one or more than one RPL3 gene, e.g., a RPL3A gene and/or a RPL3B gene) as described herein.

In some embodiments, a mutation of a RPL3 gene is within a portion or region of the endogenous RPL3 gene, the portion or region having at least 80% (80, 81, 82, 83, 84, 85, 86, 87, 88, 89, 90, 91, 92, 92, 94, 95, 96, 97, 98, 99, or 100%) sequence identity to any one of the nucleotide sequences of SEQ ID NOs:89-139.

An endogenous RPL3 gene useful with this invention (e.g., an endogenous target gene) encodes a Ribosomal Protein L3 (RPL3) polypeptide, and includes an endogenous RPL3A gene, an endogenous RPL3B gene, an endogenous RPL3A-1 gene, an endogenous RPL3A-2 gene, an endogenous RPL3A-3 gene, an endogenous RPL3B-1 gene, an endogenous RPL3B-2 gene, and/or an endogenous RPL3B-3 gene. In some embodiments, an endogenous RPL3 gene (e.g., endogenous target gene) (1) may comprise a nucleic acid sequence having at least 80% sequence identity to any one of SEQ ID NO:72, 73, 75, 76, 78, 79, 80, 81, 83, 84, 86, or 87, (2) may comprise a region of a RPL3 gene having at least 80% sequence identity to any one of SEQ ID NOs:89-139, and/or (3) may encode a RPL3 polypeptide having at least 80% sequence identity to any one of SEQ ID NO:74, 77, 82, 85, or 88.

In some embodiments, a plant (for example, a wheat plant, a spelt plant, an oat plant or an oat plant) comprising at least one (e.g., one or more) mutation, optionally a non-natural mutation, in an endogenous RPL3 gene (in at least one endogenous RPL3 gene, e.g., in one or more RPL3 genes) exhibits (1) reduced binding of a trichothecene mycotoxin to the RPL3 polypeptide encoded by a modified RPL3 gene, (2) increased tolerance to deoxynivalenol (DON), (3) increased resistance/tolerance to fusarium head blight (FHB) (e.g., increased resistance to the FHB causal agent, Fusarium graminearum, and/or reduced symptoms of FHB), (4) reduced Fusarium graminearum load and/or (5) produces seed that accumulates less trichothecene mycotoxin (e.g., DON) when infected with Fusarium graminearum as compared to a plant devoid of the at least one mutation (e.g., an isogenic plant (e.g., wild type unedited plant or a null segregant). In some embodiments, a plant may comprise a mutated endogenous RPL3 gene comprising a nucleic acid sequence having at least 90% sequence identity to any one of SEQ ID NOs:147, 149, 151, 153, or 155 and/or encoding a mutated RPL3 polypeptide having at least 90% sequence identity to any one of SEQ ID NOs:148, 150, 152, 154, or 156.

In some embodiments, a plant may be regenerated from a plant part and/or plant cell of the invention comprising a mutation in one or more than one endogenous RPL3 gene (an endogenous RPL3A gene and/or an endogenous RPL3B gene) as described herein, wherein the regenerated plant comprises the mutation in the one or more than one endogenous RPL3 gene and a phenotype of (1) reduced binding of a trichothecene mycotoxin to the RPL3 polypeptide encoded by a modified endogenous RPL3 gene, (2) increased tolerance to deoxynivalenol (DON), (3) increased resistance/tolerance to fusarium head blight (FHB) (e.g., increased resistance to the FHB causal agent, Fusarium graminearum and/or reduced symptoms of FHB), (4) reduced Fusarium graminearum load, and/or (5) seed that accumulates less trichothecene mycotoxin (e.g., DON) when infected with Fusarium graminearum as compared to a plant devoid of the at least one mutation (e.g., an isogenic plant (e.g., wild type unedited plant or a null segregant). In some embodiments, a regenerated plant may comprise a mutated endogenous RPL3 gene having at least 90% sequence identity to any one of SEQ ID NOs:147, 149, 151, 153, or 155 and/or that encodes a mutated RPL3 polypeptide having at least 90% sequence identity to any one of SEQ ID NOs:148, 150, 152, 154, or 156.

In some embodiments, a plant cell is provided, the plant cell comprising at least one (e.g., one or more) mutation within an endogenous Ribosomal Protein L3 (RPL3) gene, wherein the at least one mutation is a substitution, insertion, or deletion that is introduced using an editing system that comprises a nucleic acid binding domain that binds to a target site in the endogenous RPL3 gene. In some embodiments, the mutation may be a non-natural mutation. In some embodiments, the substitution, insertion, or deletion results in, for example, an amino acid substitution. In some embodiments, the substitution, insertion, or deletion results in, for example, a premature stop codon. In some embodiments, the substitution, insertion, or deletion results in, for example, a truncated RPL3 protein and/or the absence of the RPL3 protein (e.g., the truncation results in no or little detectable protein). In some embodiments, the at least one mutation may be a point mutation, optionally resulting in a premature stop codon, optionally a truncated RPL3 protein, optionally no or little or no detectable RPL3 protein. In some embodiments, the at least one mutation within the RPL3 gene is an insertion and/or a deletion, optionally the at least one mutation may be an out-of-frame insertion or out-of-frame deletion. In some embodiments, the endogenous RPL3 gene is an endogenous RPL3A gene and/or an endogenous RPL3B gene, optionally wherein the endogenous RPL3 gene may be an endogenous RPL3A-1 gene, an endogenous RPL3A-2 gene, an endogenous RPL3A-3 gene, an endogenous RPL3B-1 gene, an endogenous RPL3B-2 gene, and/or an endogenous RPL3B-3 gene. In some embodiments, the plant cell may comprise a mutated endogenous RPL3 gene having at least 90% sequence identity to any one of SEQ ID NOs:147, 149, 151, 153, or 155 and/or that encodes a mutated RPL3 polypeptide having at least 90% sequence identity to any one of SEQ ID NOs:148, 150, 152, 154, or 156.

In some embodiments, a target site in a RPL3 gene of a plant cell may be within a region or portion of the endogenous RPL3 gene, the region having at least 80% sequence identity to any one of the nucleotide sequences of SEQ ID NOs:89-139.

In some embodiments, a mutation may be made following cleavage by an editing system that comprises a nuclease and a nucleic acid binding domain that binds to a target site within a sequence having least 80% sequence identity to a sequence encoding of any one of SEQ ID NO:72, 73, 75, 76, 78, 79, 80, 81, 83, 84, 86, or 87, optionally within the 5′ region of a sequence having least 80% sequence identity to a sequence encoding of any one of SEQ ID NO:72, 73, 75, 76, 78, 79, 80, 81, 83, 84, 86, or 87, optionally in or adjacent to the third exon of a RPL3 genomic sequence, or a sequence having at least 80% sequence identity to a sequence encoding any one of SEQ ID NOs:89-139, and the at least one mutation within a RPL3 gene is made following cleavage by the nuclease. In some embodiments, the at least one mutation may result in a null allele.

In some embodiments, a plant cell may be regenerated into a plant that comprises the at least one mutation, optionally a non-natural mutation, optionally wherein the plant regenerated from the plant cell exhibits a phenotype of (1) reduced binding of a trichothecene mycotoxin to the RPL3 polypeptide encoded by a modified endogenous RPL3 gene, (2) increased tolerance to deoxynivalenol (DON), (3) increased resistance/tolerance to fusarium head blight (FHB) (e.g., increased resistance to the FHB causal agent, Fusarium graminearum and/or reduced symptoms of FHB), (4) reduced Fusarium graminearum load, and/or (5) producing seed that accumulates less trichothecene mycotoxin (e.g., DON) when infected with Fusarium graminearum as compared to a plant devoid of the at least one mutation (e.g., an isogenic plant (e.g., wild type unedited plant or a null segregant).

In some embodiments, a method of producing/breeding a transgene-free edited plant (e.g., a wheat plant, a spelt plant, an oat plant, a barely plant) is provided, the method comprising: crossing a plant of the present invention (e.g., a plant comprising one or more mutations (e.g., non-natural mutations) in one or more RPL3 genes and having a phenotype of (1) reduced binding of a trichothecene mycotoxin to the RPL3 polypeptide encoded by a modified RPL3 gene, (2) increased tolerance to deoxynivalenol (DON), (3) increased resistance/tolerance to fusarium head blight (FHB) (e.g., increased resistance to the FHB causal agent, Fusarium graminearum and/or reduced symptoms of FHB), (4) reduced Fusarium graminearum load, and/or (5) producing seed that accumulates less trichothecene mycotoxin (e.g., DON) when infected with Fusarium graminearum with a transgene free plant, thereby introducing the mutation into the plant that is transgene-free; and selecting a progeny plant that comprises the mutation and is transgene-free, thereby producing a transgene free edited plant.

Also provided herein is a method of providing a plurality of plants (e.g., wheat plants, spelt plants, oat plants, barely plants) having a phenotype of (1) reduced binding of a trichothecene mycotoxin to the RPL3 polypeptide encoded by a modified endogenous RPL3 gene, (2) increased tolerance to deoxynivalenol (DON), (3) increased resistance/tolerance to fusarium head blight (FHB) (e.g., increased resistance to the FHB causal agent, Fusarium graminearum and/or reduced symptoms of FHB), (4) reduced Fusarium graminearum load, and/or (5) producing seed that accumulates less trichothecene mycotoxin (e.g., DON) when infected with Fusarium graminearum, the method comprising planting two or more plants of the invention (e.g., 2, 3, 4, 5, 6, 7, 8, 9, 10, 20, 30, 40, 50, 100, 200, 300, 400, 500, 1000, 2000, 3000, 400, 5000, or 10,000 or more plants of the invention in a growing area (e.g., a field (e.g., a cultivated field, an agricultural field), a growth chamber, a greenhouse, a recreational area, a lawn, and/or a roadside and the like), thereby providing a plurality of plants having a phenotype of (1) reduced binding of a trichothecene mycotoxin to the RPL3 polypeptide encoded by a modified endogenous RPL3 gene, (2) increased tolerance to deoxynivalenol (DON), (3) increased resistance/tolerance to fusarium head blight (FHB) (e.g., increased resistance to the FHB causal agent, Fusarium graminearum and/or reduced symptoms of FHB), (4) reduced Fusarium graminearum load, and/or (5) producing seed that accumulates less trichothecene mycotoxin (e.g., DON) when infected with Fusarium graminearum as compared to a plurality of control plants devoid of the mutation.

In some embodiments, a method is provided for creating a mutation in an endogenous Ribosomal Protein L3 (RPL3) gene in a plant, comprising: (a) targeting a gene editing system to a region of the endogenous RPL3 gene that comprises a sequence having at least 90% sequence identity to any one of SEQ ID NOs:89-139; and (b) selecting a plant that comprises a modification located in the region of the endogenous RPL3 gene having at least 90% sequence identity to any one of SEQ ID NOs:89-139. In some embodiments, the modification is an out-of-frame deletion or out-of-frame insertion, optionally resulting in a truncated Ribosomal Protein L3 (RPL3) polypeptide, optionally resulting in no or little detectable RPL3 polypeptide. In some embodiments, the mutation may result in a mutated RPL3 gene having at least 90% sequence identity to any one of SEQ ID NOs:147, 149, 151, 153, or 155 and/or encoding a mutated RPL3 polypeptide having at least 90% sequence identity to any one of SEQ ID NOs:148, 150, 152, 154, or 156.

In some embodiments, a method of generating variation in a Ribosomal Protein L3 (RPL3) gene is provided, the method comprising: introducing an editing system into a plant cell, wherein the editing system is targeted to a region of a RPL3 gene that encodes a RPL3 polypeptide and contacting the region of the RPL3 gene with the editing system, thereby introducing a mutation into the RPL3 gene and generating variation in the RPL3 gene of the plant cell. In some embodiments, the RPL3 gene comprises a nucleotide sequence having at least 80% sequence identity to any one of SEQ ID NO:72, 73, 75, 76, 78, 79, 80, 81, 83, 84, 86, or 87, and/or encodes an amino acid sequence having at least 80% sequence identity to SEQ ID NO:74, 77, 82, 85, or 88, optionally wherein the region of the RPL3 gene that is targeted comprises at least 80% sequence identity to a nucleotide sequence of any one of SEQ ID NOs:89-139. In some embodiments, contacting the region of the endogenous RPL3 gene in the plant cell with the editing system produces a plant cell comprises in its genome an edited endogenous RPL3 gene, the method further comprising (a) regenerating a plant from the plant cell; (b) selfing the plant to produce progeny plants (E1); (c) assaying the progeny plants of (b) for increased resistance to fusarium head blight (FHB), a reduced Fusarium graminearum load, increased tolerance to deoxynivalenol (DON) and/or the ability to produce seeds that accumulate less trichothecene mycotoxin (DON); and (d) selecting the progeny plants exhibiting increased resistance to fusarium head blight (FHB), a reduced F. graminearum load, increased tolerance to deoxynivalenol (DON) and/or the ability to produce seeds that accumulate less trichothecene mycotoxin (DON) to produce selected progeny plants exhibiting increased resistance to fusarium head blight (FHB), a reduced F. graminearum load, increased tolerance to deoxynivalenol (DON) and/or the ability to produce seeds that accumulate less trichothecene mycotoxin (DON) as compared to a control plant. In some embodiments, the method further comprises (e) selfing the selected progeny plants of (d) to produce progeny plants (E2); (f) assaying the progeny plants of (e) for increased resistance to fusarium head blight (FHB), a reduced Fusarium graminearum load, increased tolerance to deoxynivalenol (DON) and/or the ability to produce seeds that accumulate less trichothecene mycotoxin (DON); and (g) selecting the progeny plants for increased resistance to fusarium head blight (FHB), a reduced Fusarium graminearum load, increased tolerance to deoxynivalenol (DON) and/or the ability to produce seeds that accumulate less trichothecene mycotoxin (DON) to produce selected progeny plants exhibiting for increased resistance to fusarium head blight (FHB), a reduced Fusarium graminearum load, increased tolerance to deoxynivalenol (DON) and/or the ability to produce seeds that accumulate less trichothecene mycotoxin (DON) as compared to a control plant, optionally repeating (e) through (g) one or more additional times. In some embodiments, the edit may result in a mutated RPL3 gene having at least 90% sequence identity to any one of SEQ ID NOs:147, 149, 151, 153, or 155 and/or encoding a mutated RPL3 polypeptide having at least 90% sequence identity to any one of SEQ ID NOs:148, 150, 152, 154, or 156.

In some embodiments, a method of detecting a mutant RPL3 gene (a mutation in an endogenous RPL3 gene, e.g., RPL3A and/or RPL3B) in a plant or plant part (e.g., plant cell) is provided, the method comprising detecting in the genome of the plant a RPL3 gene having at least one mutation within a region having at least 80% sequence identity (e.g., at least about 80, 81, 82, 83, 84, 85, 86, 87, 88, 89, 90, 91, 92, 93, 94, 95, 96, 97, 99 or 100% sequence identity) to a nucleotide sequence of any one of SEQ ID NOs:89-139. In some embodiments, a method of detecting a mutant Ribosomal Protein L3 (RPL3) gene is provided, the method comprising detecting in the genome of a plant an endogenous mutated RPL3 gene encoding a truncated polypeptide, optionally wherein the mutation is located in the 5′ region (optionally, within or adjacent to Exon 3) of the RPL3 gene having at least 80% sequence identity to any one of the nucleotide sequences of SEQ ID NOs:89-139. In some embodiments, the mutation that is detected is an out-of-frame deletion or an out-of-frame insertion. In some embodiments, a mutant RPL3 gene that is detected comprises a nucleic acid sequence having at least 90% sequence identity to any one of SEQ ID NOs:147, 149, 151, 153, or 155 and/or encodes a mutated RPL3 polypeptide having at least 90% sequence identity to any one of SEQ ID NOs:148, 150, 152, 154, or 156.

In some embodiments, a method for editing a specific site in the genome of a plant cell is provided, the method comprising: cleaving, in a site-specific manner, a target site within an endogenous Ribosomal Protein L3 (RPL3) gene in the plant cell, the endogenous RPL3 gene: (a) comprising a nucleotide sequence having at least 80% sequence identity to any one of SEQ ID NO:72, 73, 75, 76, 78, 79, 80, 81, 83, 84, 86, or 87, (b) comprising a region having at least 80% sequence identity to any one of the nucleotide sequences of SEQ ID NOs:89-139, and/or (c) encoding an amino acid sequence having at least 80% sequence identity to SEQ ID NO:74, 77, 82, 85, or 88, thereby generating an edit in the endogenous RPL3 gene of the plant cell and producing a plant cell comprising the edit in the endogenous RPL3 gene. In some embodiments, the endogenous RPL3 gene is an endogenous RPL3A gene and/or an endogenous RPL3B gene (optionally, a RPL3A-1 gene, a RPL3A-2 gene, a RPL3A-3 gene, a RPL3B-1 gene, a RPL3B-2 gene, and/or a RPL3B-3 gene), optionally wherein an edit is generated in two or more endogenous RPL3 genes (e.g., two or more of RPL3A gene, a RPL3A-1 gene, a RPL3A-2 gene, a RPL3A-3 gene, a RPL3B gene, a RPL3B-1 gene, a RPL3B-2 gene, and/or a RPL3B-3 gene). In some embodiments, the plant cell may be from a wheat plant, a spelt plant, a barley plant, or an oat plant, optionally, wherein the plant cell is from a wheat plant.

In some embodiments, the edit in the endogenous RPL3 gene results in a mutation including, but not limited to, a base deletion, a base substitution, or a base insertion. In some embodiments, the at least one mutation may be in the 5′ region of a RPL3 gene, for example, in or near/adjacent to the third exon of a RPL3 genomic sequence, wherein “near” or “adjacent to” refers to a region within about 1 to about 110 consecutive nucleotides of the third exon. In some embodiments, the edit may result in at least one mutation that is an insertion of at least one base pair (e.g., 1, 2, 3, 4, 5, 6, 7, 8, 9, 10, 11, 12, 13, 14, 15 or 16 base pairs, e.g., 1 to about 16 base pairs). In some embodiments, the edit may result in at least one mutation that is a deletion, optionally wherein the deletion is about 1 to about 100 consecutive base pairs in length, e.g., 1 to about 50 consecutive base pairs, about 1 to about 30 consecutive base pairs or about 1 to about 15 consecutive base pairs in length. A deletion or insertion useful with this invention may be an out-of-frame insertion or an out-of-frame deletion. In some embodiments, an out-of-frame insertion or out-of-frame deletion may result in a premature stop codon and truncated protein, optionally wherein the out-of-frame insertion or out-of-frame deletion results in no or little detectable protein (e.g., a knock-out or null mutation). In some embodiments, the edit in a RPL3 gene results in a truncated RPL3 polypeptide, optionally a C-terminal truncation of the RPL3 polypeptide, optionally wherein the C-terminal truncation results in a deletion of about 50 amino acid residues to about 300 amino acid residues (e.g., about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 1699, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 255, 260, 270, 275, 280, 285, 290, 295, or 300, and any range or value therein) from the C-terminus of the RPL3 polypeptide. In some embodiments, the mutation may be a non-natural mutation. In some embodiments, an edit may result in a mutated RPL3 gene having at least 90% sequence identity to any one of SEQ ID NOs:147, 149, 151, 153, or 155 and/or encoding a mutated RPL3 polypeptide having at least 90% sequence identity to any one of SEQ ID NOs:148, 150, 152, 154, or 156.

In some embodiments, a method of editing may further comprise regenerating a plant from the plant cell comprising the edit in the endogenous RPL3 gene, thereby producing a plant comprising the edit in its endogenous RPL3 gene (optionally in the 5′ end of the RPL3 gene, optionally in or near/adjacent to the third exon) and having a phenotype of one or more improved yield traits when compared to a control plant that is devoid of the edit.

In some embodiments, a method for making a plant is provided, the method comprising (a) contacting a population of plant cells comprising an endogenous Ribosomal Protein L3 (RPL3) gene with a nuclease linked to a nucleic acid binding domain (e.g., editing system) that binds to a sequence (i) having at least 80% sequence identity to any one of the nucleotide sequences of SEQ ID NO:72, 73, 75, 76, 78, 79, 80, 81, 83, 84, 86, or 87, (ii) comprising a region having at least 80% sequence identity to a nucleotide sequence of any one of SEQ ID NOs:89-139, and/or (iii) encoding an amino acid sequence having at least 80% sequence identity to SEQ ID NO:74, 77, 82, 85, or 88; (b) selecting a plant cell from the population of plant cells in which an endogenous RPL3 gene has been mutated, thereby producing a plant cell comprising a mutation in the endogenous RPL3 gene; and (c) growing the selected plant cell into a plant comprising the mutation in the endogenous gene encoding a RPL3 polypeptide, optionally wherein the mutation reduces or eliminates the ability of the RPL3 polypeptide to bind a trichothecene mycotoxin (e.g., deoxynivalenol (DON)), increases tolerance to deoxynivalenol (DON), increases resistance/tolerance to fusarium head blight (FHB) (e.g., increased resistance to the FHB causal agent, Fusarium graminearum and/or reduced symptoms of FHB infection), reduces Fusarium graminearum load and/or confers the ability of the plant to produce seed that accumulates less trichothecene mycotoxin (e.g., DON) when infected with Fusarium graminearum as compared to a plant devoid of the at least one mutation (e.g., an isogenic plant (e.g., wild type unedited plant or a null segregant).

In some embodiments, a method is provided for increasing resistance to Fusarium head blight (FHB) (e.g., increased resistance to the FHB causal agent, Fusarium graminearum and/or reduced symptoms of FHB infection (e.g., reduced FHB symptoms when infected with Fusarium graminearum)), increased tolerance to a trichothecene mycotoxin, optionally deoxynivalenol (DON), reduced Fusarium graminearum load and/or conferring the ability to produce seed that accumulates less trichothecene mycotoxin (e.g., DON) when infected with Fusarium graminearum in a plant, comprising (a) contacting a plant cell comprising a endogenous Ribosomal Protein L3 (RPL3) gene with a nuclease targeted to the endogenous RPL3 gene, wherein the nuclease is linked to a nucleic acid binding domain (e.g., editing system) that binds to a target site in the endogenous RPL3 gene and the endogenous RPL3 gene: (i) comprises a nucleotide sequence having at least 80% sequence identity to any one of SEQ ID NO:72, 73, 75, 76, 78, 79, 80, 81, 83, 84, 86, or 87, (ii) comprises a region having at least 80% sequence identity to any one of the nucleotide sequences of SEQ ID NOs:89-139, and/or (iii) encodes an amino acid sequence having at least 80% sequence identity to SEQ ID NO:74, 77, 82, 85, or 88, thereby producing a plant cell comprising a mutation in the endogenous RPL3 gene; and (b) growing the plant cell into a plant comprising the mutation in the endogenous RPL3 gene, thereby increasing resistance to FHB, increasing tolerance to the trichothecene mycotoxin, reducing Fusarium graminearum load in the plant and/or conferring the ability to produce seed that accumulates less trichothecene mycotoxin (e.g., DON) when infected with Fusarium graminearum to the plant.

In some embodiments, a method for producing a plant or part thereof comprising at least one cell having a mutated Ribosomal Protein L3 (RPL3) gene is provided, the method comprising contacting a target site in an endogenous RPL3 gene in the plant or plant part with a nuclease comprising a cleavage domain and a nucleic acid binding domain, wherein the nucleic acid binding domain binds to a target site in the endogenous RPL3 gene, wherein the endogenous RPL3 gene (a) comprises a sequence having at least 80% sequence identity to any one of the nucleotide sequences of SEQ ID NO:72, 73, 75, 76, 78, 79, 80, 81, 83, 84, 86, or 87; (b) comprises a region having at least 80% identity to any one of SEQ ID NOs:89-139; and/or (c) encodes an amino acid sequence having at least 80% sequence identity to SEQ ID NO:74, 77, 82, 85, or 88, thereby producing the plant or part thereof comprising at least one cell having a mutation in the endogenous RPL3 gene (e.g., one or more mutated endogenous RPL3 genes), optionally wherein the mutation in the endogenous RPL3 gene produces a RPL3 polypeptide having reduced binding to a trichothecene mycotoxin, and/or the plant comprising at least one cell having a mutation in the endogenous RPL3 gene exhibits (i) increased tolerance to deoxynivalenol (DON), (ii) increased resistance/tolerance to fusarium head blight (FHB) (e.g., increased resistance to the FHB causal agent, Fusarium graminearum and/or reduced symptoms of FHB infection (e.g., reduced FHB symptoms when infected with Fusarium graminearum)),(iii) increased tolerance to a trichothecene mycotoxin, optionally deoxynivalenol (DON), (iv) reduced Fusarium graminearum load and/or exhibits the ability to produce seed that accumulates less trichothecene mycotoxin (e.g., DON) when infected with Fusarium graminearum as compared to a plant devoid of the at least one mutation (e.g., an isogenic plant (e.g., wild type unedited plant or a null segregant).

Also provided herein is a method of producing a plant or part thereof comprising a mutation in an endogenous RPL3 gene that produces a mutated RPL3 polypeptide having reduced binding to a trichothecene mycotoxin, the method comprising contacting a target site in the endogenous RPL3 gene in the plant or plant part with a nuclease comprising a cleavage domain and a nucleic acid binding domain, wherein the nucleic acid binding domain of the nuclease binds to a target site in the endogenous RPL3 gene, wherein the endogenous RPL3 gene: (a) comprises a sequence having at least 80% sequence identity to any one of the nucleotide sequences of SEQ ID NO:72, 73, 75, 76, 78, 79, 80, 81, 83, 84, 86, or 87; (b) comprises a region having at least 80% identity to any one of SEQ ID NOs:89-139; and/or (c) encodes an amino acid sequence having at least 80% sequence identity to SEQ ID NO:74, 77, 82, 85, or 88, thereby producing a plant or part thereof having a mutation in the endogenous RPL3 gene that produces a mutated RPL3 polypeptide having reduced binding to the trichothecene mycotoxin. In some embodiments, the plant or part thereof having a mutation in the endogenous RPL3 gene that produces a mutated RPL3 polypeptide having reduced binding to the trichothecene mycotoxin further exhibits a phenotype of (1) increased tolerance to deoxynivalenol (DON), (2) increased resistance/tolerance to fusarium head blight (FHB) (e.g., increased resistance to the FHB causal agent, Fusarium graminearum and/or reduced symptoms of FHB), (3) reduced Fusarium graminearum load, and/or (4) producing seed that accumulates less trichothecene mycotoxin (e.g., DON) when infected with Fusarium graminearum.

In some embodiments of the invention, the nuclease cleaves the endogenous RPL3 gene, thereby introducing the mutation into the endogenous RPL3 gene. In some embodiments, the mutation is a non-natural mutation. In some embodiments, the mutation may be a base substitution, a base deletion and/or a base insertion. In some embodiments, the mutation may be point mutation. In some embodiments, the mutation may be a knockout mutation (e.g., the gene is inoperative, or there is no gene expression) or a knockdown mutation (e.g., reduced expression, or reduced functionality).

In some embodiments, the mutation is a null mutation. In some embodiments, the mutation may be an insertion, optionally an out-of-frame insertion. In some embodiments, the mutation may be a deletion, optionally an out-of-frame deletion (e.g., a deletion of one to about 100 base pairs). In some embodiments, the mutation results in a premature stop codon, optionally resulting in a truncated protein.

In some embodiments, a plant having a mutation in the endogenous RPL3 gene as described herein may comprise a mutated RPL3 polypeptide having reduced binding to a trichothecene mycotoxin, and/or may exhibit: increased tolerance to deoxynivalenol (DON), increased resistance/tolerance to fusarium head blight (FHB) (e.g., increased resistance/tolerance to the FHB causal agent, Fusarium graminearum, and/or reduced symptoms of FHB), a reduced Fusarium graminearum load, and/or the ability to produce seed that accumulates less trichothecene mycotoxin (e.g., DON) when infected with Fusarium graminearum as compared to a control plant that is devoid of the mutation in the endogenous RPL3 gene.

In some embodiments, a nuclease may cleave an endogenous RPL3 gene, thereby introducing the mutation into the endogenous RPL3 gene. A nuclease useful with the invention may be any nuclease that can be utilized to edit/modify a target nucleic acid. Such nucleases include, but are not limited to a zinc finger nuclease, transcription activator-like effector nucleases (TALEN), endonuclease (e.g., Fok1) and/or a CRISPR-Cas effector protein. Likewise, any nucleic acid binding domain useful with the invention may be any DNA binding domain or RNA binding domain that can be utilized to edit/modify a target nucleic acid. Such nucleic acid binding domains include, but are not limited to, a zinc finger, transcription activator-like DNA binding domain (TAL), an argonaute and/or a CRISPR-Cas effector DNA binding domain.

In some embodiments, the methods of the invention may result in a mutated RPL3 gene having at least 90% sequence identity to any one of SEQ ID NOs:147, 149, 151, 153, or 155 and/or that encodes a mutated RPL3 polypeptide having at least 90% sequence identity to any one of SEQ ID NOs:148, 150, 152, 154, or 156.

A “nucleic acid binding domain” as used herein refers to a polypeptide or domain that binds or is capable of binding a nucleic acid (e.g., a target nucleic acid). A nucleic acid binding domain may be a site- and/or sequence-specific nucleic acid binding domain. A DNA binding domain is an example nucleic acid binding domain, which may be a site- and/or sequence-specific nucleic acid binding domain. In some embodiments, a nucleic acid binding domain (e.g., DNA binding domain) is comprised in a nucleic acid binding polypeptide. A “nucleic acid binding protein” or “nucleic acid binding polypeptide” as used herein refers to a polypeptide that binds and/or is capable of binding a nucleic acid in a site- and/or sequence-specific manner. In some embodiments, a nucleic acid binding domain may be a sequence-specific nucleic acid binding domain such as, but not limited to, a sequence-specific binding domain from, for example, a polynucleotide-guided endonuclease, a CRISPR-Cas effector protein (e.g., a CRISPR-Cas endonuclease), a zinc finger nuclease, a transcription activator-like effector nuclease (TALEN) and/or an Argonaute protein. In some embodiments, a nucleic acid binding polypeptide comprises a cleavage domain (e.g., a nuclease domain) such as, but not limited to, an endonuclease (e.g., Fok1), a polynucleotide-guided endonuclease, a CRISPR-Cas endonuclease, a zinc finger nuclease, and/or a transcription activator-like effector nuclease (TALEN). In some embodiments, the nucleic acid binding domain is a polypeptide that can associate (e.g., form a complex) with one or more nucleic acid molecule(s) (e.g., form a complex with a guide nucleic acid as described herein) that can direct or guide the nucleic acid binding domain to a specific target nucleotide sequence (e.g., a gene locus of a genome) that is complementary to the one or more nucleic acid molecule(s) (or a portion or region thereof), thereby causing the nucleic acid binding domain to bind to the nucleotide sequence at the specific target site. In some embodiments, the nucleic acid binding domain is a CRISPR-Cas effector protein as described herein. In some embodiments, a polynucleotide and/or a nucleic acid construct of the invention can be an “expression cassette” or can be comprised within an expression cassette.

In some embodiments, a method of editing an endogenous Ribosomal Protein L3 (RPL3) gene (e.g., RPL3A and/or RPL3B) in a plant or plant part is provided, the method comprising contacting a target site in an endogenous RPL3 gene in the plant or plant part with a cytosine base editing system comprising a cytosine deaminase and a nucleic acid binding domain that binds to a target site in the endogenous RPL3 gene, wherein the endogenous RPL3 gene: (a) comprises a sequence having at least 80% sequence identity to a nucleotide sequence of any one of SEQ ID NO:72, 73, 75, 76, 78, 79, 80, 81, 83, 84, 86, or 87; (b) comprises a region having at least 80% identity to any one of SEQ ID NOs:89-139; and/or (c) encodes an amino acid sequence having at least 80% sequence identity to any one of SEQ ID NO:74, 77, 82, 85, or 88, thereby editing the endogenous RPL3 gene in the plant or part thereof and producing a plant or part thereof comprising at least one cell having a mutation in the endogenous RPL3 gene.

In some embodiments, a method of editing an endogenous Ribosomal Protein L3 (RPL3) gene (e.g., RPL3A and/or RPL3B) in a plant or plant part is provided, the method comprising contacting a target site in an endogenous RPL3 gene in the plant or plant part with an adenosine base editing system comprising an adenosine deaminase and a nucleic acid binding domain that binds to a target site in the endogenous RPL3 gene, wherein the endogenous RPL3 gene: (a) comprises a sequence having at least 80% sequence identity to a nucleotide sequence of any one of SEQ ID NO:72, 73, 75, 76, 78, 79, 80, 81, 83, 84, 86, or 87; (b) comprises a region having at least 80% identity to any one of SEQ ID NOs:89-139; and/or (c) encodes an amino acid sequence having at least 80% sequence identity to any one of SEQ ID NO:74, 77, 82, 85, or 88, thereby editing the endogenous RPL3 gene in the plant or part thereof and producing a plant or part thereof comprising at least one cell having a mutation in the endogenous RPL3 gene.

In some embodiments, the mutation may be a substitution, an insertion and/or a deletion, optionally wherein the insertion or deletion is an out-of-frame insertion or an out-of-frame deletion. In some embodiments, a mutation may comprise a base substitution to an A, a T, a G, or a C. In some embodiments, the mutation may be a non-natural mutation. In some embodiments, the mutation may be a deletion (e.g., out-of-frame deletion) of about 1 base pair to about 100 consecutive base pairs, optionally, 1 to about 50 consecutive base pairs, 1 to about 30 consecutive base pairs, or 1 to about 15 consecutive base pairs. In some embodiments, the mutation may be an insertion (e.g., an out-of-frame insertion) of at least one base pair (e.g., 1 base pair to about 16 base pairs; e.g., 1, 2, 3, 4, 5, 6, 7, 8, 9, 10, 11, 12, 13, 14, 15 or 16 consecutive base pairs, optionally 1, 2, 4, 5, 7, 8, 9, 10, 11, 13, or 14 consecutive base pairs). A mutation in a RPL3 gene may be located in the 5′ region of the RPL3 gene, optionally wherein the mutation may be within a portion or region of the endogenous RPL3 gene that encodes the RPL3 polypeptide (e.g., the coding regions (exons)). In some embodiments, the mutation in a RPL3 gene may be located in or adjacent to Exon 3 of the RPL3 gene. As used herein, “in or adjacent to Exon 3” means within 1 to about 110 consecutive nucleotides of the 5′ or 3′ region of Exon 3 of the RPL3 gene (e.g., within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 consecutive nucleotides 5′ or 3′ adjacent to Exon 3). In some embodiments, a mutation of a RPL3 gene that is an out-of-frame deletion or an out-of-frame insertion may result in a truncated polypeptide and/or may result in little or no detectible polypeptide. In some embodiments, the out-of-frame deletion or out-of-frame insertion may be a null mutation. In some embodiments, an out-of-frame deletion or an out-of-frame insertion may be located in the 5′ region of the RPL3 gene (e.g., in or adjacent to Exon 3) that results in a premature stop codon (e.g., an out-of-frame base insertion or an out-of-frame base deletion) and a truncated RPL3 polypeptide, optionally resulting in little or no detectable RPL3 polypeptide, optionally a resulting in a non-functional RPL3 polypeptide.

In some embodiments, the methods of the invention may result in a mutated RPL3 gene having at least 90% sequence identity to any one of SEQ ID NOs:147, 149, 151, 153, or 155 and/or that encodes a mutated RPL3 polypeptide having at least 90% sequence identity to any one of SEQ ID NOs:148, 150, 152, 154, or 156.

In some embodiments, the present invention provides a method of producing a plant comprising a mutation in an endogenous Ribosomal Protein L3 (RPL3) gene (e.g., a RPL3A gene, a RPL3A-1 gene, a RPL3A-2 gene, a RPL3A-3 gene, a RPL3B gene, a RPL3B-1 gene, a RPL3B-2 gene, and/or a RPL3B-3 gene) and at least one polynucleotide of interest, the method comprising crossing a plant of the invention comprising at least one mutation in an endogenous RPL3 gene (a first plant) with a second plant that comprises the at least one polynucleotide of interest to produce progeny plants; and selecting progeny plants comprising at least one mutation in the RPL3 gene and the at least one polynucleotide of interest, thereby producing the plant comprising a mutation in an endogenous RPL3 gene and at least one polynucleotide of interest.

The present invention further provides a method of producing a plant comprising a mutation in an endogenous RPL3 gene (e.g., a RPL3A gene, a RPL3A-1 gene, a RPL3A-2 gene, a RPL3A-3 gene, a RPL3B gene, a RPL3B-1 gene, a RPL3B-2 gene, and/or a RPL3B-3 gene) and at least one polynucleotide of interest, the method comprising introducing at least one polynucleotide of interest into a plant of the present invention comprising at least one mutation in a RPL3 gene, thereby producing a plant comprising at least one mutation in a RPL3 gene and at least one polynucleotide of interest. In some embodiments, the plant is a wheat plant, an oat plant, a barley plant, a spelt plant. In some embodiments, the plant is a wheat plant.

In some embodiments, also provided is a method of producing a plant comprising a mutation in an endogenous RPL3 gene (e.g., a RPL3A gene, a RPL3A-1 gene, a RPL3A-2 gene, a RPL3A-3 gene, a RPL3B gene, a RPL3B-1 gene, a RPL3B-2 gene, and/or a RPL3B-3 gene) and exhibiting a phenotype of improved yield traits, improved plant architecture and/or improved defense traits, the method comprising crossing a first plant, which is a plant of the present invention comprising at least one mutation in a RPL3 gene, with a second plant that exhibits a phenotype of improved yield traits, improved plant architecture and/or improved defense traits; and selecting progeny plants comprising the mutation in the RPL3 gene and a phenotype of improved yield traits, improved plant architecture and/or improved defense traits, thereby producing the plant comprising a mutation in an endogenous RP3 gene and exhibiting a phenotype of improved yield traits, improved plant architecture and/or improved defense traits as compared to a control plant.

Further provided is a method of controlling weeds in a container (e.g., pot, or seed tray and the like), a growth chamber, a greenhouse, a field, a recreational area, a lawn, or on a roadside, the method comprising applying an herbicide to one or more (a plurality) plants of the invention (e.g., a plant comprising at least one mutation in a RPL3 gene (e.g., a RPL3A gene, a RPL3A-1 gene, a RPL3A-2 gene, a RPL3A-3 gene, a RPL3B gene, a RPL3B-1 gene, a RPL3B-2 gene, and/or a RPL3B-3 gene)) as described herein) growing in a container, a growth chamber, a greenhouse, a field, a recreational area, a lawn, or on a roadside, thereby controlling the weeds in the container, the growth chamber, the greenhouse, the field, the recreational area, the lawn, or on the roadside in which the one or more plants are growing.

In some embodiments, a method of reducing insect predation on a plant is provided, the method comprising applying an insecticide to one or more plants of the invention, optionally, wherein the one or more plants are growing in a container, a growth chamber, a greenhouse, a field, a recreational area, a lawn, or on a roadside, thereby reducing insect predation on the one or more plants.

In some embodiments, a method of reducing fungal disease on a plant is provided, the method comprising applying a fungicide to one or more plants of the invention, optionally, wherein the one or more plants are growing in a container, a growth chamber, a greenhouse, a field, a recreational area, a lawn, or on a roadside, thereby reducing fungal disease on the one or more plants.

A polynucleotide of interest may be any polynucleotide that can confer a desirable phenotype or otherwise modify the phenotype or genotype of a plant. In some embodiments, a polynucleotide of interest may be polynucleotide that confers herbicide tolerance, insect resistance, nematode resistance, disease resistance, increased yield, increased nutrient use efficiency or abiotic stress resistance.

Thus, plants or plant cultivars which are to be treated with preference in accordance with the invention include all plants which, through genetic modification, received genetic material which imparts particular advantageous useful properties (“traits”) to these plants. Examples of such properties are better plant growth, vigor, stress tolerance, standability, lodging resistance, nutrient uptake, plant nutrition, and/or yield, in particular improved growth, increased tolerance to high or low temperatures, increased tolerance to drought or to levels of water or soil salinity, enhanced flowering performance, easier harvesting, accelerated ripening, higher yields, higher quality and/or a higher nutritional value of the harvested products, better storage life and/or processability of the harvested products.

Further examples of such properties are an increased resistance against animal and microbial pests, such as against insects, arachnids, nematodes, mites, slugs and snails owing, for example, to toxins formed in the plants. Among DNA sequences encoding proteins which confer properties of tolerance to such animal and microbial pests, in particular insects, mention will particularly be made of the genetic material from Bacillus thuringiensis encoding the Bt proteins widely described in the literature and well known to those skilled in the art. Mention will also be made of proteins extracted from bacteria such as Photorhabdus (WO97/17432 and WO98/08932). In particular, mention will be made of the Bt Cry or VIP proteins which include the CrylA, CryIAb, CryIAc, CryIIA, CryIIIA, CryIIIB2, Cry9c Cry2Ab, Cry3Bb and CryIF proteins or toxic fragments thereof and also hybrids or combinations thereof, especially the CrylF protein or hybrids derived from a CrylF protein (e.g. hybrid CrylA-CrylF proteins or toxic fragments thereof), the CrylA-type proteins or toxic fragments thereof, preferably the CrylAc protein or hybrids derived from the CrylAc protein (e.g. hybrid CrylAb-CrylAc proteins) or the CrylAb or Bt2 protein or toxic fragments thereof, the Cry2Ae, Cry2Af or Cry2Ag proteins or toxic fragments thereof, the Cry1A.105 protein or a toxic fragment thereof, the VIP3Aa19 protein, the VIP3Aa20 protein, the VIP3A proteins produced in the COT202 or COT203 cotton events, the VIP3Aa protein or a toxic fragment thereof as described in Estruch et al. (1996), Proc Natl Acad Sci US A. 28;93(11):5389-94, the Cry proteins as described in WO2001/47952, the insecticidal proteins from Xenorhabdus (as described in y), Serratia (particularly from S. entomophila) or Photorhabdus species strains, such as Tc-proteins from Photorhabdus as described in WO98/08932. Also any variants or mutants of any one of these proteins differing in some amino acids (1-10, preferably 1-5) from any of the above named sequences, particularly the sequence of their toxic fragment, or which are fused to a transit peptide, such as a plastid transit peptide, or another protein or peptide, is included herein.

Another and particularly emphasized example of such properties is conferred tolerance to one or more herbicides, for example imidazolinones, sulphonylureas, glyphosate or phosphinothricin. Among DNA sequences encoding proteins (i.e., polynucleotides of interest) which confer properties of tolerance to certain herbicides on the transformed plant cells and plants, mention will be particularly be made to the bar or PAT gene or the Streptomyces coelicolor gene described in WO2009/152359 which confers tolerance to glufosinate herbicides, a gene encoding a suitable EPSPS (5-Enolpyruvylshikimat-3-phosphat-Synthase) which confers tolerance to herbicides having EPSPS as a target, especially herbicides such as glyphosate and its salts, a gene encoding glyphosate-n-acetyltransferase, or a gene encoding glyphosate oxidoreductase. Further suitable herbicide tolerance traits include at least one ALS (acetolactate synthase) inhibitor (e.g., WO2007/024782), a mutated Arabidopsis ALS/AHAS gene (e.g., U.S. Pat. 6,855,533), genes encoding 2,4-D-monooxygenases conferring tolerance to 2,4-D (2,4-dichlorophenoxyacetic acid) and genes encoding Dicamba monooxygenases conferring tolerance to dicamba (3,6-dichloro-2- methoxybenzoic acid).

Further examples of such properties are increased resistance against phytopathogenic fungi, bacteria and/or viruses owing, for example, to systemic acquired resistance (SAR), systemin, phytoalexins, elicitors and also resistance genes and correspondingly expressed proteins and toxins.

Particularly useful transgenic events in transgenic plants or plant cultivars which can be treated with preference in accordance with the invention include Event 531/ PV-GHBK04 (cotton, insect control, described in WO2002/040677), Event 1143-14A (cotton, insect control, not deposited, described in WO2006/128569); Event 1143-51B (cotton, insect control, not deposited, described in WO2006/128570); Event 1445 (cotton, herbicide tolerance, not deposited, described in US-A 2002-120964 or WO2002/034946); Event 17053 (rice, herbicide tolerance, deposited as PTA-9843, described in WO2010/117737); Event 17314 (rice, herbicide tolerance, deposited as PTA-9844, described in WO2010/117735); Event 281-24-236 (cotton, insect control - herbicide tolerance, deposited as PTA-6233, described in WO2005/103266 or US-A 2005-216969); Event 3006-210-23 (cotton, insect control - herbicide tolerance, deposited as PTA-6233, described in US-A 2007-143876 orWO2005/103266); Event 3272 (corn, quality trait, deposited as PTA-9972, described in WO2006/098952 or US-A 2006-230473); Event 33391 (wheat, herbicide tolerance, deposited as PTA-2347, described in WO2002/027004), Event 40416 (corn, insect control - herbicide tolerance, deposited as ATCC PTA-11508, described in WO 11/075593); Event 43A47 (corn, insect control - herbicide tolerance, deposited as ATCC PTA-11509, described in WO2011/075595); Event 5307 (corn, insect control, deposited as ATCC PTA-9561, described in WO2010/077816); Event ASR-368 (bent grass, herbicide tolerance, deposited as ATCC PTA-4816, described in US-A 2006-162007 or WO2004/053062); Event B16 (corn, herbicide tolerance, not deposited, described in US-A 2003-126634); Event BPS-CV127- 9 (soybean, herbicide tolerance, deposited as NCIMB No. 41603, described in WO2010/080829); Event BLR1 (oilseed rape, restoration of male sterility, deposited as NCIMB 41193, described in WO2005/074671), Event CE43-67B (cotton, insect control, deposited as DSM ACC2724, described in US-A 2009-217423 or WO2006/128573); Event CE44-69D (cotton, insect control, not deposited, described in US-A 2010- 0024077); Event CE44-69D (cotton, insect control, not deposited, described in WO2006/128571); Event CE46-02A (cotton, insect control, not deposited, described in WO2006/128572); Event COT102 (cotton, insect control, not deposited, described in US-A 2006-130175 or WO2004/039986); Event COT202 (cotton, insect control, not deposited, described in US-A 2007-067868 or WO2005/054479); Event COT203 (cotton, insect control, not deposited, described in WO2005/054480); ); Event DAS21606-3 / 1606 (soybean, herbicide tolerance, deposited as PTA-11028, described in WO2012/033794), Event DAS40278 (corn, herbicide tolerance, deposited as ATCC PTA-10244, described in WO2011/022469); Event DAS-44406-6 / pDAB8264.44.06.1 (soybean, herbicide tolerance, deposited as PTA-11336, described in WO2012/075426), Event DAS-14536-7 /pDAB8291.45.36.2 (soybean, herbicide tolerance, deposited as PTA-11335, described in WO2012/075429), Event DAS-59122-7 (corn, insect control – herbicide tolerance, deposited as ATCC PTA 11384, described in US-A 2006-070139); Event DAS-59132 (corn, insect control – herbicide tolerance, not deposited, described in WO2009/100188); Event DAS68416 (soybean, herbicide tolerance, deposited as ATCC PTA-10442, described in WO2011/066384 or WO2011/066360); Event DP-098140-6 (corn, herbicide tolerance, deposited as ATCC PTA-8296, described in US-A 2009- 137395 or WO 08/112019); Event DP-305423-1 (soybean, quality trait, not deposited, described in US-A 2008-312082 or WO2008/054747); Event DP-32138-1 (corn, hybridization system, deposited as ATCC PTA-9158, described in US-A 2009-0210970 or WO2009/103049); Event DP-356043-5 (soybean, herbicide tolerance, deposited as ATCC PTA-8287, described in US-A 2010-0184079 or WO2008/002872); Event EE-I (brinjal, insect control, not deposited, described in WO 07/091277); Event Fil 17 (corn, herbicide tolerance, deposited as ATCC 209031, described in US-A 2006-059581 or WO 98/044140); Event FG72 (soybean, herbicide tolerance, deposited as PTA-11041, described in WO2011/063413), Event GA21 (corn, herbicide tolerance, deposited as ATCC 209033, described in US-A 2005-086719 or WO 98/044140); Event GG25 (corn, herbicide tolerance, deposited as ATCC 209032, described in US-A 2005-188434 or WO98/044140); Event GHB119 (cotton, insect control - herbicide tolerance, deposited as ATCC PTA-8398, described in WO2008/151780); Event GHB614 (cotton, herbicide tolerance, deposited as ATCC PTA-6878, described in US-A 2010-050282 or W02007/017186); Event GJ11 (corn, herbicide tolerance, deposited as ATCC 209030, described in US-A 2005-188434 or WO98/044140); Event GM RZ13 (sugar beet, virus resistance, deposited as NCIMB-41601, described in WO2010/076212); Event H7-1 (sugar beet, herbicide tolerance, deposited as NCIMB 41158 or NCIMB 41159, described in US-A 2004-172669 or WO 2004/074492); Event JOPLIN1 (wheat, disease tolerance, not deposited, described in US-A 2008-064032); Event LL27 (soybean, herbicide tolerance, deposited as NCIMB41658, described in WO2006/108674 or US-A 2008-320616); Event LL55 (soybean, herbicide tolerance, deposited as NCIMB 41660, described in WO 2006/108675 or US-A 2008-196127); Event LLcotton25 (cotton, herbicide tolerance, deposited as ATCC PTA-3343, described in WO2003/013224 or US- A 2003-097687); Event LLRICE06 (rice, herbicide tolerance, deposited as ATCC 203353, described in US 6,468,747 or WO2000/026345); Event LLRice62 ( rice, herbicide tolerance, deposited as ATCC 203352, described in WO2000/026345), Event LLRICE601 (rice, herbicide tolerance, deposited as ATCC PTA-2600, described in US-A 2008-2289060 or WO2000/026356); Event LY038 (corn, quality trait, deposited as ATCC PTA-5623, described in US-A 2007-028322 or WO2005/061720); Event MIR162 (corn, insect control, deposited as PTA-8166, described in US-A 2009-300784 or WO2007/142840); Event MIR604 (corn, insect control, not deposited, described in US-A 2008-167456 or WO2005/103301); Event MON15985 (cotton, insect control, deposited as ATCC PTA-2516, described in US-A 2004-250317 or WO2002/100163); Event MON810 (corn, insect control, not deposited, described in US-A 2002-102582); Event MON863 (corn, insect control, deposited as ATCC PTA-2605, described in WO2004/011601 or US-A 2006-095986); Event MON87427 (corn, pollination control, deposited as ATCC PTA-7899, described in WO2011/062904); Event MON87460 (corn, stress tolerance, deposited as ATCC PTA-8910, described in WO2009/111263 or US-A 2011-0138504); Event MON87701 (soybean, insect control, deposited as ATCC PTA- 8194, described in US-A 2009-130071 or WO2009/064652); Event MON87705 (soybean, quality trait - herbicide tolerance, deposited as ATCC PTA-9241, described in US-A 2010-0080887 or WO2010/037016); Event MON87708 (soybean, herbicide tolerance, deposited as ATCC PTA-9670, described in WO2011/034704); Event MON87712 (soybean, yield, deposited as PTA-10296, described in WO2012/051199), Event MON87754 (soybean, quality trait, deposited as ATCC PTA-9385, described in WO2010/024976); Event MON87769 (soybean, quality trait, deposited as ATCC PTA- 8911, described in US-A 2011-0067141 or WO2009/102873); Event MON88017 (corn, insect control - herbicide tolerance, deposited as ATCC PTA-5582, described in US-A 2008-028482 or WO2005/059103); Event MON88913 (cotton, herbicide tolerance, deposited as ATCC PTA-4854, described in WO2004/072235 or US-A 2006-059590); Event MON88302 (oilseed rape, herbicide tolerance, deposited as PTA-10955, described in WO2011/153186), Event MON88701 (cotton, herbicide tolerance, deposited as PTA-11754, described in WO2012/134808), Event MON89034 (corn, insect control, deposited as ATCC PTA-7455, described in WO 07/140256 or US-A 2008-260932); Event MON89788 (soybean, herbicide tolerance, deposited as ATCC PTA-6708, described in US-A 2006-282915 or WO2006/130436); Event MS1 1 (oilseed rape, pollination control - herbicide tolerance, deposited as ATCC PTA-850 or PTA-2485, described in WO2001/031042); Event MS8 (oilseed rape, pollination control - herbicide tolerance, deposited as ATCC PTA-730, described in WO2001/041558 or US-A 2003-188347); Event NK603 (corn, herbicide tolerance, deposited as ATCC PTA-2478, described in US-A 2007-292854); Event PE-7 (rice, insect control, not deposited, described in WO2008/114282); Event RF3 (oilseed rape, pollination control -herbicide tolerance, deposited as ATCC PTA-730, described in WO2001/041558 or US-A 2003-188347); Event RT73 (oilseed rape, herbicide tolerance, not deposited, described in WO2002/036831 or US-A 2008-070260); Event SYHT0H2 / SYN-000H2-5 (soybean, herbicide tolerance, deposited as PTA-11226, described in WO2012/082548), Event T227-1 (sugar beet, herbicide tolerance, not deposited, described in WO2002/44407 or US-A 2009-265817); Event T25 (corn, herbicide tolerance, not deposited, described in US-A 2001-029014 or WO2001/051654); Event T304-40 (cotton, insect control - herbicide tolerance, deposited as ATCC PTA-8171, described in US-A 2010-077501 or WO2008/122406); Event T342-142 (cotton, insect control, not deposited, described in WO2006/128568); Event TC1507 (corn, insect control - herbicide tolerance, not deposited, described in US-A 2005-039226 or WO2004/099447); Event VIP1034 (corn, insect control - herbicide tolerance, deposited as ATCC PTA-3925, described in WO2003/052073), Event 32316 (corn, insect control-herbicide tolerance, deposited as PTA-11507, described in WO2011/084632), Event 4114 (corn, insect control-herbicide tolerance, deposited as PTA-11506, described in W02011/084621), event EE-GM3 / FG72 (soybean, herbicide tolerance, ATCC Accession N° PTA-11041) optionally stacked with event EE-GM1/LL27 or event EE-GM2/LL55 (WO2011/063413A2), event DAS-68416-4 (soybean, herbicide tolerance, ATCC Accession N° PTA-10442, WO2011/066360A1), event DAS-68416-4 (soybean, herbicide tolerance, ATCC Accession N° PTA-10442, WO2011/066384A1), event DP-040416-8 (corn, insect control, ATCC Accession N° PTA-11508, WO2011/075593A1), event DP-043A47-3 (corn, insect control, ATCC Accession N° PTA-11509, WO2011/075595A1), event DP- 004114-3 (corn, insect control, ATCC Accession N° PTA-11506, WO2011/084621A1), event DP-032316-8 (corn, insect control, ATCC Accession N° PTA-11507, WO2011/084632A1), event MON-88302-9 (oilseed rape, herbicide tolerance, ATCC Accession N° PTA-10955, WO2011/153186A1), event DAS-21606-3 (soybean, herbicide tolerance, ATCC Accession No. PTA-11028, WO2012/033794A2), event MON-87712-4 (soybean, quality trait, ATCC Accession N°. PTA-10296, WO2012/051199A2), event DAS-44406-6 (soybean, stacked herbicide tolerance, ATCC Accession N°. PTA-11336, WO2012/075426A1), event DAS-14536-7 (soybean, stacked herbicide tolerance, ATCC Accession N°. PTA-11335, WO2012/075429A1), event SYN-000H2-5 (soybean, herbicide tolerance, ATCC Accession N°. PTA-11226, WO2012/082548A2), event DP-061061-7 (oilseed rape, herbicide tolerance, no deposit N° available, WO2012071039A1), event DP-073496-4 (oilseed rape, herbicide tolerance, no deposit N° available, US2012131692), event 8264.44.06.1 (soybean, stacked herbicide tolerance, Accession N° PTA-11336, WO2012075426A2), event 8291.45.36.2 (soybean, stacked herbicide tolerance, Accession N°. PTA-11335, WO2012075429A2), event SYHT0H2 (soybean, ATCC Accession N°. PTA-11226, WO2012/082548A2), event MON88701 (cotton, ATCC Accession N° PTA-11754, WO2012/134808A1), event KK179-2 (alfalfa, ATCC Accession N° PTA-11833, WO2013/003558A1), event pDAB8264.42.32.1 (soybean, stacked herbicide tolerance, ATCC Accession N° PTA-11993, WO2013/010094A1), event MZDT09Y (corn, ATCC Accession N° PTA-13025, WO2013/012775A1).

The genes/events (e.g., polynucleotides of interest), which impart the desired traits in question, may also be present in combinations with one another in the transgenic plants. Examples of transgenic plants which may be mentioned are the important crop plants, such as cereals (wheat, rice, triticale, barley, rye, oats), maize, soya beans, potatoes, sugar beet, sugar cane, tomatoes, peas and other types of vegetable, cotton, tobacco, oilseed rape and also fruit plants (with the fruits apples, pears, citrus fruits and grapes), with particular emphasis being given to maize, soya beans, wheat, rice, potatoes, cotton, sugar cane, tobacco and oilseed rape. Traits which are particularly emphasized are the increased resistance of the plants to insects, arachnids, nematodes and slugs and snails, as well as the increased resistance of the plants to one or more herbicides.

Commercially available examples of such plants, plant parts or plant seeds that may be treated with preference in accordance with the invention include commercial products, such as plant seeds, sold or distributed under the GENUITY®, DROUGHTGARD®, SMARTSTAX®, RIB COMPLETE®, ROUNDUP READY®, VT DOUBLE PRO®, VT TRIPLE PRO®, BOLLGARD II®, ROUNDUP READY 2 YIELD®, YIELDGARD®, ROUNDUP READY® 2 XTENDTM, INTACTA RR2 PRO®, VISTIVE GOLD®, and/or XTENDFLEX™ trade names.

A Ribosomal Protein L3 (RPL3) gene (e.g., RPL3A and/or RPL3B) useful with this invention includes any endogenous RPL3 gene in which a mutation as described herein can confer one or more of the phenotypes of (1) reduced binding of a trichothecene mycotoxin to the RPL3 polypeptide encoded by the mutated RPL3 gene, (2) increased tolerance to deoxynivalenol (DON), (3) increased resistance/tolerance to fusarium head blight (FHB) (e.g., increased resistance to the FHB causal agent, Fusarium graminearum, and/or decreased FHB symptoms), (4) reduced Fusarium graminearum load, and/or (5) producing seed that accumulates less trichothecene mycotoxin (e.g., DON) when infected with Fusarium graminearum as compared to a plant devoid of the at least one mutation (e.g., an isogenic plant (e.g., wild type unedited plant or a null segregant).

In some embodiments, an endogenous RPL3 gene (a) comprises a sequence having at least 80% sequence identity to a nucleotide sequence of any one of SEQ ID NO:72, 73, 75, 76, 78, 79, 80, 81, 83, 84, 86, or 87; (b) comprises a region having at least 80% identity to any one of SEQ ID NOs:89-139; and/or (c) encodes an amino acid sequence comprising a region having at least 80% identity to any one of SEQ ID NO:74, 77, 82, 85, or 88.

In some embodiments, an at least one mutation in an endogenous RPL3 gene in a plant may be a base substitution, a base deletion and/or a base insertion, optionally a non-natural mutation. In some embodiments, the at least one mutation in an endogenous RPL3 gene in a plant may result in a plant having the phenotype of (1) reduced binding of a trichothecene mycotoxin to the RPL3 polypeptide encoded by the mutated RPL3 gene(2) increased tolerance to deoxynivalenol (DON), (3) increased resistance/tolerance to fusarium head blight (FHB) (e.g., increased resistance to the FHB causal agent, Fusarium graminearum, and/or decreased FHB symptoms), (4) reduced Fusarium graminearum load, and/or (5) producing seed that accumulates less trichothecene mycotoxin (e.g., DON) when infected with Fusarium graminearum as compared to a plant devoid of the at least one mutation (e.g., an isogenic plant (e.g., wild type unedited plant or a null segregant).

In some embodiments, a mutation in an endogenous RPL3 gene may be a base substitution, a base deletion and/or a base insertion of at least 1 base pair. In some embodiments, a base deletion may be 1 nucleotide to about 100 consecutive nucleotides (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 base pairs, or any range or value therein, e.g., 1 to about 50 base pairs, 1 to about 30 base pairs, 1 to about 15 base pairs, or any range or value therein), optionally where the mutation is at about 2 to about 100 consecutive nucleotides (e.g., 2 to about 50, 60, 70, 80 or more consecutive base pairs, 2 to about 30 consecutive base pairs, 2 to about 15 consecutive base pairs)). In some embodiments, a mutation in an endogenous RPL3 gene may be a base insertion of 1 to about 16 nucleotides, optionally 1-16 consecutive nucleotides (e.g., 1, 2, 3, 4, 5, 6, 7, 8, 9, 10, 11, 12, 13, 14, 15 or 16 nucleotides) of the RPL3 nucleic acid. In some embodiments, a mutation in an endogenous RPL3 gene may be an out-of-frame insertion or an out-of-frame deletion that results in a truncated RPL3 protein (e.g., a truncated Ribosomal Protein L3 (RPL3) polypeptide), or little or no detectable RPL3 protein, and/or an RPL3 polypeptide that is non-functional in its ability to bind to trichothecene mycotoxins (e.g., DON), optionally wherein the mutation results in one or more of the phenotypes as described herein. In some embodiments, the at least one mutation may be a base substitution, optionally a substitution to an A, a T, a G, or a C. A mutation useful with this invention may be a point mutation. In some embodiments, the mutation may be a non-natural mutation.

In some embodiments, a mutation in an endogenous RPL3 gene may be made following cleavage by an editing system that comprises a nuclease and a nucleic acid binding domain that binds to a target site within a target nucleic acid (e.g., a RPL3 gene, e.g., e.g., a RPL3A gene, a RPL3A-1 gene, a RPL3A-2 gene, a RPL3A-3 gene, a RPL3B gene, a RPL3B-1 gene, a RPL3B-2 gene, and/or a RPL3B-3 gene), the target nucleic acid comprising a sequence having at least 80% sequence identity to any one of the nucleotide sequences of SEQ ID NO:72, 73, 75, 76, 78, 79, 80, 81, 83, 84, 86, or 87, and/or encoding an amino acid sequence having at least 80% sequence identity to any one of SEQ ID NO:74, 77, 82, 85, or 88, optionally wherein the target site is located in a region of the RPL3 gene: the region comprising a sequence having at least 80% identity to any one of SEQ ID NOs:89-139.

In some embodiments, a mutation as described herein may result in a mutated RPL3 gene having at least 90% sequence identity to any one of SEQ ID NOs:147, 149, 151, 153, or 155 and/or that encodes a mutated RPL3 polypeptide having at least 90% sequence identity to any one of SEQ ID NOs:148, 150, 152, 154, or 156. In some embodiments, a modified RPL3 polypeptide is provided that comprises a sequence having at least 90% sequence identity to any one of SEQ ID NOs:148, 150, 152, 154, or 156.

Further provided are guide nucleic acids (e.g., gRNA, gDNA, crRNA, crDNA) that bind to a target site in a Ribosomal Protein L3 (RPL3) gene (e.g., a RPL3A gene, a RPL3A-1 gene, a RPL3A-2 gene, a RPL3A-3 gene, a RPL3B gene, a RPL3B-1 gene, a RPL3B-2 gene, and/or a RPL3B-3 gene), wherein the target site is in a region of the RPL3 gene having at least 80% sequence identity to any one of the nucleotide sequences of SEQ ID NOs:89-139. In some embodiments, the guide nucleic acid comprises a spacer comprising any one or more of the nucleotide sequences of SEQ ID NOs:140-146, or a reverse complement thereof.

In some embodiments, a wheat plant or part thereof (Triticum aestivum) is provided that comprises at least one mutation in at least one endogenous Ribosomal Protein L3 (RPL3) gene having the gene identification number (gene ID) of TraesCS4A02G208000 (SEQ ID NO:72), TraesCS4B02G111500 (SEQ ID NO:75), TraesCS4D02G109200 (SEQ ID NO:78), TraesCS5A02G112400 (SEQ ID NO:80), TraesCS5B02G118400 (SEQ ID NO:83), and/or TraesCS5D02G129200 (SEQ ID NO:86), optionally wherein the mutation may be a non-natural mutation.

In some embodiments, a guide nucleic acid is provided that binds to a target nucleic acid in a Ribosomal Protein L3 (RPL3) gene, the RPL3 gene having the gene identification number (gene ID) of TraesCS4A02G208000 (SEQ ID NO:72), TraesCS4B02G111500 (SEQ ID NO:75), TraesCS4D02G109200 (SEQ ID NO:78), TraesCS5A02G112400 (SEQ ID NO:80), TraesCS5B02G118400 (SEQ ID NO:83), and/or TraesCS5D02G129200 (SEQ ID NO:86).

In some embodiments, a system is provided comprising a guide nucleic acid comprising a spacer (e.g., one or more spacers) having the nucleotide sequence of any one of SEQ ID NOs:140-146, or a reverse complement thereof, and a CRISPR-Cas effector protein that associates with the guide nucleic acid. In some embodiments, the system may further comprise a tracr nucleic acid that associates with the guide nucleic acid and a CRISPR-Cas effector protein, optionally wherein the tracr nucleic acid and the guide nucleic acid are covalently linked.

As used herein, “a CRISPR-Cas effector protein in association with a guide nucleic acid” refers to the complex that is formed between a CRISPR-Cas effector protein and a guide nucleic acid in order to direct the CRISPR-Cas effector protein to a target site in a gene.

The invention further provides a gene editing system comprising a CRISPR-Cas effector protein in association with a guide nucleic acid and the guide nucleic acid comprises a spacer sequence that binds to an endogenous RPL3 gene, optionally wherein the endogenous RPL3 (a) comprises a sequence having at least 80% sequence identity to any one of the nucleotide sequences of SEQ ID NO:72, 73, 75, 76, 78, 79, 80, 81, 83, 84, 86, or 87; (b) comprises a region having at least 80% identity to any one of SEQ ID NOs:89-139; and/or (c) encodes an amino acid sequence having at least 80% sequence identity to SEQ ID NO:74, 77, 82, 85, or 88. In some embodiments, a spacer sequence of the guide nucleic acid may comprise the nucleotide sequence of any of SEQ ID NOs:140-146, or a reverse complement thereof. In some embodiments, the gene editing system may further comprise a tracr nucleic acid that associates with the guide nucleic acid and a CRISPR-Cas effector protein, optionally wherein the tracr nucleic acid and the guide nucleic acid are covalently linked.

The present invention further provides a complex comprising a CRISPR-Cas effector protein comprising a cleavage domain and a guide nucleic acid, wherein the guide nucleic acid binds to a target site in an endogenous RPL3 gene, wherein the endogenous RPL3 gene: (a) comprises a sequence having at least 80% sequence identity to any one of the nucleotide sequences of SEQ ID NO:72, 73, 75, 76, 78, 79, 80, 81, 83, 84, 86, or 87; (b) comprises a region having at least 80% identity to any one of SEQ ID NOs:89-139; and/or (c) encodes an amino acid sequence having at least 80% sequence identity to SEQ ID NO:74, 77, 82, 85, or 88, and the cleavage domain cleaves a target strand in the RPL3 gene.

In some embodiments, an expression cassette(s) is/are provided that comprise (a) a polynucleotide encoding CRISPR-Cas effector protein comprising a cleavage domain and (b) a guide nucleic acid that binds to a target site in an endogenous RPL3 gene, wherein the guide nucleic acid comprises a spacer sequence that is complementary to and binds to (i) a portion of a nucleic acid having at least 80% sequence identity to any one of the nucleotide sequences of SEQ ID NO:72, 73, 75, 76, 78, 79, 80, 81, 83, 84, 86, or 87; (ii) a portion of a nucleic acid having at least 80% sequence identity to any one of the nucleotide sequences of SEQ ID NOs:89-139; and/or (iii) a portion of a nucleic acid encoding an amino acid sequence having at least 80% sequence identity to SEQ ID NO:74, 77, 82, 85, or 88.

Also provided are mutated nucleic acids encoding mutated Ribosomal Protein L3 (RPL3) polypeptides (e.g., RPL3A, RPL3A-1, RPL3A-2, RPL3A-3, RPL3B, RPL3B-1, RPL3B-2, and/or RPL3B-3), optionally wherein when present in a plant or plant part the mutated RPL3 polypeptide/mutated RPL3 gene results in the plant comprising a phenotype of a phenotype of (1) reduced binding of a trichothecene mycotoxin to the RPL3 polypeptide encoded by a modified RPL3 gene, (2) increased tolerance to deoxynivalenol (DON), (3) increased resistance/tolerance to fusarium head blight (FHB) (e.g., increased resistance to the FHB causal agent, Fusarium graminearum, and/or decreased FHB symptoms), (4) reduced Fusarium graminearum load, and/or (5) producing seed that accumulates less trichothecene mycotoxin (e.g., DON) when infected with Fusarium graminearum as compared to a plant devoid of the at least one mutation (e.g., an isogenic plant (e.g., wild type unedited plant or a null segregant).

Nucleic acid constructs of the invention (e.g., a construct comprising a sequence specific nucleic acid binding domain (e.g., sequence specific DNA binding domain), a CRISPR-Cas effector domain, a deaminase domain, reverse transcriptase (RT), RT template and/or a guide nucleic acid, etc.) and expression cassettes/vectors comprising the same may be used as an editing system of this invention for modifying target nucleic acids (e.g., endogenous RPL3 genes, e.g., endogenous RPL3A gene, endogenous RPL3A-1 gene, endogenous RPL3A-2 gene, endogenous RPL3A-3gene, endogenous RPL3B gene, endogenous RPL3B-1 gene, endogenous RPL3B-2 gene, endogenous RPL3B-3gene) and/or their expression.

Any plant comprising an endogenous RPL3 gene that is capable of conferring at least one of the phenotypes of (1) reduced binding of a trichothecene mycotoxin to the RPL3 polypeptide encoded by a modified RPL3 gene, (2) increased tolerance to deoxynivalenol (DON), (3) increased resistance/tolerance to fusarium head blight (FHB) (e.g., increased resistance to the FHB causal agent, Fusarium graminearum, and/or decreased FHB symptoms), (4) reduced Fusarium graminearum load, and/or (5) producing seed that accumulates less trichothecene mycotoxin (e.g., DON) when modified as described herein (and when infected with Fusarium graminearum) as compared to a plant devoid of the at least one mutation (e.g., an isogenic plant (e.g., wild type unedited plant or a null segregant). Such endogenous RPL3 gene may be modified (e.g., mutated, e.g., base edited, cleaved, nicked, etc.) as described herein (e.g., using the polypeptides, polynucleotides, RNPs, nucleic acid constructs, expression cassettes, and/or vectors of the invention) to improve one or more yield traits in the plant.

An editing system useful with this invention can be any site-specific (sequence-specific) genome editing system now known or later developed, which system can introduce mutations in a target specific manner. For example, an editing system (e.g., site- or sequence-specific editing system) can include, but is not limited to, a CRISPR-Cas editing system, a meganuclease editing system, a zinc finger nuclease (ZFN) editing system, a transcription activator-like effector nuclease (TALEN) editing system, a base editing system and/or a prime editing system, each of which can comprise one or more polypeptides and/or one or more polynucleotides that when expressed as a system in a cell can modify (mutate) a target nucleic acid in a sequence specific manner. In some embodiments, an editing system (e.g., site- or sequence-specific editing system) can comprise one or more polynucleotides and/or one or more polypeptides, including but not limited to a nucleic acid binding domain (DNA binding domain), a nuclease, and/or other polypeptide, and/or a polynucleotide.

In some embodiments, an editing system can comprise one or more sequence-specific nucleic acid binding domains (DNA binding domains) that can be from, for example, a polynucleotide-guided endonuclease, a CRISPR-Cas endonuclease (e.g., CRISPR-Cas effector protein), a zinc finger nuclease, a transcription activator-like effector nuclease (TALEN) and/or an Argonaute protein. In some embodiments, an editing system can comprise one or more cleavage domains (e.g., nucleases) including, but not limited to, an endonuclease (e.g., Fok1), a polynucleotide-guided endonuclease, a CRISPR-Cas endonuclease (e.g., CRISPR-Cas effector protein), a zinc finger nuclease, and/or a transcription activator-like effector nuclease (TALEN). In some embodiments, an editing system can comprise one or more polypeptides that include, but are not limited to, a deaminase (e.g., a cytosine deaminase, an adenine deaminase), a reverse transcriptase, a Dna2 polypeptide, and/or a 5′ flap endonuclease (FEN). In some embodiments, an editing system can comprise one or more polynucleotides, including, but is not limited to, a CRISPR array (CRISPR guide) nucleic acid, extended guide nucleic acid, and/or a reverse transcriptase template.

In some embodiments, a method of modifying or editing Ribosomal Protein L3 (RPL3) gene may comprise contacting a target nucleic acid (e.g., a nucleic acid encoding a Ribosomal Protein L3 (RPL3) polypeptide, e.g., a RPL3A polypeptide, a RPL3B polypeptide, and the like) with a base-editing fusion protein (e.g., a sequence specific DNA binding protein (e.g., a CRISPR-Cas effector protein or domain) fused to a deaminase domain (e.g., an adenine deaminase and/or a cytosine deaminase) and a guide nucleic acid, wherein the guide nucleic acid is capable of guiding/targeting the base editing fusion protein to the target nucleic acid, thereby editing a locus within the target nucleic acid. In some embodiments, a base editing fusion protein and guide nucleic acid may be comprised in one or more expression cassettes. In some embodiments, the target nucleic acid may be contacted with a base editing fusion protein and an expression cassette comprising a guide nucleic acid. In some embodiments, the sequence-specific nucleic acid binding fusion proteins and guides may be provided as ribonucleoproteins (RNPs). In some embodiments, a cell may be contacted with more than one base-editing fusion protein and/or one or more guide nucleic acids that may target one or more target nucleic acids in the cell.

In some embodiments, a method of modifying or editing a Ribosomal Protein L3 (RPL3) gene may comprise contacting a target nucleic acid (e.g., a nucleic acid encoding a RPL3 polypeptide) with a sequence-specific nucleic acid binding fusion protein (e.g., a sequence-specific DNA binding protein (e.g., a CRISPR-Cas effector protein or domain) fused to a peptide tag, a deaminase fusion protein comprising a deaminase domain (e.g., an adenine deaminase and/or a cytosine deaminase) fused to an affinity polypeptide that is capable of binding to the peptide tag, and a guide nucleic acid, wherein the guide nucleic acid is capable of guiding/targeting the sequence-specific nucleic acid binding fusion protein to the target nucleic acid and the sequence-specific nucleic acid binding fusion protein is capable of recruiting the deaminase fusion protein to the target nucleic acid via the peptide tag-affinity polypeptide interaction, thereby editing a locus within the target nucleic acid. In some embodiments, the sequence-specific nucleic acid binding fusion protein may be fused to the affinity polypeptide that binds the peptide tag and the deaminase may be fused to the peptide tag, thereby recruiting the deaminase to the sequence-specific nucleic acid binding fusion protein and to the target nucleic acid. In some embodiments, the sequence-specific binding fusion protein, deaminase fusion protein, and guide nucleic acid may be comprised in one or more expression cassettes. In some embodiments, the target nucleic acid may be contacted with a sequence-specific binding fusion protein, deaminase fusion protein, and an expression cassette comprising a guide nucleic acid. In some embodiments, the sequence-specific nucleic acid binding fusion proteins, deaminase fusion proteins and guides may be provided as ribonucleoproteins (RNPs).

In some embodiments, methods such as prime editing may be used to generate a mutation in an endogenous RPL3 gene. In prime editing, RNA-dependent DNA polymerase (reverse transcriptase, RT) and reverse transcriptase templates (RT template) are used in combination with sequence specific nucleic acid binding domains that confer the ability to recognize and bind the target in a sequence-specific manner, and which can also cause a nick of the PAM-containing strand within the target. The nucleic acid binding domain may be a CRISPR-Cas effector protein and in this case, the CRISPR array or guide RNA may be an extended guide that comprises an extended portion comprising a primer binding site (PSB) and the edit to be incorporated into the genome (the template). Similar to base editing, prime editing can take advantages of the various methods of recruiting proteins for use in the editing to the target site, such methods including both non-covalent and covalent interactions between the proteins and nucleic acids used in the selected process of genome editing.

As used herein, a “CRISPR-Cas effector protein” is a protein or polypeptide or domain thereof that cleaves or cuts a nucleic acid, binds a nucleic acid (e.g., a target nucleic acid and/or a guide nucleic acid), and/or that identifies, recognizes, or binds a guide nucleic acid as defined herein. In some embodiments, a CRISPR-Cas effector protein may be an enzyme (e.g., a nuclease, endonuclease, nickase, etc.) or portion thereof and/or may function as an enzyme. In some embodiments, a CRISPR-Cas effector protein refers to a CRISPR-Cas nuclease polypeptide or domain thereof that comprises nuclease activity or in which the nuclease activity has been reduced or eliminated, and/or comprises nickase activity or in which the nickase has been reduced or eliminated, and/or comprises single stranded DNA cleavage activity (ss DNAse activity) or in which the ss DNAse activity has been reduced or eliminated, and/or comprises self-processing RNAse activity or in which the self-processing RNAse activity has been reduced or eliminated. A CRISPR-Cas effector protein may bind to a target nucleic acid.

In some embodiments, a sequence-specific nucleic acid binding domain may be a CRISPR-Cas effector protein. In some embodiments, a CRISPR-Cas effector protein may be from a Type I CRISPR-Cas system, a Type II CRISPR-Cas system, a Type III CRISPR-Cas system, a Type IV CRISPR-Cas system, Type V CRISPR-Cas system, or a Type VI CRISPR-Cas system. In some embodiments, a CRISPR-Cas effector protein of the invention may be from a Type II CRISPR-Cas system or a Type V CRISPR-Cas system. In some embodiments, a CRISPR-Cas effector protein may be Type II CRISPR-Cas effector protein, for example, a Cas9 effector protein. In some embodiments, a CRISPR-Cas effector protein may be Type V CRISPR-Cas effector protein, for example, a Cas12 effector protein.

In some embodiments, a CRISPR-Cas effector protein may include, but is not limited to, a Cas9, C2c1, C2c3, Cas12a (also referred to as Cpf1), Cas12b, Cas12c, Cas12d, Cas12e, Cas13a, Cas13b, Cas13c, Cas13d, Casl, CaslB, Cas2, Cas3, Cas3′, Cas3″, Cas4, Cas5, Cas6, Cas7, Cas8, Cas9 (also known as Csnl and Csx12), Cas10, Csyl, Csy2, Csy3, Csel, Cse2, Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csxl7, Csxl4, Csx10, Csx16, CsaX, Csx3, Csxl, Csxl5, Csfl, Csf2, Csf3, Csf4 (dinG), and/or Csf5 nuclease, optionally wherein the CRISPR-Cas effector protein may be a Cas9, Cas12a (Cpf1), Cas12b, Cas12c (C2c3), Cas12d (CasY), Cas12e (CasX), Cas12g, Cas12h, Cas12i, C2c4, C2c5, C2c8, C2c9, C2c10, Cas14a, Cas14b, and/or Cas14c effector protein.

In some embodiments, a CRISPR-Cas effector protein useful with the invention may comprise a mutation in its nuclease active site (e.g., RuvC, HNH, e.g., RuvC site of a Cas12a nuclease domain; e.g., RuvC site and/or HNH site of a Cas9 nuclease domain). A CRISPR-Cas effector protein having a mutation in its nuclease active site, and therefore, no longer comprising nuclease activity, is commonly referred to as “dead,” e.g., dCas. In some embodiments, a CRISPR-Cas effector protein domain or polypeptide having a mutation in its nuclease active site may have impaired activity or reduced activity as compared to the same CRISPR-Cas effector protein without the mutation, e.g., a nickase, e.g., Cas9 nickase, Cas12anickase.

A CRISPR Cas9 effector protein or CRISPR Cas9 effector domain useful with this invention may be any known or later identified Cas9 nuclease. In some embodiments, a CRISPR Cas9 polypeptide can be a Cas9 polypeptide from, for example, Streptococcus spp. (e.g., S. pyogenes, S. thermophilus), Lactobacillus spp., Bifidobacterium spp., Kandleria spp., Leuconostoc spp., Oenococcus spp., Pediococcus spp., Weissella spp., and/or Olsenella spp. Example Cas9 sequences include, but are not limited to, the amino acid sequences of SEQ ID NO:59 and SEQ ID NO:60 or the nucleotide sequences of SEQ ID NOs:61-71.

In some embodiments, the CRISPR-Cas effector protein may be a Cas9 polypeptide derived from Streptococcus pyogenes and recognizes the PAM sequence motif NGG, NAG, NGA (Mali et al, Science 2013; 339(6121): 823-826). In some embodiments, the CRISPR-Cas effector protein may be a Cas9 polypeptide derived from Streptococcus thermophiles and recognizes the PAM sequence motif NGGNG and/or NNAGAAW (W = A or T) (See, e.g., Horvath et al, Science, 2010; 327(5962): 167-170, and Deveau et al, J Bacteriol 2008; 190(4): 1390-1400). In some embodiments, the CRISPR-Cas effector protein may be a Cas9 polypeptide derived from Streptococcus mutans and recognizes the PAM sequence motif NGG and/or NAAR (R = A or G) (See, e.g., Deveau et al, J BACTERIOL 2008; 190(4): 1390-1400). In some embodiments, the CRISPR-Cas effector protein may be a Cas9 polypeptide derived from Streptococcus aureus and recognizes the PAM sequence motif NNGRR (R = A or G). In some embodiments, the CRISPR-Cas effector protein may be a Cas9 protein derived from S. aureus, which recognizes the PAM sequence motif N GRRT (R = A or G). In some embodiments, the CRISPR-Cas effector protein may be a Cas9 polypeptide derived from S. aureus, which recognizes the PAM sequence motif N GRRV (R = A or G). In some embodiments, the CRISPR-Cas effector protein may be a Cas9 polypeptide that is derived from Neisseria meningitidis and recognizes the PAM sequence motif N GATT or N GCTT (R = A or G, V = A, G or C) (See, e.g., Hou et ah, PNAS 2013, 1-6). In the aforementioned embodiments, N can be any nucleotide residue, e.g., any of A, G, C or T. In some embodiments, the CRISPR-Cas effector protein may be a Cas13a protein derived from Leptotrichia shahii, which recognizes a protospacer flanking sequence (PFS) (or RNA PAM (rPAM)) sequence motif of a single 3′ A, U, or C, which may be located within the target nucleic acid.

In some embodiments, the CRISPR-Cas effector protein may be derived from Cas12a, which is a Type V Clustered Regularly Interspaced Short Palindromic Repeats (CRISPR)-Cas nuclease see, e.g., SEQ ID NOs:1-20). Cas12a differs in several respects from the more well-known Type II CRISPR Cas9 nuclease. For example, Cas9 recognizes a G-rich protospacer-adjacent motif (PAM) that is 3′ to its guide RNA (gRNA, sgRNA, crRNA, crDNA, CRISPR array) binding site (protospacer, target nucleic acid, target DNA) (3′-NGG), while Cas12a recognizes a T-rich PAM that is located 5′ to the target nucleic acid (5′-TTN, 5′-TTTN. In fact, the orientations in which Cas9 and Cas12abind their guide RNAs are very nearly reversed in relation to their N and C termini. Furthermore, Cas12aenzymes use a single guide RNA (gRNA, CRISPR array, crRNA) rather than the dual guide RNA (sgRNA (e.g., crRNA and tracrRNA)) found in natural Cas9 systems, and Cas12a processes its own gRNAs. Additionally, Cas12a nuclease activity produces staggered DNA double stranded breaks instead of blunt ends produced by Cas9 nuclease activity, and Cas12a relies on a single RuvC domain to cleave both DNA strands, whereas Cas9 utilizes an HNH domain and a RuvC domain for cleavage.

A CRISPR Cas12a effector protein/domain useful with this invention may be any known or later identified Cas12apolypeptide (previously known as Cpf1) (see, e.g., U.S. Pat. No. 9,790,490, which is incorporated by reference for its disclosures of Cpf1 (Cas12a) sequences). The term “Cas12a”, “Cas12a polypeptide” or “Cas12a domain” refers to an RNA-guided nuclease comprising a Cas12apolypeptide, or a fragment thereof, which comprises the guide nucleic acid binding domain of Cas12a and/or an active, inactive, or partially active DNA cleavage domain of Cas 12a. In some embodiments, a Cas12a useful with the invention may comprise a mutation in the nuclease active site (e.g., RuvC site of the Cas12adomain). A Cas12a domain or Cas12a polypeptide having a mutation in its nuclease active site, and therefore, no longer comprising nuclease activity, is commonly referred to as deadCas12a (e.g., dCas12a). In some embodiments, a Cas12a domain or Cas12a polypeptide having a mutation in its nuclease active site may have impaired activity, e.g., may have nickase activity.

Any deaminase domain/polypeptide useful for base editing may be used with this invention. In some embodiments, the deaminase domain may be a cytosine deaminase domain or an adenine deaminase domain. A cytosine deaminase (or cytidine deaminase) useful with this invention may be any known or later identified cytosine deaminase from any organism (see, e.g., U.S. Pat. No. 10,167,457 and Thuronyi et al. Nat. Biotechnol. 37:1070-1079 (2019), each of which is incorporated by reference herein for its disclosure of cytosine deaminases). Cytosine deaminases can catalyze the hydrolytic deamination of cytidine or deoxycytidine to uridine or deoxyuridine, respectively. Thus, in some embodiments, a deaminase or deaminase domain useful with this invention may be a cytidine deaminase domain, catalyzing the hydrolytic deamination of cytosine to uracil. In some embodiments, a cytosine deaminase may be a variant of a naturally occurring cytosine deaminase, including but not limited to a primate (e.g., a human, monkey, chimpanzee, gorilla), a dog, a cow, a rat or a mouse. Thus, in some embodiments, a cytosine deaminase useful with the invention may be about 70% to about 100% identical to a wild type cytosine deaminase (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, and any range or value therein, to a naturally occurring cytosine deaminase).

In some embodiments, a cytosine deaminase useful with the invention may be an apolipoprotein B mRNA-editing complex (APOBEC) family deaminase. In some embodiments, the cytosine deaminase may be an APOBEC1 deaminase, an APOBEC2 deaminase, an APOBEC3A deaminase, an APOBEC3B deaminase, an APOBEC3C deaminase, an APOBEC3D deaminase, an APOBEC3F deaminase, an APOBEC3G deaminase, an APOBEC3H deaminase, an APOBEC4 deaminase, a human activation induced deaminase (hAID), an rAPOBEC1, FERNY, and/or a CDA1, optionally a pmCDA1, an atCDA1 (e.g., At2g19570), and evolved versions of the same (e.g., SEQ ID NO:27, SEQ ID NO:28 or SEQ ID NO:29). In some embodiments, the cytosine deaminase may be an APOBEC1 deaminase having the amino acid sequence of SEQ ID NO:23. In some embodiments, the cytosine deaminase may be an APOBEC3A deaminase having the amino acid sequence of SEQ ID NO:24. In some embodiments, the cytosine deaminase may be an CDA1 deaminase, optionally a CDA1 having the amino acid sequence of SEQ ID NO:25. In some embodiments, the cytosine deaminase may be a FERNY deaminase, optionally a FERNY having the amino acid sequence of SEQ ID NO:26. In some embodiments, a cytosine deaminase useful with the invention may be about 70% to about 100% identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% identical) to the amino acid sequence of a naturally occurring cytosine deaminase (e.g., an evolved deaminase). In some embodiments, a cytosine deaminase useful with the invention may be about 70% to about 99.5% identical (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.5% identical) to the amino acid sequence of SEQ ID NO:23, SEQ ID NO:24, SEQ ID NO:25 or SEQ ID NO:26 (e.g., at least 80%, at least 85%, at least 90%, at least 92%, at least 95%, at least 96%, at least 97%, at least 98%, at least 99%, or at least 99.5% identical to the amino acid sequence of SEQ ID NO:23, SEQ ID NO:24, SEQ ID NO:25, SEQ ID NO:26, SEQ ID NO:27, SEQ ID NO:28 or SEQ ID NO:29). In some embodiments, a polynucleotide encoding a cytosine deaminase may be codon optimized for expression in a plant and the codon optimized polypeptide may be about 70% to 99.5% identical to the reference polynucleotide.

In some embodiments, a nucleic acid construct of this invention may further encode a uracil glycosylase inhibitor (UGI) (e.g., uracil-DNA glycosylase inhibitor) polypeptide/domain. Thus, in some embodiments, a nucleic acid construct encoding a CRISPR-Cas effector protein and a cytosine deaminase domain (e.g., encoding a fusion protein comprising a CRISPR-Cas effector protein domain fused to a cytosine deaminase domain, and/or a CRISPR-Cas effector protein domain fused to a peptide tag or to an affinity polypeptide capable of binding a peptide tag and/or a deaminase protein domain fused to a peptide tag or to an affinity polypeptide capable of binding a peptide tag) may further encode a uracil-DNA glycosylase inhibitor (UGI), optionally wherein the UGI may be codon optimized for expression in a plant. In some embodiments, the invention provides fusion proteins comprising a CRISPR-Cas effector polypeptide, a deaminase domain, and a UGI and/or one or more polynucleotides encoding the same, optionally wherein the one or more polynucleotides may be codon optimized for expression in a plant. In some embodiments, the invention provides fusion proteins, wherein a CRISPR-Cas effector polypeptide, a deaminase domain, and a UGI may be fused to any combination of peptide tags and affinity polypeptides as described herein, thereby recruiting the deaminase domain and UGI to the CRISPR-Cas effector polypeptide and a target nucleic acid. In some embodiments, a guide nucleic acid may be linked to a recruiting RNA motif and one or more of the deaminase domain and/or UGI may be fused to an affinity polypeptide that is capable of interacting with the recruiting RNA motif, thereby recruiting the deaminase domain and UGI to a target nucleic acid.

A “uracil glycosylase inhibitor” useful with the invention may be any protein that is capable of inhibiting a uracil-DNA glycosylase base-excision repair enzyme. In some embodiments, a UGI domain comprises a wild type UGI or a fragment thereof. In some embodiments, a UGI domain useful with the invention may be about 70% to about 100% identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% identical and any range or value therein) to the amino acid sequence of a naturally occurring UGI domain. In some embodiments, a UGI domain may comprise the amino acid sequence of SEQ ID NO:41 or a polypeptide having about 70% to about 99.5% sequence identity to the amino acid sequence of SEQ ID NO:41 (e.g., at least 80%, at least 85%, at least 90%, at least 92%, at least 95%, at least 96%, at least 97%, at least 98%, at least 99%, or at least 99.5% identical to the amino acid sequence of SEQ ID NO:41). For example, in some embodiments, a UGI domain may comprise a fragment of the amino acid sequence of SEQ ID NO:41 that is 100% identical to a portion of consecutive nucleotides (e.g., 10, 15, 20, 25, 30, 35, 40, 45, 50, 55, 60, 65, 70, 75, 80 consecutive nucleotides; e.g., about 10, 15, 20, 25, 30, 35, 40, 45, to about 50, 55, 60, 65, 70, 75, 80 consecutive nucleotides) of the amino acid sequence of SEQ ID NO:41. In some embodiments, a UGI domain may be a variant of a known UGI (e.g., SEQ ID NO:41) having about 70% to about 99.5% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% sequence identity, and any range or value therein) to the known UGI. In some embodiments, a polynucleotide encoding a UGI may be codon optimized for expression in a plant (e.g., a plant) and the codon optimized polypeptide may be about 70% to about 99.5% identical to the reference polynucleotide.

An adenine deaminase (or adenosine deaminase) useful with this invention may be any known or later identified adenine deaminase from any organism (see, e.g., U.S. Pat. No. 10,113,163, which is incorporated by reference herein for its disclosure of adenine deaminases). An adenine deaminase can catalyze the hydrolytic deamination of adenine or adenosine. In some embodiments, the adenine deaminase may catalyze the hydrolytic deamination of adenosine or deoxyadenosine to inosine or deoxyinosine, respectively. In some embodiments, the adenosine deaminase may catalyze the hydrolytic deamination of adenine or adenosine in DNA. In some embodiments, an adenine deaminase encoded by a nucleic acid construct of the invention may generate an A→G conversion in the sense (e.g., “+”; template) strand of the target nucleic acid or a T→C conversion in the antisense (e.g., “-”, complementary) strand of the target nucleic acid.

In some embodiments, an adenosine deaminase may be a variant of a naturally occurring adenine deaminase. Thus, in some embodiments, an adenosine deaminase may be about 70% to 100% identical to a wild type adenine deaminase (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, and any range or value therein, to a naturally occurring adenine deaminase). In some embodiments, the deaminase or deaminase does not occur in nature and may be referred to as an engineered, mutated or evolved adenosine deaminase. Thus, for example, an engineered, mutated or evolved adenine deaminase polypeptide or an adenine deaminase domain may be about 70% to 99.9% identical to a naturally occurring adenine deaminase polypeptide/domain (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% identical, and any range or value therein, to a naturally occurring adenine deaminase polypeptide or adenine deaminase domain). In some embodiments, the adenosine deaminase may be from a bacterium, (e.g., Escherichia coli, Staphylococcus aureus, Haemophilus influenzae, Caulobacter crescentus, and the like). In some embodiments, a polynucleotide encoding an adenine deaminase polypeptide/domain may be codon optimized for expression in a plant.

In some embodiments, an adenine deaminase domain may be a wild type tRNA-specific adenosine deaminase domain, e.g., a tRNA-specific adenosine deaminase (TadA) and/or a mutated/evolved adenosine deaminase domain, e.g., mutated/evolved tRNA-specific adenosine deaminase domain (TadA*). In some embodiments, a TadA domain may be from E. coli. In some embodiments, the TadA may be modified, e.g., truncated, missing one or more N-terminal and/or C-terminal amino acids relative to a full-length TadA (e.g., 1, 2, 3, 4, 5, 6, 7, 8, 9, 10, 11, 12, 13, 14, 15, 6, 17, 18, 19, or 20 N-terminal and/or C terminal amino acid residues may be missing relative to a full length TadA. In some embodiments, a TadA polypeptide or TadA domain does not comprise an N-terminal methionine. In some embodiments, a wild type E. coli TadA comprises the amino acid sequence of SEQ ID NO:30. In some embodiments, a mutated/evolved E. coli TadA* comprises the amino acid sequence of SEQ ID NOs:31-40 (e.g., SEQ ID NOs: 31, 32, 33, 34, 35, 36, 37, 38, 39 or 40). In some embodiments, a polynucleotide encoding a TadA/TadA* may be codon optimized for expression in a plant.

A cytosine deaminase catalyzes cytosine deamination and results in a thymidine (through a uracil intermediate), causing a C to T conversion, or a G to A conversion in the complementary strand in the genome. Thus, in some embodiments, the cytosine deaminase encoded by the polynucleotide of the invention generates a C→T conversion in the sense (e.g., “+”; template) strand of the target nucleic acid or a G →A conversion in antisense (e.g., “-”, complementary) strand of the target nucleic acid.

In some embodiments, the adenine deaminase encoded by the nucleic acid construct of the invention generates an A→G conversion in the sense (e.g., “+”; template) strand of the target nucleic acid or a T→C conversion in the antisense (e.g., “-”, complementary) strand of the target nucleic acid.

The nucleic acid constructs of the invention encoding a base editor comprising a sequence-specific nucleic acid binding protein and a cytosine deaminase polypeptide, and nucleic acid constructs/expression cassettes/vectors encoding the same, may be used in combination with guide nucleic acids for modifying target nucleic acid including, but not limited to, generation of C→T or G →A mutations in a target nucleic acid including, but not limited to, a plasmid sequence; generation of C→T or G →A mutations in a coding sequence to alter an amino acid identity; generation of C→T or G →A mutations in a coding sequence to generate a stop codon; generation of C→T or G →A mutations in a coding sequence to disrupt a start codon; generation of point mutations in genomic DNA to disrupt function; and/or generation of point mutations in genomic DNA to disrupt splice junctions.

The nucleic acid constructs of the invention encoding a base editor comprising a sequence-specific nucleic acid binding protein and an adenine deaminase polypeptide, and expression cassettes and/or vectors encoding the same may be used in combination with guide nucleic acids for modifying a target nucleic acid including, but not limited to, generation of A→G or T→C mutations in a target nucleic acid including, but not limited to, a plasmid sequence; generation of A→G or T→C mutations in a coding sequence to alter an amino acid identity; generation of A→G or T→C mutations in a coding sequence to generate a stop codon; generation of A→G or T→C mutations in a coding sequence to disrupt a start codon; generation of point mutations in genomic DNA to disrupt function; and/or generation of point mutations in genomic DNA to disrupt splice junctions.

The nucleic acid constructs of the invention comprising a CRISPR-Cas effector protein or a fusion protein thereof may be used in combination with a guide RNA (gRNA, CRISPR array, CRISPR RNA, crRNA), designed to function with the encoded CRISPR-Cas effector protein or domain, to modify a target nucleic acid. A guide nucleic acid useful with this invention comprises at least one spacer sequence and at least one repeat sequence. The guide nucleic acid is capable of forming a complex with the CRISPR-Cas nuclease domain encoded and expressed by a nucleic acid construct of the invention and the spacer sequence is capable of hybridizing to a target nucleic acid, thereby guiding the complex (e.g., a CRISPR-Cas effector fusion protein (e.g., CRISPR-Cas effector domain fused to a deaminase domain and/or a CRISPR-Cas effector domain fused to a peptide tag or an affinity polypeptide to recruit a deaminase domain and optionally, a UGI) to the target nucleic acid, wherein the target nucleic acid may be modified (e.g., cleaved or edited) or modulated (e.g., modulating transcription) by the deaminase domain.

As an example, a nucleic acid construct encoding a Cas9 domain linked to a cytosine deaminase domain (e.g., fusion protein) may be used in combination with a Cas9 guide nucleic acid to modify a target nucleic acid, wherein the cytosine deaminase domain of the fusion protein deaminates a cytosine base in the target nucleic acid, thereby editing the target nucleic acid. In a further example, a nucleic acid construct encoding a Cas9 domain linked to an adenine deaminase domain (e.g., fusion protein) may be used in combination with a Cas9 guide nucleic acid to modify a target nucleic acid, wherein the adenine deaminase domain of the fusion protein deaminates an adenosine base in the target nucleic acid, thereby editing the target nucleic acid.

Likewise, a nucleic acid construct encoding a Cas12a domain (or other selected CRISPR-Cas nuclease, e.g., C2c1, C2c3, Cas12b, Cas12c, Cas12d, Cas12e, Cas13a, Cas13b, Cas13c, Cas13d, Casl, CaslB, Cas2, Cas3, Cas3′, Cas3″, Cas4, Cas5, Cas6, Cas7, Cas8, Cas9 (also known as Csnl and Csx12), Cas10, Csyl, Csy2, Csy3, Csel, Cse2, Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csx17, Csxl4, Csx10, Csx16, CsaX, Csx3, Csxl, Csxl5, Csfl, Csf2, Csf3, Csf4 (dinG), and/or Csf5) linked to a cytosine deaminase domain or adenine deaminase domain (e.g., fusion protein) may be used in combination with a Cas12aguide nucleic acid (or the guide nucleic acid for the other selected CRISPR-Cas nuclease) to modify a target nucleic acid, wherein the cytosine deaminase domain or adenine deaminase domain of the fusion protein deaminates a cytosine base in the target nucleic acid, thereby editing the target nucleic acid.

A “guide nucleic acid,” “guide RNA,” “gRNA,” “CRISPR RNA/DNA” “crRNA” or “crDNA” as used herein means a nucleic acid that comprises at least one spacer sequence, which is complementary to (and hybridizes to) a target DNA (e.g., protospacer), and at least one repeat sequence (e.g., a repeat of a Type V Cas12aCRISPR-Cas system, or a fragment or portion thereof; a repeat of a Type II Cas9 CRISPR-Cas system, or fragment thereof; a repeat of a Type V C2c1 CRISPR Cas system, or a fragment thereof; a repeat of a CRISPR-Cas system of, for example, C2c3, Cas12a (also referred to as Cpf1), Cas12b, Cas12c, Cas12d, Cas12e, Cas13a, Cas13b, Cas13c, Cas13d, Casl, CaslB, Cas2, Cas3, Cas3′, Cas3″, Cas4, Cas5, Cas6, Cas7, Cas8, Cas9 (also known as Csnl and Csx12), Cas10, Csyl, Csy2, Csy3, Csel, Cse2, Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csx17, Csxl4, Csx10, Csx16, CsaX, Csx3, Csxl, Csxl5, Csfl, Csf2, Csf3, Csf4 (dinG), and/or Csf5, or a fragment thereof), wherein the repeat sequence may be linked to the 5′ end and/or the 3′ end of the spacer sequence. The design of a gRNA of this invention may be based on a Type I, Type II, Type III, Type IV, Type V, or Type VI CRISPR-Cas system.

In some embodiments, a Cas12a gRNA may comprise, from 5′ to 3′, a repeat sequence (full length or portion thereof (“handle”); e.g., pseudoknot-like structure) and a spacer sequence.

In some embodiments, a guide nucleic acid may comprise more than one repeat sequence-spacer sequence (e.g., 2, 3, 4, 5, 6, 7, 8, 9, 10, or more repeat-spacer sequences) (e.g., repeat-spacer-repeat, e.g., repeat-spacer-repeat-spacer-repeat-spacer-repeat-spacer-repeat-spacer, and the like). The guide nucleic acids of this invention are synthetic, human-made, and not found in nature. A gRNA can be quite long and may be used as an aptamer (like in the MS2 recruitment strategy) or other RNA structures hanging off the spacer.

A “repeat sequence” as used herein, refers to, for example, any repeat sequence of a wild-type CRISPR Cas locus (e.g., a Cas9 locus, a Cas12a locus, a C2c1 locus, etc.) or a repeat sequence of a synthetic crRNA that is functional with the CRISPR-Cas effector protein encoded by the nucleic acid constructs of the invention. A repeat sequence useful with this invention can be any known or later identified repeat sequence of a CRISPR-Cas locus (e.g., Type I, Type II, Type III, Type IV, Type V or Type VI) or it can be a synthetic repeat designed to function in a Type I, II, III, IV, V or VI CRISPR-Cas system. A repeat sequence may comprise a hairpin structure and/or a stem loop structure. In some embodiments, a repeat sequence may form a pseudoknot-like structure at its 5′ end (i.e., “handle”). Thus, in some embodiments, a repeat sequence can be identical to or substantially identical to a repeat sequence from wild-type Type I CRISPR-Cas loci, Type II, CRISPR-Cas loci, Type III, CRISPR-Cas loci, Type IV CRISPR-Cas loci, Type V CRISPR-Cas loci and/or Type VI CRISPR-Cas loci. A repeat sequence from a wild-type CRISPR-Cas locus may be determined through established algorithms, such as using the CRISPRfinder offered through CRISPRdb (see, Grissa et al. Nucleic Acids Res. 35(Web Server issue):W52-7). In some embodiments, a repeat sequence or portion thereof is linked at its 3′ end to the 5′ end of a spacer sequence, thereby forming a repeat-spacer sequence (e.g., guide nucleic acid, guide RNA/DNA, crRNA, crDNA).

In some embodiments, a repeat sequence comprises, consists essentially of, or consists of at least 10 nucleotides depending on the particular repeat and whether the guide nucleic acid comprising the repeat is processed or unprocessed (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 to 100 or more nucleotides, or any range or value therein). In some embodiments, a repeat sequence comprises, consists essentially of, or consists of about 10 to about 20, about 10 to about 30, about 10 to about 45, about 10 to about 50, about 15 to about 30, about 15 to about 40, about 15 to about 45, about 15 to about 50, about 20 to about 30, about 20 to about 40, about 20 to about 50, about 30 to about 40, about 40 to about 80, about 50 to about 100 or more nucleotides.

A repeat sequence linked to the 5′ end of a spacer sequence can comprise a portion of a repeat sequence (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more contiguous nucleotides of a wild type repeat sequence). In some embodiments, a portion of a repeat sequence linked to the 5′ end of a spacer sequence can be about five to about ten consecutive nucleotides in length (e.g., about 5, 6, 7, 8, 9, 10 nucleotides) and have at least 90% sequence identity (e.g., at least about 90%, 91%, 92%, 93%, 94%, 95%, 96%, 97%, 98%, 99%, or more (e.g., 99.1, 99.2, 99.3, 99.4, 99.5, 99.6, 99.7, 99.8, 99.9, or 100%)) to the same region (e.g., 5′ end) of a wild type CRISPR Cas repeat nucleotide sequence. In some embodiments, a portion of a repeat sequence may comprise a pseudoknot-like structure at its 5′ end (e.g., “handle”).

A “spacer sequence” as used herein is a nucleotide sequence that is complementary to a target nucleic acid (e.g., target DNA) (e.g., protospacer) (e.g., a portion of consecutive nucleotides of a sequence (a) having at least 80% sequence identity to a nucleotide sequence of any one of SEQ ID NO:72, 73, 75, 76, 78, 79, 80, 81, 83, 84, 86, or 87, or SEQ ID NOs:89-139, and/or (b) encoding an amino acid sequence having at least 80% sequence identity to any one of SEQ ID NO:74, 77, 82, 85, or 88). In some embodiments, a spacer sequence (e.g., one or more spacers) may include, but is not limited to, the nucleotide sequences of any one of SEQ ID NOs:140-146. The spacer sequence can be fully complementary or substantially complementary (e.g., at least about 70% complementary (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more (e.g., 99.1, 99.2, 99.3, 99.4, 99.5, 99.6, 99.7, 99.8, 99.9, or 100%)) to a target nucleic acid. Thus, in some embodiments, the spacer sequence can have one, two, three, four, or five mismatches as compared to the target nucleic acid, which mismatches can be contiguous or noncontiguous. In some embodiments, the spacer sequence can have 70% complementarity to a target nucleic acid. In other embodiments, the spacer nucleotide sequence can have 80% complementarity to a target nucleic acid. In still other embodiments, the spacer nucleotide sequence can have 85%, 90%, 95%, 96%, 97%, 98%, 99% or 99.5% complementarity, and the like, to the target nucleic acid (protospacer). In some embodiments, the spacer sequence is 100% complementary to the target nucleic acid. A spacer sequence may have a length from about 15 nucleotides to about 30 nucleotides (e.g., 15, 16, 17, 18, 19, 20, 21, 22, 23, 24, 25, 26, 27, 28, 29, or 30 nucleotides, or any range or value therein). Thus, in some embodiments, a spacer sequence may have complete complementarity or substantial complementarity over a region of a target nucleic acid (e.g., protospacer) that is at least about 15 nucleotides to about 30 nucleotides in length. In some embodiments, the spacer is about 20 nucleotides in length. In some embodiments, the spacer is about 21, 22, or 23 nucleotides in length.

In some embodiments, the 5′ region of a spacer sequence of a guide nucleic acid may be identical to a target DNA, while the 3′ region of the spacer may be substantially complementary to the target DNA (e.g., Type V CRISPR-Cas), or the 3′ region of a spacer sequence of a guide nucleic acid may be identical to a target DNA, while the 5′ region of the spacer may be substantially complementary to the target DNA (e.g., Type II CRISPR-Cas), and therefore, the overall complementarity of the spacer sequence to the target DNA may be less than 100%. Thus, for example, in a guide for a Type V CRISPR-Cas system, the first 1, 2, 3, 4, 5, 6, 7, 8, 9, 10 nucleotides in the 5′ region (i.e., seed region) of, for example, a 20 nucleotide spacer sequence may be 100% complementary to the target DNA, while the remaining nucleotides in the 3′ region of the spacer sequence are substantially complementary (e.g., at least about 70% complementary) to the target DNA. In some embodiments, the first 1 to 8 nucleotides (e.g., the first 1, 2, 3, 4, 5, 6, 7, 8, nucleotides, and any range therein) of the 5′ end of the spacer sequence may be 100% complementary to the target DNA, while the remaining nucleotides in the 3′ region of the spacer sequence are substantially complementary (e.g., at least about 50% complementary (e.g., 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to the target DNA.

As a further example, in a guide for a Type II CRISPR-Cas system, the first 1, 2, 3, 4, 5, 6, 7, 8, 9, 10 nucleotides in the 3′ region (i.e., seed region) of, for example, a 20 nucleotide spacer sequence may be 100% complementary to the target DNA, while the remaining nucleotides in the 5′ region of the spacer sequence are substantially complementary (e.g., at least about 70% complementary) to the target DNA. In some embodiments, the first 1 to 10 nucleotides (e.g., the first 1, 2, 3, 4, 5, 6, 7, 8, 9, 10 nucleotides, and any range therein) of the 3′ end of the spacer sequence may be 100% complementary to the target DNA, while the remaining nucleotides in the 5′ region of the spacer sequence are substantially complementary (e.g., at least about 50% complementary (e.g., at least about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more or any range or value therein)) to the target DNA.

In some embodiments, a seed region of a spacer may be about 8 to about 10 nucleotides in length, about 5 to about 6 nucleotides in length, or about 6 nucleotides in length.

As used herein, a “target nucleic acid”, “target DNA,” “target nucleotide sequence,” “target region,” or a “target region in the genome” refers to a region of a plant’s genome that is fully complementary (100% complementary) or substantially complementary (e.g., at least 70% complementary (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a spacer sequence in a guide nucleic acid of this invention. A target region useful for a CRISPR-Cas system may be located immediately 3′ (e.g., Type V CRISPR-Cas system) or immediately 5′ (e.g., Type II CRISPR-Cas system) to a PAM sequence in the genome of the organism (e.g., a plant genome). A target region may be selected from any region of at least 15 consecutive nucleotides (e.g., 16, 17, 18, 19, 20, 21, 22, 23, 24, 25, 26, 27, 28, 29, 30 nucleotides, and the like) located immediately adjacent to a PAM sequence.

A “protospacer sequence” refers to the target double stranded DNA and specifically to the portion of the target DNA (e.g., or target region in the genome) that is fully or substantially complementary (and hybridizes) to the spacer sequence of the CRISPR repeat-spacer sequences (e.g., guide nucleic acids, CRISPR arrays, crRNAs).

In the case of Type V CRISPR-Cas (e.g., Cas12a) systems and Type II CRISPR-Cas (Cas9) systems, the protospacer sequence is flanked by (e.g., immediately adjacent to) a protospacer adjacent motif (PAM). For Type IV CRISPR-Cas systems, the PAM is located at the 5′ end on the non-target strand and at the 3′ end of the target strand (see below, as an example).

5'-NNNNNNNNNNNNNNNNNNN-3' RNA Spacer (SEQ ID NO:42
) | | | | | | | |||||| | || || |

3'AAANNNNNNNNNNNNNNNNNNN-5' Target strand (SEQ ID
NO:43) | | | |

5'TTTNNNNNNNNNNNNNNNNNNN-3' Non-target strand (SEQ
ID NO:44)

In the case of Type II CRISPR-Cas (e.g., Cas9) systems, the PAM is located immediately 3′ of the target region. The PAM for Type I CRISPR-Cas systems is located 5′ of the target strand. There is no known PAM for Type III CRISPR-Cas systems. Makarova et al. describes the nomenclature for all the classes, types and subtypes of CRISPR systems (Nature Reviews Microbiology 13:722-736 (2015)). Guide structures and PAMs are described in by R. Barrangou (Genome Biol. 16:247 (2015)).

Canonical Cas12aPAMs are T rich. In some embodiments, a canonical Cas12aPAM sequence may be 5′-TTN, 5′-TTTN, or 5′-TTTV. In some embodiments, canonical Cas9 (e.g., S. pyogenes) PAMs may be 5′-NGG-3′. In some embodiments, non-canonical PAMs may be used but may be less efficient.

Additional PAM sequences may be determined by those skilled in the art through established experimental and computational approaches. Thus, for example, experimental approaches include targeting a sequence flanked by all possible nucleotide sequences and identifying sequence members that do not undergo targeting, such as through the transformation of target plasmid DNA (Esvelt et al. 2013. Nat. Methods 10:1116-1121; Jiang et al. 2013. Nat. Biotechnol. 31:233-239). In some aspects, a computational approach can include performing BLAST searches of natural spacers to identify the original target DNA sequences in bacteriophages or plasmids and aligning these sequences to determine conserved sequences adjacent to the target sequence (Briner and Barrangou. 2014. Appl. Environ. Microbiol. 80:994-1001; Mojica et al. 2009. Microbiology 155:733-740).

In some embodiments, the present invention provides expression cassettes and/or vectors comprising the nucleic acid constructs of the invention (e.g., one or more components of an editing system of the invention). In some embodiments, expression cassettes and/or vectors comprising the nucleic acid constructs of the invention and/or one or more guide nucleic acids may be provided. In some embodiments, a nucleic acid construct of the invention encoding a base editor (e.g., a construct comprising a CRISPR-Cas effector protein and a deaminase domain (e.g., a fusion protein)) or the components for base editing (e.g., a CRISPR-Cas effector protein fused to a peptide tag or an affinity polypeptide, a deaminase domain fused to a peptide tag or an affinity polypeptide, and/or a UGI fused to a peptide tag or an affinity polypeptide), may be comprised on the same or on a separate expression cassette or vector from that comprising the one or more guide nucleic acids. When the nucleic acid construct encoding a base editor or the components for base editing is/are comprised on separate expression cassette(s) or vector(s) from that comprising the guide nucleic acid, a target nucleic acid may be contacted with (e.g., provided with) the expression cassette(s) or vector(s) encoding the base editor or components for base editing in any order from one another and the guide nucleic acid, e.g., prior to, concurrently with, or after the expression cassette comprising the guide nucleic acid is provided (e.g., contacted with the target nucleic acid).

Fusion proteins of the invention may comprise sequence-specific nucleic acid binding domains (e.g., sequence-specific DNA binding domains), CRISPR-Cas polypeptides, and/or deaminase domains fused to peptide tags or affinity polypeptides that interact with the peptide tags, as known in the art, for use in recruiting the deaminase to the target nucleic acid. Methods of recruiting may also comprise guide nucleic acids linked to RNA recruiting motifs and deaminases fused to affinity polypeptides capable of interacting with RNA recruiting motifs, thereby recruiting the deaminase to the target nucleic acid. Alternatively, chemical interactions may be used to recruit polypeptides (e.g., deaminases) to a target nucleic acid.

A peptide tag (e.g., epitope) useful with this invention may include, but is not limited to, a GCN4 peptide tag (e.g., Sun-Tag), a c-Myc affinity tag, an HA affinity tag, a His affinity tag, an S affinity tag, a methionine-His affinity tag, an RGD-His affinity tag, a FLAG octapeptide, a strep tag or strep tag II, a V5 tag, and/or a VSV-G epitope. Any epitope that may be linked to a polypeptide and for which there is a corresponding affinity polypeptide that may be linked to another polypeptide may be used with this invention as a peptide tag. In some embodiments, a peptide tag may comprise 1 or 2 or more copies of a peptide tag (e.g., repeat unit, multimerized epitope (e.g., tandem repeats)) (e.g., 1, 2, 3, 4, 5, 6, 7, 8, 9, 10, 11, 12, 13, 14, 15, 16, 17, 18, 19, 20, 21, 22, 23, 24, 25 or more repeat units. In some embodiments, an affinity polypeptide that interacts with/binds to a peptide tag may be an antibody. In some embodiments, the antibody may be a scFv antibody. In some embodiments, an affinity polypeptide that binds to a peptide tag may be synthetic (e.g., evolved for affinity interaction) including, but not limited to, an affibody, an anticalin, a monobody and/or a DARPin (see, e.g., Sha et al., Protein Sci. 26(5):910-924 (2017)); Gilbreth (Curr Opin Struc Biol 22(4):413-420 (2013)), U.S. Pat. No. 9,982,053, each of which are incorporated by reference in their entireties for the teachings relevant to affibodies, anticalins, monobodies and/or DARPins. Example peptide tag sequences and their affinity polypeptides include, but are not limited to, the amino acid sequences of SEQ ID NOs:45-47.

In some embodiments, a guide nucleic acid may be linked to an RNA recruiting motif, and a polypeptide to be recruited (e.g., a deaminase) may be fused to an affinity polypeptide that binds to the RNA recruiting motif, wherein the guide binds to the target nucleic acid and the RNA recruiting motif binds to the affinity polypeptide, thereby recruiting the polypeptide to the guide and contacting the target nucleic acid with the polypeptide (e.g., deaminase). In some embodiments, two or more polypeptides may be recruited to a guide nucleic acid, thereby contacting the target nucleic acid with two or more polypeptides (e.g., deaminases). Example RNA recruiting motifs and their affinity polypeptides include, but are not limited to, the sequences of SEQ ID NOs:48-58.

In some embodiments, a polypeptide fused to an affinity polypeptide may be a reverse transcriptase and the guide nucleic acid may be an extended guide nucleic acid linked to an RNA recruiting motif. In some embodiments, an RNA recruiting motif may be located on the 3′ end of the extended portion of an extended guide nucleic acid (e.g., 5′-3′, repeat-spacer-extended portion (RT template-primer binding site)-RNA recruiting motif). In some embodiments, an RNA recruiting motif may be embedded in the extended portion.

In some embodiments of the invention, an extended guide RNA and/or guide RNA may be linked to one or to two or more RNA recruiting motifs (e.g., 1, 2, 3, 4, 5, 6, 7, 8, 9, 10 or more motifs; e.g., at least 10 to about 25 motifs), optionally wherein the two or more RNA recruiting motifs may be the same RNA recruiting motif or different RNA recruiting motifs. In some embodiments, an RNA recruiting motif and corresponding affinity polypeptide may include, but is not limited, to a telomerase Ku binding motif (e.g., Ku binding hairpin) and the corresponding affinity polypeptide Ku (e.g., Ku heterodimer), a telomerase Sm7 binding motif and the corresponding affinity polypeptide Sm7, an MS2 phage operator stem-loop and the corresponding affinity polypeptide MS2 Coat Protein (MCP), a PP7 phage operator stem-loop and the corresponding affinity polypeptide PP7 Coat Protein (PCP), an SfMu phage Com stem-loop and the corresponding affinity polypeptide Com RNA binding protein, a PUF binding site (PBS) and the affinity polypeptide Pumilio/fem-3 mRNA binding factor (PUF), and/or a synthetic RNA-aptamer and the aptamer ligand as the corresponding affinity polypeptide. In some embodiments, the RNA recruiting motif and corresponding affinity polypeptide may be an MS2 phage operator stem-loop and the affinity polypeptide MS2 Coat Protein (MCP). In some embodiments, the RNA recruiting motif and corresponding affinity polypeptide may be a PUF binding site (PBS) and the affinity polypeptide Pumilio/fem-3 mRNA binding factor (PUF).

In some embodiments, the components for recruiting polypeptides and nucleic acids may those that function through chemical interactions that may include, but are not limited to, rapamycin-inducible dimerization of FRB - FKBP; Biotin-streptavidin; SNAP tag; Halo tag; CLIP tag; DmrA-DmrC heterodimer induced by a compound; bifunctional ligand (e.g., fusion of two protein-binding chemicals together, e.g., dihyrofolate reductase (DHFR).

In some embodiments, the nucleic acid constructs, expression cassettes or vectors of the invention that are optimized for expression in a plant may be about 70% to 100% identical (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to the nucleic acid constructs, expression cassettes or vectors comprising the same polynucleotide(s) but which have not been codon optimized for expression in a plant.

Further provided are nucleic acids comprising mutated Ribosomal Protein L3 (RPL3) genes, optionally wherein the mutated RPL3 genes comprises a mutation in Exon 3.

A further aspect provides a mutated nucleic acid encoding a Ribosomal Protein L3 (RPL3) polypeptide, optionally the mutation is in Exon 3 of the RPL3 gene, wherein the mutation results in no detectable RPL3 polypeptide or a RPL3 polypeptide having reduced binding to a trichothecene mycotoxin.

Further provided herein are cells comprising one or more polynucleotides, guide nucleic acids, nucleic acid constructs, expression cassettes or vectors of the invention.

The nucleic acid constructs of the invention (e.g., a construct comprising a sequence specific DNA binding domain, a CRISPR-Cas effector domain, a deaminase domain, reverse transcriptase (RT), RT template and/or a guide nucleic acid, etc.) and expression cassettes/vectors comprising the same may be used as an editing system of this invention for modifying target nucleic acids and/or their expression.

A target nucleic acid of a plant or plant part (or groupings of plants, for example, into a genus or higher order classification) may be modified (e.g., mutated, e.g., base edited, cleaved, nicked, etc.) using the polypeptides, polynucleotides, ribonucleoproteins (RNPs), nucleic acid constructs, expression cassettes, and/or vectors of the invention including a monocot. In some embodiments, the monocot plant is a wheat plant, an oat plant, a spelt plant and/or a barley plant. A plant and/or plant part that may be modified as described herein may be a plant and/or plant part of any wheat, oat, spelt or barley species/variety/cultivar.

The term “plant part,” as used herein, includes but is not limited to reproductive tissues (e.g., petals, sepals, stamens, pistils, receptacles, anthers, pollen, flowers, fruits, flower bud, ovules, seeds, embryos, nuts, kernels, ears, cobs and husks); vegetative tissues (e.g., petioles, stems, roots, root hairs, root tips, pith, coleoptiles, stalks, shoots, branches, bark, apical meristem, axillary bud, cotyledon, hypocotyls, and leaves); vascular tissues (e.g., phloem and xylem); specialized cells such as epidermal cells, parenchyma cells, chollenchyma cells, schlerenchyma cells, stomates, guard cells, cuticle, mesophyll cells; callus tissue; and cuttings. The term “plant part” also includes plant cells, including plant cells that are intact in plants and/or parts of plants, plant protoplasts, plant tissues, plant organs, plant cell tissue cultures, plant calli, plant clumps, and the like. As used herein, “shoot” refers to the above ground parts including the leaves and stems. As used herein, the term “tissue culture” encompasses cultures of tissue, cells, protoplasts and callus.

As used herein, “plant cell” refers to a structural and physiological unit of the plant, which typically comprise a cell wall but also includes protoplasts. A plant cell of the present invention can be in the form of an isolated single cell or can be a cultured cell or can be a part of a higher-organized unit such as, for example, a plant tissue (including callus) or a plant organ. In some embodiments, a plant cell can be an algal cell. A “protoplast” is an isolated plant cell without a cell wall or with only parts of the cell wall. Thus, in some embodiments of the invention, a transgenic cell comprising a nucleic acid molecule and/or nucleotide sequence of the invention is a cell of any plant or plant part including, but not limited to, a root cell, a leaf cell, a tissue culture cell, a seed cell, a flower cell, a fruit cell, a pollen cell, and the like. In some aspects of the invention, the plant part can be a plant germplasm. In some aspects, a plant cell can be non-propagating plant cell that does not regenerate into a plant.

“Plant cell culture” means cultures of plant units such as, for example, protoplasts, cell culture cells, cells in plant tissues, pollen, pollen tubes, ovules, embryo sacs, zygotes and embryos at various stages of development.

As used herein, a “plant organ” is a distinct and visibly structured and differentiated part of a plant such as a root, stem, leaf, flower bud, or embryo.

“Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

In some embodiments of the invention, a transgenic tissue culture or transgenic plant cell culture is provided, wherein the transgenic tissue or cell culture comprises a nucleic acid molecule/nucleotide sequence of the invention. In some embodiments, transgenes may be eliminated from a plant developed from the transgenic tissue or cell by breeding of the transgenic plant with a non-transgenic plant and selecting among the progeny for the plants comprising the desired gene edit and not the transgenes used in producing the edit.

Any barley plant, wheat plant, spelt plant or oat plant comprising an endogenous RPL3 gene may be modified as described herein to provide a plant having (1) a modified endogenous RPL3 gene that produces a RPL3 polypeptide with reduced binding to a trichothecene mycotoxin, (2) increased tolerance to deoxynivalenol (DON), (3) increased resistance/tolerance to fusarium head blight (FHB) (e.g., increased resistance to the FHB causal agent, Fusarium graminearum, and/or decreased FHB symptoms), (4) reduced Fusarium graminearum load, and/or (5) producing seed that accumulates less trichothecene mycotoxin (e.g., DON) when infected with Fusarium graminearum as compared to a plant devoid of the at least one mutation (e.g., an isogenic plant (e.g., wild type unedited plant or a null segregant). In some embodiments, the plant may be a wheat plant, and the least one mutation in an endogenous RPL3 gene may be in the A genome, optionally in chromosome 4A or in chromosome 5A, in the B genome, in the D genome, or in any combination thereof, optionally in the A genome and the B genome, optionally wherein the endogenous RPL3 gene is a RPL3A gene and/or a RPL3B gene. In some embodiments the RPL3A gene may be a RPL3A-1 gene, a RPL3A-2 gene, and/or a RPL3A-3 gene and/or the RPL3B gene is a RPL3B-1 gene, a RPL3B-2 gene, and/or a RPL3B-3 gene. In some embodiments, the at least one mutation may be a non-natural mutation.

In addition to a mutation in an endogenous RPL3 gene as described herein, a plant or part there of the invention may further comprise one or more additional Fusarium head blight resistance alleles. In some embodiments, the one or more additional Fusarium head blight resistance alleles may be one or more quantitative trait loci (QTL). At least 556 QTL are believed to be distributed on all sub-genomes and chromosomes of wheat. Thus, in some embodiments, a plant of this invention comprising a mutation in an RPL3 gene as described herein may further comprise one or more QTLs associated with Fusarium head blight resistance (e.g., stacked), wherein the QTL can include, but is not limited to, Fhb1, Fhb2, Fhb3, Fhb4, Fhb5, Fhb6, and/or Fhb7.

The invention will now be described with reference to the following examples. It should be appreciated that these examples are not intended to limit the scope of the claims to the invention but are rather intended to be exemplary of certain embodiments. Any variations in the exemplified methods that occur to the skilled artisan are intended to fall within the scope of the invention.

### EXAMPLES

### Example 1

A strategy was developed to generate truncations, knock-out or knock-down edits in the wheat Ribosomal Protein L3 genes of TraesCS4A02G208000 (SEQ ID NO:72), TraesCS4B02G111500 (SEQ ID NO:75), TraesCS4D02G109200 (SEQ ID NO:78), TraesCS5A02G112400 (SEQ ID NO:80), TraesCS5B02G118400 (SEQ ID NO:83), and/or TraesCS5D02G129200 (SEQ ID NO:86) to produce plants or parts thereof having increased resistance to infection by Fusarium head blight (FHB) (causal agent, Fusarium graminearum), increased resistance/tolerance to the fusarium mycotoxin deoxynivalenol (DON), increased resistance/tolerance to fusarium head blight (FHB) (e.g., increased resistance to the FHB causal agent, Fusarium graminearum, and/or decreased FHB symptoms), reduced Fusarium graminearum load, and/or to provide plants that produce seed or other plant parts, which accumulate less trichothecene mycotoxin. To generate a range of alleles, multiple Cas12a guide nucleic acids comprising spacers (SEQ ID NOs:140-146) (see Table 1) having complementarity to targets within one or more of the RPL3 genes (see Table 2) were designed and placed into a construct.

Lines carrying edits in the RPL3 genes were screened and those that showed about 10% of the sequencing reads having edits in the targeted gene were advanced to the next generation.

TABLE 1

Spacers
PWsp500 TACTCTCAACTCAGTCTGGGCAC SEQ ID NO:140
PWsp501 TACTCTCAACTCTGTCTGGGCAC SEQ ID NO:141
PWsp502 CACTCTCAACTCTGTCTGGGCTC SEQ ID NO:142
PWsp503 TCTTCTCAAGCTGCATATGGATT SEQ ID NO:143
PWsp504 AGTTATCGCCCATACCCAGGTCA SEQ ID NO:144
PWsp505 TTCTTGCCCGCATCACAGTCATA SEQ ID NO:145
PWsp506 TTCTTTCCTGAATCACTGTCATA - SEQ ID NO:146

TABLE 2

Alleles, genomic, cDNA, protein sequences and exemplary target regions for editing

| Endogenous gene/allele | Genomic Sequence SEQ ID NOs | cDNA Sequence SEQ ID NOs | Protein Sequence SEQ ID NOs | Genomic Target Regions SEQ ID NOs | cDNA Sequence Target Regions SEQ ID NOs |
|---|---|---|---|---|---|
| TraesCS4A02G208000 L3B-2 | 72 | 73 | 74 | 89-97 | 90-93, 96-100 |
| TraesCS4B02G111500 L3B-1 | 75 | 76 | 77 | 101-109 | - |
| TraesCS4D02G109200 L3B-3 | 78 | 79 | 77 | 110-113 | - |
| TraesCS5A02G112400 L3A-3 | 80 | 81 | 82 | 114-122 | 123-124 |
| TraesCS5B02G118400 L3A-2 | 83 | 84 | 85 | 125-133 | 134-135 |
| TraesCS5D02G129200 L3A-1 | 86 | 87 | 88 | 136-139 | - |

### Example 2. Edited Alleles of RPL3

The RPL3 genes in the edited plants generated as described in Example 1 were sequence analyzed and a range of alleles of RPL3 were identified and are summarized in Table 3.

TABLE 3

Edited alleles of RPL3

| | | |
|---|---|---|
| genotype | Edit description | Protein description |
| TraesCS4D02G109200 (Allele 1) (SEQ ID NO:147) | 2 bp deletion starting at position 1385 of SEQ ID NO:78 | Out of frame mutation leading to an early stop codon (SEQ ID NO:148) |
| TraesCS5A02G112400 (Allele 2) (SEQ ID NO:149) | 7 bp deletion starting at position 1211 of SEQ ID NO:80 | Out of frame mutation leading to an early stop codon (SEQ ID NO:150) |
| TraesCS4D02G109200 (Allele 3) (SEQ ID NO:151) | 7 bp deletion starting at position 1391 of SEQ ID NO:78 | Out of frame mutation leading to an early stop codon (SEQ ID NO:152) |
| TraesCS5D02G129200 (Allele 4) (SEQ ID NO:153) | 8 bp deletion starting at position 1371 of SEQ ID NO:86 | Out of frame mutation leading to an early stop codon (SEQ ID NO:154) |
| TraesCS4D02G109200 (Allele 1) (SEQ ID NO:155) | 1 bp insertion at position 1397 of SEQ ID NO:78 | Out of frame mutation leading to an early stop codon (SEQ ID NO:156) |

### Example 3. Evaluation Fusarium Disease in a Growth Chamber

Two gram of wheat seeds of each wheat cultivar/variety/line are sown in one-liter pots containing pasteurized soil medium. The wheat seeds are allowed to germinate in greenhouse conditions under fluorescent lighting with a 14-hour photoperiod. At the onset of wheat flowering, the wheat heads or a wheat spikelet are challenged by spraying with F. graminearum conidial spores, such as F. graminearum NRRL 5883. Conidial spores are harvested from five-day old PDA plates by pouring water with 0.01% Tween20 on plates and scraping spores into suspension. After spraying spores, wheat plants are transferred to a mist chamber with 100% humidity for three days to allow for infection.

Twenty days after infection, watering is stopped and wheat plants are allowed to dry for three weeks before harvesting. Individual wheat heads are harvested and collected. Disease severity is determined for each head showing head blight disease symptoms. Disease severity is calculated as the number of diseased spikelets divided by the total number of spikelets per heads.

The seeds from each head are harvested by hand and the seed mass is weighed. The average seed weight per pot in each of the treatments is calculated.

### Example 4. Evaluation of Fusarium Disease on Wheat Seedlings

Seeds of any wheat cultivar/variety/line are sown in 1-Liter pots each containing pasteurized soil medium in 20 cm diameter plastic pots. The seeds and plants were maintained in a greenhouse at room temperature with a 14 h photoperiod of light.

Mycelium of a Fusarium graminearum strain, such as NRRL-5883, is grown on PDA plates for 5 days under constant light. Hyphae and conidia are harvested by pouring a few mL of sterile water (0.01% Tween 20) on the plates and scraping the agar surface with a sterile spatula. The concentration of spores in the inoculum is adjusted to approximately 2×10⁵ spores/mL

Inoculation with F. graminearum begins after shoots emerge from the seeds and continues every other evening for 10 days. The inoculum of F. graminearum is applied with a sprayer at about 30 mL per pot. Immediately after each inoculation, the plants are misted with overhead mister.

Head blight is evaluated by the severity of Fusarium infestation, as determined by the number of Fusarium colony-forming units per gram (CFU/g) of plant tissues.

### Example 5. Evaluation of Fusarium Disease in a Greenhouse

Two gram of wheat seeds of each wheat cultivar/variety/line are sown in one-liter pots containing pasteurized soil medium. The wheat seeds are allowed to germinate in greenhouse conditions under fluorescent lighting with a 14-hour photoperiod. At flowering stage (Feekes 10.5.1) the wheat heads or a wheat spikelet are infected by spraying with a Fusarium graminearum conidial spore suspension. A fungal spore suspension can be prepared by plating homogenized mycelia of F. graminearum, such as strain NRRL 5883, onto PDA plates, followed by five-day incubation under constant light at room temperature. The fungal spores are then harvested by flooding the plates with magnesium sulfate buffer (10 mM MgSO₄; 0.01% Tween 20) and gently scraping the agar surface with a sterile spatula. The spore concentration is adjusted and approximately 50,000 conidial spores are applied per head with a pressurized hand sprayer. The growth room relative humidity is increased to 90% for three days allowing for infection before again being normalized. After wheat seeds have set, watering is stopped, and the wheat heads are allowed to dry completely. Individual wheat heads are harvested and collected. Disease severity is determined for each wheat head showing head blight disease symptoms. Disease severity is calculated as the number of diseased spikelets divided by the total number of spikelets per head.

### Example 6. Field Evaluation of Fusarium Disease

Field assessments of head blight disease and incidence are made by evaluating 60 heads per replicate (i e. 300 heads/treatment) when plant development reaches between mid-milk and soft dough development. Wheat heads are harvested by hand and threshed using an Almaco single plant and head thresher (Almaco, Iowa) when grains reach full maturity. Grain samples obtained from each replicate row are evaluated for 100-kernel weight. Disease severity, incidence, and 100-kernel weight data are analyzed using one-way analysis of variance (ANOVA).

The foregoing is illustrative of the present invention and is not to be construed as limiting thereof. The invention is defined by the following claims, with equivalents of the claims to be included therein.

What is claimed is:

1. A plant or part thereof comprising at least one (e.g., one or more) mutation in an endogenous Ribosomal Protein L3 (RPL3) gene encoding a RPL3 polypeptide, optionally, wherein the at least one mutation results in no detectable RPL3 polypeptide or a RPL3 polypeptide having reduced binding to a trichothecene mycotoxin.

2. The plant or part thereof of claim 1, wherein the trichothecene mycotoxin is deoxynivalenol (DON).

3-4. (canceled)

5. The plant or part thereof of claim 1, wherein the at least one mutation is a base substitution, a base deletion, and/or a base insertion, optionally, a base deletion or base insertion of at least one base pair, optionally a deletion or insertion of about 1 base pair to about 100 consecutive base pairs.

6-8. (canceled)

9. The plant or part thereof of claim 5, wherein the base deletion is an out-of-frame deletion and/or the base insertion is an out-of-frame insertion, optionally wherein the out-of-frame deletion and/or out-of-frame insertion results in a premature stop codon, optionally resulting in a truncated RPL3 polypeptide.

10-16. (canceled)

17. The plant or part thereof of claim 1, wherein the endogenous RPL3 gene is a RPL3A gene and/or a RPL3B gene, optionally the endogenous RPL3 gene is a RPL3A-1 gene, a RPL3A-2 gene, a RPL3A-3 gene, a RPL3B-1 gene, a RPL3B-2 gene, and/or a RPL3B-3 gene.

18-19. (canceled)

20. The plant or part thereof of claim 1, wherein the endogenous RPL3 gene (a) comprises a nucleotide sequence having at least 80% sequence identity to any one of the nucleotide sequences of SEQ ID NOs:72, 73, 75, 76, 78, 79, 80, 81, 83, 84, 86, or 87, (b) comprises a region having at least 80% sequence identity to any one of the nucleotide sequences of SEQ ID NOs:89-139; and/or (c) encodes a polypeptide having at least 80% sequence identity to any one of the amino acid sequences of SEQ ID NOs:74, 77, 82, 85, or 88.

21-22. (canceled)

23. The plant or part thereof of claim 1, wherein the plant or part thereof exhibits increased resistance to Fusarium head blight (FHB), exhibits a decreased amount of Fusarium graminearum, and/or exhibits an increased tolerance to deoxynivalenol (DON).

24-25. (canceled)

26. The plant or part thereof of claim 1, wherein the plant produces seed that accumulates less trichothecene mycotoxin (e.g., DON) upon infection with FHB (infection by the causal agent of FHB (Fusarium graminearum).

27-31. (canceled)

32. The plant or part thereof of claim 1, wherein the at least one mutation results in a mutated RPL3 gene having at least 90% sequence identity to any one of the nucleotide sequences of SEQ ID NOs:147, 149, 151, 153, or 155 and/or encoding a mutated RPL3 polypeptide having at least 90% sequence identity to any one of the amino acid sequences of SEQ ID NOs:148, 150, 152, 154, or 156.

33-41. (canceled)

42. A plant cell comprising at least one mutation in one or more endogenous Ribosomal Protein L3 (RPL3) genes, wherein the at least one mutation is a substitution, insertion and/or a deletion that is introduced using an editing system that comprises a nucleic acid binding domain that binds to a target site in the one or more endogenous RPL3 genes, wherein the endogenous RPL3 gene (a) comprises a nucleotide sequence having at least 80% sequence identity to any one of the nucleotide sequences of SEQ ID NOs:72, 73, 75, 76, 78, 79, 80, 81, 83, 84, 86, or 87, (b) comprises a region having at least 80% sequence identity to any one of the nucleotide sequences of SEQ ID NOs:89-139; and/or encodes a polypeptide having at least 80% sequence identity to any one of the amino acid sequences of SEQ ID NOs:74, 77, 82, 85, or 88 and the target site is within a region of the RPL3 gene comprising a sequence having at least 80% sequence identity to any one of the nucleotide sequences of SEQ ID NO:89-139.

43-44. (canceled)

45. The plant cell of claim 42, wherein the at least one mutation results in a knock-down or a knock-out of the endogenous RPL3 gene.

46-49. (canceled)

50. The plant cell of claim 42, wherein the at least one mutation within the one or more endogenous RPL3 genes is an insertion and/or a deletion, optionally the at least one mutation is an out-of-frame insertion or an out-of-frame deletion.

51. The plant cell of claim 42 wherein the at least one mutation is an out-of-frame insertion or an out-of-frame deletion that results in a premature stop codon, optionally a truncated protein.

52. (canceled)

53. The plant cell of claim 42, wherein the mutation results in a mutated RPL3 gene having at least 90% sequence identity to any one the nucleotide sequences of SEQ ID NOs:147, 149, 151, 153, or 155 and/or encoding a mutated RPL3 polypeptide having at least 90% sequence identity to any one of the amino acid sequences of SEQ ID NOs:148, 150, 152, 154, or 156.

54-74. (canceled)

75. A method for producing a plant or part thereof comprising at least one cell having a mutated endogenous Ribosomal Protein L3 (RPL3) gene, the method comprising contacting a target site in an endogenous RPL3 gene in the plant or plant part with a nuclease comprising a cleavage domain and a nucleic acid binding domain, wherein the nucleic acid binding domain binds to a target site in the endogenous RPL3 gene, wherein the endogenous RPL3 gene:
(a) comprises a sequence having at least 80% sequence identity to any one of the nucleotide sequences of SEQ ID NOs:72, 73, 75, 76, 78, 79, 80, 81, 83, 84, 86, or 87;
(b) comprises a region having at least 80% identity to any one of the nucleotide sequences of SEQ ID NOs:89-139; and/or
(c) encodes an amino acid sequence having at least 80% sequence identity to any one of the amino acid sequences of SEQ ID NOs:74, 77, 82, 85, or 88, thereby producing the plant or part thereof comprising at least one cell having a mutation in the endogenous RPL3 gene, optionally wherein the mutation in the endogenous RPL3 gene produces a RPL3 polypeptide having reduced binding to a trichothecene mycotoxin.

76-80. (canceled)

81. The method of claim 75, wherein the mutation is a knock-out or knock down mutation.

82. (canceled)

83. The method of claim 75, wherein the mutation is an out-of-frame insertion or an out-of-frame deletion, optionally wherein the mutation results in a premature stop codon, optionally resulting in a truncated protein.

84-88. (canceled)

89. The method of claim 75, wherein the plant having the mutation in the endogenous RPL3 gene exhibits increased resistance to fusarium head blight (FHB), a reduced Fusarium graminearum load, increased tolerance to deoxynivalenol (DON) and/or produces seed that accumulates less trichothecene mycotoxin when infected with Fusarium graminearum when compared to a control plant that is devoid of the mutation in the endogenous RPL3 gene.

90. (canceled)

91. The method of claim 75, wherein the at least one mutation results in a mutated RPL3 gene having at least 90% sequence identity to any one of the nucleotide sequences of SEQ ID NOs:147, 149, 151, 153, or 155 and/or encoding a mutated RPL3 polypeptide having at least 90% sequence identity to any one of the amino acid sequences of SEQ ID NOs:148, 150, 152, 154, or 156.

92-93. (canceled)

94. A guide nucleic acid that binds to a target site in a RPL3 gene, wherein the target site is in a region of the RPL3 gene having at least 80% sequence identity to any one of the nucleotide sequences of SEQ ID NOs:89-139, optionally wherein the guide nucleic acid comprises a spacer having the nucleotide sequence of any one of SEQ ID NOs:140-146.

95-115. (canceled)
